(19) United States
(12) Patent Application Publication
(10) Pub. No.: US 2024/0010624 A1
(43) Pub. Date: Jan. 11, 2024

(54) TRICYCLIC COMPOUNDS AND RELATED COMPOSITIONS, ZINC ELECTROCHEMICAL CELLS, BATTERIES, METHODS AND SYSTEMS

(71) Applicant: ALIONYX ENERGY SYSTEMS, Monrovia, CA (US)

(72) Inventors: Jasim UDDIN, Pasadena, CA (US); Simon C. JONES, Whittier, CA (US); Andrew STEWART, Pasadena, CA (US); Zeiad MUNTASSER, Glendale, CA (US)

(21) Appl. No.: 18/253,092

(22) PCT Filed: Nov. 19, 2021
(86) PCT No.: PCT/US2021/060184

§ 371 Date: May 16, 2023

Related U.S. Application Data
(60) Provisional application No. 63/215,827, filed on Jun. 28, 2021.
(60) Provisional application No. 63/116,123, filed on Nov. 19, 2020.

(51) Int. Cl. C07D 279/20; C08G 61/12; C07D 279/22; C08F 134/04; H01M 4/60; H01M 10/36

(52) U.S. Cl. CPC C07D 279/20; C08G 61/122; C07D 279/22; C08F 134/04; H01M 4/608; H01M 10/365; C08G 2261/11; C08G 2261/1412; C08G 2261/226; C08G 2261/228; C08G 2261/3246

(57) ABSTRACT

Redox active polycyclic compounds and related electrode material, electrode chemical cell battery, methods and systems are described. In particular, tricyclic compounds having a redox potential of 0.20 V to 2.0 V with reference to Zn/Zn2+ electrode potential under standard conditions are described. More particularly, redox active monomers, dimers, and polymers in which each monomeric unit contains a tricyclic heterocyclic structure are provided as electrode material of a cathode for an electrochemical cell further containing a zinc anode and an aqueous electrolyte.

### CROSS REFERENCE TO RELATED APPLICATIONS

The present application claims priority to U.S. Provisional Application No. 63/116,123, entitled “Organic Electrode Materials for Zinc Batteries and Their Applications” filed on Nov. 19, 2020 with docket number P2551-USP, and to U.S. Provisional Application No. 63/215,827, entitled “Organic Electrode Materials for Zinc Batteries and Their Applications” filed on Jun. 28, 2021 with docket number P2551-USP2, the content of each of which is incorporated herein by reference in its entirety.

### FIELD

The present disclosure relates to electrode active materials, and battery systems that feature electrodes incorporating organic materials. In particular, the present disclosure relates to organic molecules, polymers, crosslinked polymers and related compositions, electrochemical cells, batteries, methods and systems that can be used to improve electrochemical cells and batteries performance.

### BACKGROUND

Performance, economics and safety has been at the center of various efforts to improve electrode active materials and battery systems.

Despite progresses made in the recent years, however, production for high reliability, high capacity, long-life, cheap and/or safe energy storage devices is still challenging in particular reference to batteries in large-scale applications, for example in utility grid storage supporting renewable power generation or in full-home backup battery installations.

### SUMMARY

Described herein are polycyclic compounds, and related compositions, methods systems, as well as electrode material, electrodes, high capacity Zn electrochemical cells and batteries which, in several embodiments, allow production of high performance redox active materials which can be used as cathode active materials in high capacity, safe and long-lasting electrochemical cells and batteries with aqueous electrolytes.

According to a first aspect, a tricyclic compound is described, the tricyclic compound being represented by Formula (I)

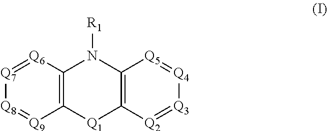

in which
Q1 is a —O—, —S— or ═NR4,
Q2 to Q9 are each independently selected from N or CR5 with the proviso that at most two of Q2 to Q5 or two of Q6 to Q9 are N,
wherein R1, and R4 are independently selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein the tricyclic compound as described has a redox potential of 0.20 V to 2.0 V with reference to Zn/Zn²⁺ electrode potential under standard conditions.

According to a second aspect, a tricyclic compound comprising two three-ring structures is described, the tricyclic compound being represented by Formula (IV)

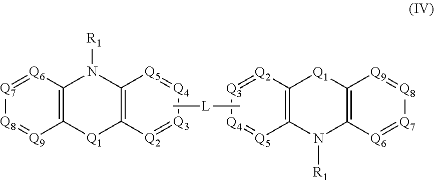

in which
Q1 is a —O—, —S— or ═NR4,
Q2 to Q5 are each independently selected from N or CR5 with the proviso that at most two of Q2 to Q5 are N and one of Q2 to Q5 is C—X wherein X is Cl, Br, or I,
Q6 to Q9 are each independently selected from N or CR5 with the proviso that at most two of Q6 to Q9 are N,
wherein R1, and R4 are independently selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S;
wherein L is null when a coupling reagent including CuI is used or O, S, NR1, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S, wherein L links between a ring carbon atom of any one of Q2 to Q5 to a ring carbon atom of any one of Q2 to Q5 of another monomeric moiety,
wherein the tricyclic compound as described has a redox potential of 0.20 V to 2.0 V with reference to Zn/Zn²⁺ electrode potential under standard conditions.

According to a third aspect, a tricyclic compound comprising three or more three-ring structures is described, the tricyclic compound being represented by Formula (II)

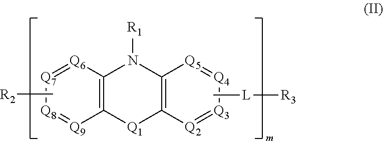

in which
Q1 is a —O—, —S— or ═NR4,
Q2 to Q9 are each independently selected from N or CR5 with the proviso that at most two of Q2 to Q5 or two of Q6 to Q9 are N,
wherein R1, and R4 are independently selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group. C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
L is null or O, S, NR1, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S, wherein L links between a ring carbon atom of any one of Q2 to Q5 to a ring carbon atom of any one of Q6 to Q9 of an adjacent monomeric moiety,
R2 and R3 are null or H,
m ranges from 3 to 10,000,
wherein the tricyclic compound and related polymer are described have a redox potential of 0.20 V to 2.0 V with reference to Zn/Zn²⁺ electrode potential under standard conditions.

According to a fourth aspect, a tricyclic compound comprising three or more three-ring structure is described, the tricyclic compound being represented by Formula (VII)

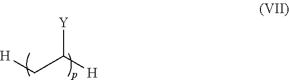

wherein Y is selected from any one of Formula (10a), Formula (10b) and Formula (10c)

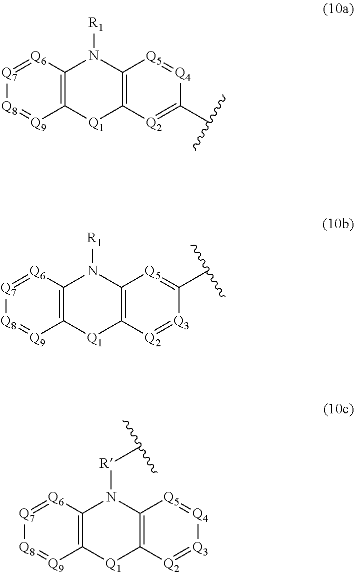

wherein
Q1 is a —O—, —S— or ═NR4,
Q2 to Q5 are each independently selected from N or CR5 with the proviso that at most two of Q2 to Q5 are N and one of Q2 to Q5 is C—X wherein X is Cl, Br, or I,
Q6 to Q9 are each independently selected from N or CR5 with the proviso that at most two of Q6 to Q9 are N,
wherein R′ is selected from a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R1, and R4 are independently selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein p ranges from 3 to 10,000, wherein the tricyclic compound as described has a redox potential of 0.20 V to 2.0 V with reference to Zn/Zn²⁺ electrode potential under standard conditions.

According to a fifth aspect a method is described for making a tricyclic compound comprising two three-ring structures, the method comprising
providing a tricyclic monomer of Formula (III)

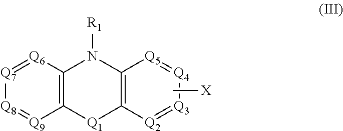

in which
Q1 is a —O—, —S— or ═NR4,
Q2 to Q5 are each independently selected from N or CR5 with the proviso that at most two of Q2 to Q5 are N and one of Q2 to Q5 is C—X wherein X is Cl, Br, or I,
Q6 to Q9 are each independently selected from N or CR5 with the proviso that at most two of Q6 to Q9 are N,
wherein R1, and R4 are independently selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
contacting the tricyclic monomer of Formula (III) with CuI or other coupling reagent capable of performing a carbon-carbon bond formation reaction for a time and under conditions to allow reaction of the tricyclic monomer of Formula (III) with CuI or the other coupling reagent, to provide the dimer of Formula (IV) herein described,
   or
contacting the tricyclic monomer of Formula (III) with a salt for linker L such as Na2S, K2S, Li2S for a time and under conditions to allow reaction of the tricyclic monomer of Formula (III) with the salt for linker L such as Na2S, K2S, Li2S, to provide the dimer of Formula (IV) herein described,
   wherein L is null when CuI or other coupling reagent is used or O, S, NR1, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S, wherein L links between a ring carbon atom of any one of Q2 to Q5 to a ring carbon atom of any one of Q2 to Q5 of another monomeric moiety,

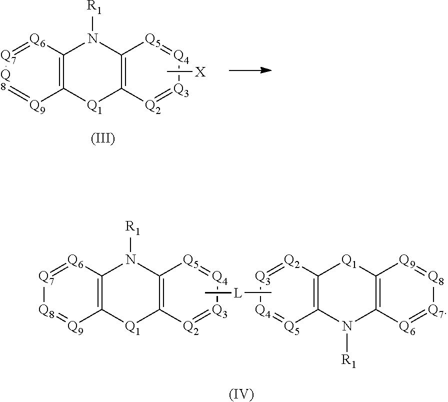

According to a sixth aspect a method is described for making a tricyclic compound comprising three or more three-ring structures, the method comprising
providing a tricyclic monomer of Formula (V)

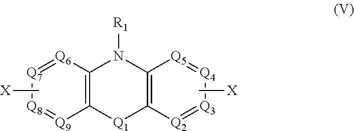

in which
Q1 is a —O—, —S— or ═NR4,
Q2 to Q9 are each independently selected from N or CR5 with the proviso that at most two of Q2 to Q5 or at most two of Q6 to Q9 are N and one of Q2 to Q5 and one of Q6 to Q9 are C—X wherein X is Cl, Br, or I.
wherein R1, and R4 are independently selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
contacting the tricyclic monomer of Formula (V) with a coupling reagent capable of performing a carbon-carbon bond formation reaction such as CuI for a time and under conditions to allow reaction of the tricyclic monomer of Formula (V) with the coupling reagent to provide the polymer of Formula (II) herein described, or
contacting the tricyclic monomer of Formula (V) with a suitable salt of linker L such as any one of Na2S, Li2S, K2S for a time and under conditions to allow reaction of the tricyclic monomer of Formula (V) with a salt for linker L to provide the polymer of Formula (II) herein described,
wherein L is null when the coupling reagent is used, or O, S, NR1, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S, wherein L links between a ring carbon atom of any one of Q2 to Q5 to a ring carbon atom of any one of Q6 to Q9 of an adjacent monomeric moiety,
wherein R2 and R3 are null or H, and
m ranges from 3 to 10,000,

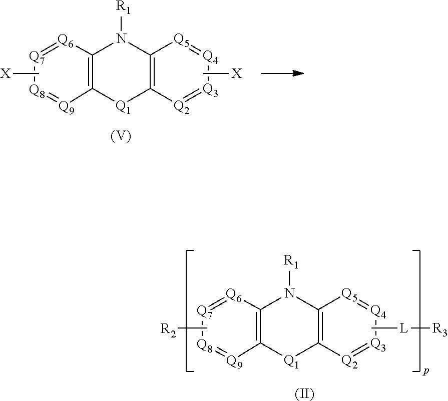

According to a seventh aspect a method is described for making a tricyclic compound comprising three or more three-ring structures of Formula (VII), the method comprising
providing a tricyclic monomer of Formula (VI)

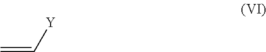

wherein Y is selected from any one of Formula (10a), Formula (10b) and Formula (10c)

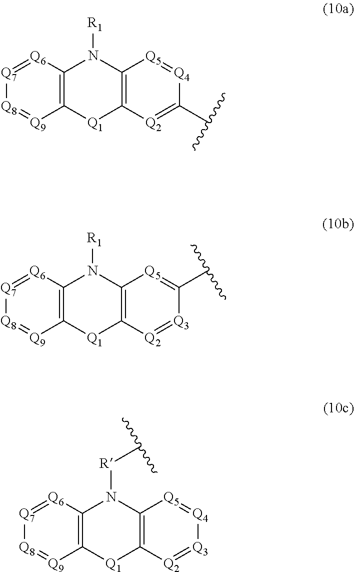

wherein
Q1 is a —O—, —S— or ═NR4,
Q2 to Q5 are each independently selected from N or CR5 with the proviso that at most two of Q2 to Q5 are N and one of Q2 to Q5 is C—X wherein X is Cl, Br, or I,
Q6 to Q9 are each independently selected from N or CR5 with the proviso that at most two of Q6 to Q9 are N,
wherein R′ is selected from a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R1, and R4 are independently selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
contacting the tricyclic monomer of Formula (VI) with a polymerization initiator or catalyst for a time and under conditions to allow polymerization of the tricyclic monomer of Formula (VI) to provide a polymer of Formula (VII) herein described,

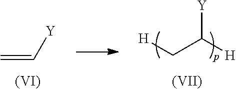

wherein p ranges from 3 to 10,000.

According to a eighth aspect an electrode composition is described, the electrode composition comprising a tricyclic compound of Formula (I), Formula (II), Formula (IV), or Formula (VII) herein described and/or a crosslinked polymer of Formula (II) and Formula (VII) herein described, together with a binder, and a conductive additive.

According to a nineth aspect, an electrochemical cell is described, the electrochemical cell comprising a zinc anode, a cathode, current collectors, external housing, a separator and an aqueous electrolyte, wherein the cathode electrode comprises the tricylic compound of Formula (I), Formula (II), Formula (IV), or Formula (VII) herein described and/or a crosslinked polymer of Formula (II) and Formula (VII) herein described.

According to a tenth aspect, a battery is described, the battery comprising at least one electrochemical cell herein described.

The monomer, dimer and polymers and related compositions electrochemical cells methods and systems, allow in several embodiments to provide batteries with a high capacity (at least 50 mAh/g for active material or redox active network polymer that is utilized), long life-time (e.g. at least 4 years) and/or low safety hazard including low flammability.

The tricyclic compounds herein described and related compositions electrochemical cells methods and systems allow in several embodiments to provide batteries with low spatial footprint and low replacement.

The tricyclic compounds herein described and related compositions electrochemical cells methods and systems as described herein allow in several embodiments to provide batteries having a higher capacity, longer life-time and/or reduced safety hazards with respect to existing lead-acid batteries.

In particular the tricyclic compounds herein described and related compositions electrochemical cells methods and systems, allow in several embodiments to provide Zn aqueous batteries having a comparable or higher capacity, longer life time and reduced safety hazards with particular reference to lead-acid batteries, and lithium-ion batteries using considerable quantities of flammable organic solvent electrolyte of at least 1 mL/Ah in large batteries (having 5 kWh or more, 25 kWh or more, 50 kWh or more).

Additionally, the tricyclic compounds herein described and related compositions electrochemical cells methods and systems, allow in several embodiments to provide Zn aqueous batteries having a comparable or higher capacity, and longer lifetime with respect to existing batteries based on organic redox materials.

The tricyclic compounds herein described and related compositions electrochemical cells methods and systems herein described can be used in connection with applications wherein electrochemical cell with high capacity, long life low safety hazards, low spatial footprint and/or low replacement are desired. Exemplary applications comprise batteries for grid storage, telecommunication, automotive start-stop.

The details of one or more embodiments of the disclosure are set forth in the accompanying drawings and the description below. Other features and objects will be apparent from the description and drawings, and from the claims.

### BRIEF DESCRIPTION OF DRAWINGS

The accompanying drawings, which are incorporated into and constitute a part of this specification, illustrate one or more embodiments of the present disclosure and, together with the description of example embodiments, serve to explain the principles and implementations of the disclosure.

FIG. 1 shows a schematic representation of a comparison the functioning of an electrochemical cells comprising electrodes of the present disclosure (PolyZ) compared to existing zinc batteries (Zn/Br₂) in aqueous electrolyte.

FIG. 2 shows a schematic representation of a working mechanism for a an electrochemical cell according to the present disclosure wherein the tricyclic compounds are not soluble in the aqueous electrolyte.

FIG. 3 shows a chart showing the electrochemical stability window of water in 3M Zn(OTf)₂, which was found to be ˜1.6V.

FIG. 4 shows that the cyclic voltammetry (CV) data of a tricycling organic molecule, phenothiazine (PT) at 10 mV/s. The data shows a remarkable stability of PT in 3M Zn(OTf)₂ electrolyte. The CV was cycled for over 600 times at 10 mV/s scan rate. A slightly higher current was observed at cycle 600 than at cycle 100.

FIG. 5 shows the voltage profile of a zinc/phenothiazine (PT) electrochemical cell using 3M Zn(OTf)₂ aqueous electrolyte during a charging and discharging cycle.

FIG. 6 shows change of discharge capacity of a zinc/phenothiazine (PT) electrochemical cell using 3M Zn(OTf)₂ aqueous electrolyte over a plurality of charging and discharging cycles.

FIG. 7 shows a change of coulombic efficiency of a zinc/phenothiazine (PT) electrochemical cell using 3M Zn(OTf)₂ aqueous electrolyte over a plurality of charging and discharging cycles.

FIG. 8 shows a cell voltage profile of a zinc/phenothiazine (PT) electrochemical cell in 2M Zn(OTf)₂+1M LiTFSI in H₂O electrolyte. Cell was cycled at 2 C rate.

FIG. 9 shows a change of discharge capacity of a zinc/phenothiazine (PT) electrochemical cell in 2M Zn(OTf)₂+1M LiTFSI in H₂O aqueous electrolyte over a plurality of charging and discharging cycles.

FIG. 10 shows a change of Coulombic efficiency of a zinc/phenothiazine (PT) electrochemical cell in 2M Zn(OTf)₂+1M LiTFSI in H₂O aqueous electrolyte over a plurality of charging and discharging cycles.

FIG. 11 the voltage profile of a zinc/phenothiazine (PT) electrochemical cell using 23 wt % of Zn(ClO₄)₂, 16 wt % of LiTFSI, 7 wt % of NaClO₄ in H₂O aqueous electrolyte during a charging and discharging cycle.

FIG. 12 shows a change of discharge capacity of a zinc/phenothiazine (PT) electrochemical cell using 23 wt % of Zn(ClO₄)₂, 16 wt % of LiTFSI, 7 wt % of NaClO₄ in H₂O aqueous electrolyte over a plurality of charging and discharging cycles.

FIG. 13 shows a change of coulombic efficiency of a zinc/phenothiazine (PT) electrochemical cell using 23 wt % of Zn(ClO₄)₂, 16 wt % of LiTFSI, 7 wt % of NaClO₄ in H₂O aqueous electrolyte over a plurality of charging and discharging cycles

FIG. 14 shows a reversible redox peak for PT₂S in cyclic voltammetry (CV) experiment in 23 wt % of Zn(ClO₄) 2, 16 wt % of LiTFSI, 7 wt % of NaClO₄ in H₂O aqueous electrolyte. The zinc plating and stripping was observed at 0.00V, and PT₂S redox process was observed at 1.25V vs. Zn/Zn²⁺.

FIG. 15 shows the voltage profile of zinc/PT₂S electrochemical cell using 23 wt % of Zn(ClO₄)₂, 16 wt % of LiTFSI, 7 wt % of NaClO₄ in H₂O aqueous electrolyte during a charging and discharging cycle.

FIG. 16 shows a change of discharge capacity of zinc/PT₂S electrochemical cell using 23 wt % of Zn(ClO₄)₂, 16 wt % of LiTFSI, 7 wt % of NaClO₄ in H₂O aqueous electrolyte over a plurality of charging and discharging cycles.

FIG. 17 shows a change of coulombic efficiency of zinc/PT₂S electrochemical cell using 23 wt % of Zn(ClO₄)₂. 16 wt % of LiTFSI, 7 wt % of NaClO₄ in H₂O aqueous electrolyte over a plurality of charging and discharging cycles.

FIG. 18 shows a reversible redox potential for PMPTS polymer in cyclic voltammetry (CV) experiment at 10 mV/s in 3M Zn(OTf)₂/H₂O electrolyte. The plating and stripping for metallic zinc were found to be at 0.00V vs Zn/Zn²⁺ as expected, and redox process for PMPTS polymer was found to be at 1.55V vs. Zn/Zn²⁺.,. The redox potential for PMPTS is about 300 mV higher than that of PT molecule or PT₂S molecule (FIGS. 4 and 14). The improved voltage is attributed in part to the presence of an N-methyl group in the molecule, and —S— linkage.

FIG. 19 shows the voltage profile of zinc/PMPTS polymer electrochemical cell using 3M Zn(OTf)₂ aqueous electrolyte during a charging and discharging cycle at 5 C. A 1.50V battery was obtained.

FIG. 20 shows a change of normalized discharged capacity of zinc/PMPTS polymer electrochemical cell using 3M Zn(OTf)₂ aqueous electrolyte during a charging and discharging cycle.

FIG. 21 shows a change of coulombic efficiency of zinc/PMPTS polymer electrochemical cell using 3M Zn(OTf)₂ aqueous electrolyte during a charging and discharging cycle.

FIG. 22 shows the voltage profile of a zinc/PMPTS electrochemical cell using 23 wt % of Zn(ClO₄)₂, 16 wt % of LiTFSI, 7 wt % of NaClO₄ in H₂O aqueous electrolyte during a charging and discharging cycle at 5 C rate.

FIG. 23 shows a change of discharge capacity of a zinc/PMPTS electrochemical cell using 23 wt % of Zn(ClO₄)₂, 16 wt % of LiTFSI, 7 wt % of NaClO₄ in H₂O aqueous electrolyte over a plurality of charging and discharging cycles at 5 C rate.

FIG. 24 shows a change of Coulombic efficiency of a zinc/PMPTS electrochemical cell using 23 wt % of Zn(ClO₄)₂, 16 wt % of LiTFSI, 7 wt % of NaClO₄ in H₂O aqueous electrolyte over a plurality of charging and discharging cycles at 5 C rate.

FIG. 25 shows the voltage profile of a zinc/PMPTS electrochemical cell using 23 wt % of Zn(ClO₄)₂, 16 wt % of LiTFSI, 7 wt % of NaClO₄ in H₂O aqueous electrolyte during a charging and discharging cycle at 2 C rate.

FIG. 26 shows a change of normalized discharge capacity of a zinc/PMPTS electrochemical cell using 23 wt % of Zn(ClO₄)₂, 16 wt % of LiTFSI, 7 wt % of NaClO₄ in H₂O aqueous electrolyte during a charging and discharging cycles at 2 C rate.

FIG. 27 shows a change of Coulombic efficiency of a zinc/PMPTS electrochemical cell using 23 wt % of Zn(ClO₄)₂, 16 wt % of LiTFSI, 7 wt % of NaClO₄ in H₂O aqueous electrolyte during a charging and discharging cycles at 2 C rate.

FIG. 28 shows the cyclic voltammogram (CV) of phenothiazine (PT) in 30 wt % ZnBr₂/H₂O aqueous electrolyte indicating the potentials vs. Zn/Zn²⁺ electrode whereby PT is reduced and oxidized in a reversible process at 1.25V vs. Zn/Zn²⁺.

FIG. 29 shows the CV data of t a beaker cell using PT cathode and Zn anode at 10 mV/s. The data displayed 500 cycles at 10 mV/s with no loss in current during the cycling, which indicates the remarkable stability of PT molecule in 30% ZnBr₂/H₂O electrolyte.

FIG. 30 shows the CV data of PT cathode in 30% ZnBrALO aqueous electrolyte at different scan rates of 10 mV/s, 5 mV/s, 1 mV/s. The redox current was found to be proportional to the scan rates indicating the stable reversible nature of the processes.

FIG. 31 shows the voltage profile of a zinc/phenothiazine electrochemical cell in 30% ZnBr₂/H₂O aqueous electrolyte during a charging and discharging cycle.

FIG. 32 shows a change of coulombic efficiency of a zinc/phenothiazine electrochemical cell in 30% ZnBr₂/H₂O aqueous electrolyte over a plurality of charging and discharging cycles.

FIG. 33 shows a change of discharge capacity of a zinc/phenothiazine electrochemical cell in 30% ZnBr₂/H₂O aqueous electrolyte over a plurality of charging and discharging cycles.

FIG. 34 shows the cyclic voltammogram of 2-chlorophenothiazine (CPT) in 30 wt % ZnBr₂/H₂O aqueous electrolyte vs Zn/Zn²⁺. The CPT molecule is reduced and oxidized in a reversible process at 1.30V vs Zn/Zn²⁺.

FIG. 35 shows the voltage profile of a zinc/CPT electrochemical cell in 30% ZnBr₂/H₂O aqueous electrolyte during a charging and discharging cycle.

FIG. 36 shows a change of discharge capacity of a zinc/CPT electrochemical cell in 30% ZnBr₂/H₂O aqueous electrolyte over a plurality of charging and discharging cycles.

FIG. 37 shows a change of Coulombic efficiency of a zinc/phenothiazine electrochemical cell in 30% ZnBr₂/H₂O aqueous electrolyte over a plurality of charging and discharging cycles.

FIG. 38 shows the cyclic voltammogram of sulfur-bridged bis(phenothiazine) (PT₂S) in 30 wt % ZnBr₂/H₂O aqueous electrolyte indicating the potentials vs. Zn/Zn²⁺ electrode. The PT₂S is reduced and oxidized in a reversible process at 1.30V vs Zn/Zn²⁺ at 10 mV/s scan rate.

FIG. 39 shows the voltage profile of a zinc/PT₂S electrochemical cell in 30% ZnBr₂/H₂O aqueous electrolyte during a charging and discharging cycle.

FIG. 40 shows a change of discharge capacity of a zinc/PT₂S electrochemical cell in 30% ZnBr₂/H₂O aqueous electrolyte over a plurality of charging and discharging cycles.

FIG. 41 shows a change of coulombic efficiency of a zinc/PT₂S electrochemical cell in 30% ZnBr₂/H₂O aqueous electrolyte over a plurality of charging and discharging cycles.

FIG. 42 shows a cyclic voltammogram of methylphenothiazene-bridged bis(phenothiazine) (PT₂MPT) in 30 wt % ZnBr₂/H₂O aqueous electrolyte indicating the potentials vs. Zn/Zn²⁺ electrode. The PT₂MPT is reduced and oxidized in a reversible process.

FIG. 43 shows the voltage profile of a zinc/PT₂MPT electrochemical cell in 30% ZnBr₂/H₂O aqueous electrolyte during a charging and discharging cycle.

FIG. 44 shows a change of discharge capacity of a zinc/PT₂MPT electrochemical cell in 30% ZnBr₂/H₂O aqueous electrolyte over a plurality of charging and discharging cycles.

FIG. 45 shows a change of Coulombic efficiency of a zinc/PT/MPT electrochemical cell in 30% ZnBr₂/H₂O aqueous electrolyte over a plurality of charging and discharging cycles.

FIG. 46 The top panel shows a schematic representation of an exemplary electrochemical cell including a Zn anode and a cathode comprising a tricyclic compound herein described. The bottom panel shows a schematic representation of an exemplary Pouch Housing electrochemical cell including a Zn anode and a cathode comprising a tricyclic compound herein described.

FIG. 47 shows exemplary arrangement of a plurality of electrochemical cells in a battery herein described.

FIG. 48 shows a schematic representation of an exemplary plurality of electrically connected electrochemical cells in accordance with the disclosure.

### DETAILED DESCRIPTION

Described herein are polycyclic compounds, and in particular redox active monomers, dimers and polymers, and related compositions, electrode material, electrodes, electrochemical cells, batteries, methods and systems.

The term “polycyclic compound” as used herein indicates an organic compound featuring several closed rings of atoms, primarily carbon. Exemplary polycyclic ring substructures include cycloalkanes, aromatics, and other ring types. They come in sizes of three atoms and upward, and in combinations of linkages that include tethering (such as in biaryls), fusing (edge-to-edge, such as in anthracene and steroids), links via a single atom (such as in Spiro compounds), bridged compounds, and longifolene Polycyclic compounds can be categorized according to the number of rings according to a nomenclature where they are described by specific prefixes such as bicyclic, tricyclic, tetracyclic, and additional prefixes identifiable by a skilled person.

Polycyclic compounds according to the present disclosure typically comprise at least one three ring structure as will be understandable by a skilled person.

Typically, polycyclic compounds according to the present disclosure comprise monomers, dimer, trimer or polymers featuring several closed rings of atoms, primarily carbon, comprising one or more three-ring structure also as will be understood by a skilled person.

As used herein, the term monomer refers to a single organic compound that is capable of dimerization or polymerization to form a corresponding dimer or polymer. Monomers can be molecules that bond together to form more complex structures such as polymers. Monomer can be categorized based on their sources as natural monomers, synthetic monomers, based on the respectively polarity in polar or nonpolar monomers, based on their configuration in cyclic vs linear.

Monomers can be polymerized to provide polymers comprising a plurality of monomeric unit. In particular polymerization can be performed with different monomeric unit to provide a heteropolymer or copolymer. The polymerization of one kind of monomer gives a homopolymer. Many polymers are copolymers, meaning that they are derived from two different monomers. In the case of condensation polymerizations, the ratio of comonomers is usually 1:1. For example, the formation of many nylons requires equal amounts of a dicarboxylic acid and diamine. In the case of addition polymerizations, the comonomer content is often only a few percent. For example, small amounts of 1-octene monomer are copolymerized with ethylene to give specialized polyethylene.

Polymer can be categorized based on the number of monomeric units they comprise. For example, polymers can comprise dimers trimers, tetramers as will be understood by a skilled person. In particular, the term “dimer” as used herein refers to a molecule containing two repeat same or different monomeric units. As used herein, the term “trimer” refers to a molecule containing three tricyclic compound monomeric units of the same or different structures.

In the present disclosure the term “polymer” generally refers to a molecule containing three or more repeat monomeric units.

In some embodiments, a tricyclic compound according to the present disclosure, the tricyclic compound is represented by Formula (I)

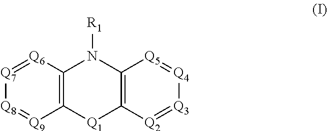

in which
Q1 is a —O—, —S— or ═NR4,
Q2 to Q9 are each independently selected from N or CR5 with the proviso that at most two of Q2 to Q5 or two of Q6 to Q9 are N, wherein R1, and R4 are independently selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
   wherein the tricyclic compound as described has a redox potential of 0.20 V to 2.0 V with reference to Zn/Zn²⁺ electrode potential under standard conditions.

As used herein, the term “redox potential” refers to an electrode potential relative to a reference electrode under standard conditions at a temperature of 298.15 K. A reference electrode can be Ag/AgCl (KCl std.) for doing all the electrochemical experiments in aqueous electrolytes. All the data in presented in this disclosures are converted to Zn/Zn²⁺ scale by adding 0.99V to the measured Ag/AgCl (KCl, stad.) electrode potential.

In some embodiments, the tricyclic compound of Formula (I) is represented by Formula (IA)

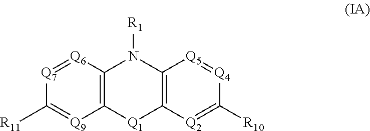

in which
Q1 is a —O—, —S— or ═NR4,
Q2, Q4 to Q7, and Q9 are each independently selected from Nor CR5 with the proviso that at most two of Q2 to Q5 or two of Q6 to Q9 are N,
wherein R1, and R4 are independently selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R10 and R11 are each independently selected from H, or any one of Formula (Ia) to Formula (9c),

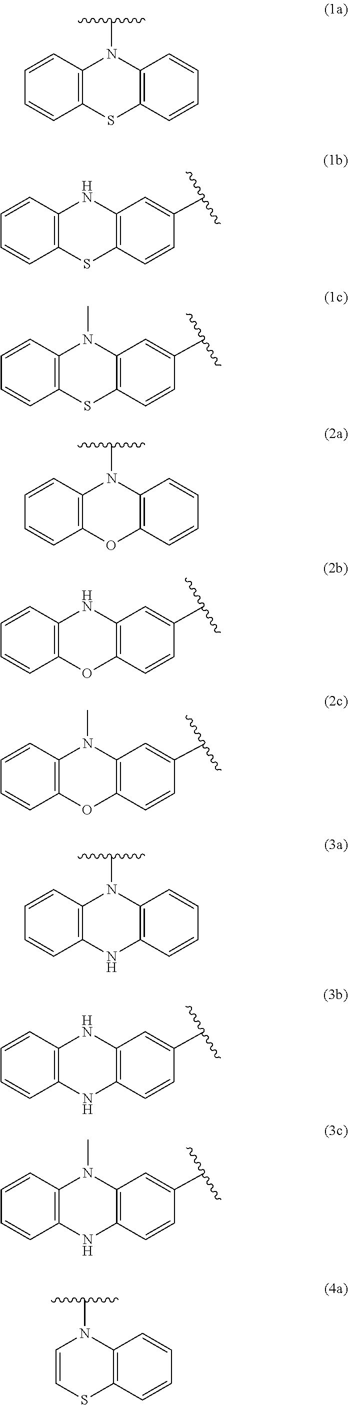

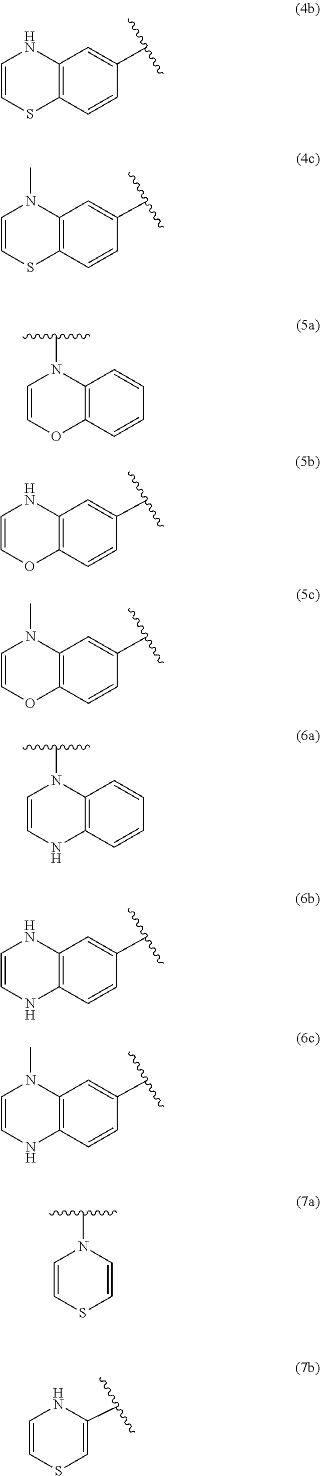

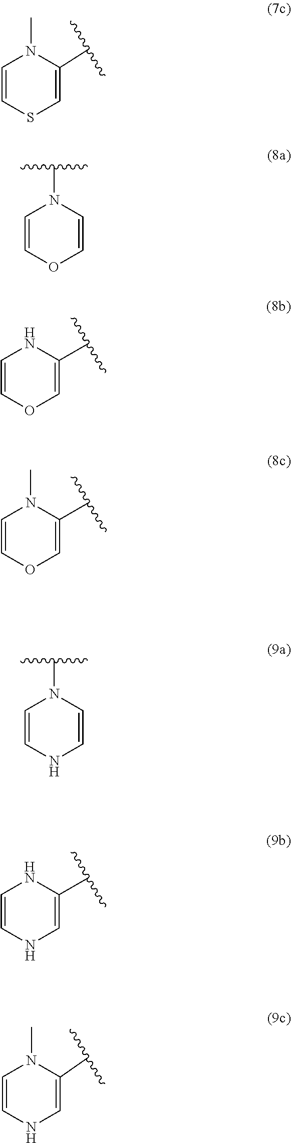

wherein the tricyclic compound as described has a redox potential of 0.20 V to 2.0 V with reference to Zn/Zn²⁺ electrode potential under standard conditions.

In some embodiments, the tricyclic compound of Formula (I) is represented by Formula (IB)

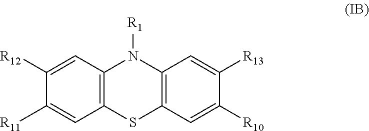

in which
wherein R1 is selected from H, or a linear or branched, C1-C4 alkyl group including methyl, ethyl, propyl, and butyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R10, R11, R12 and R13 are each independently selected from H, or any one of Formula (Ia) to Formula (9c),

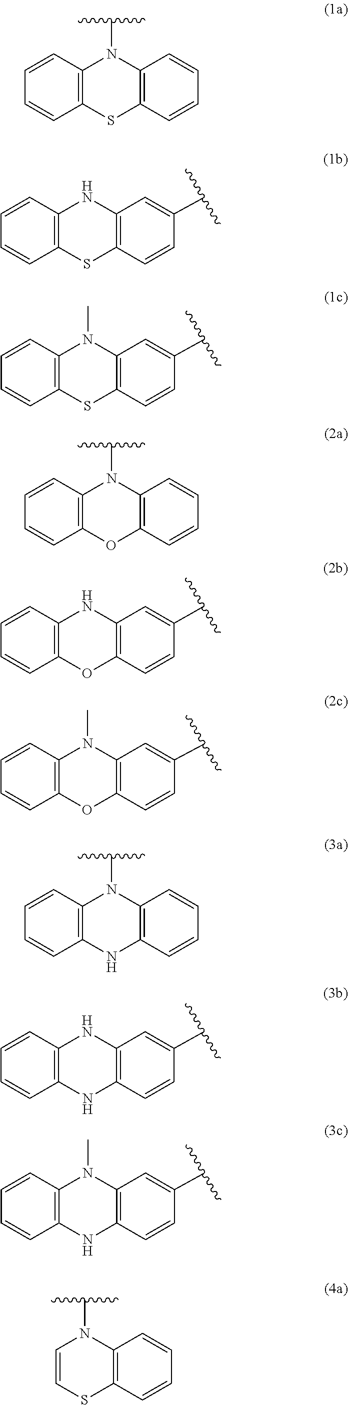

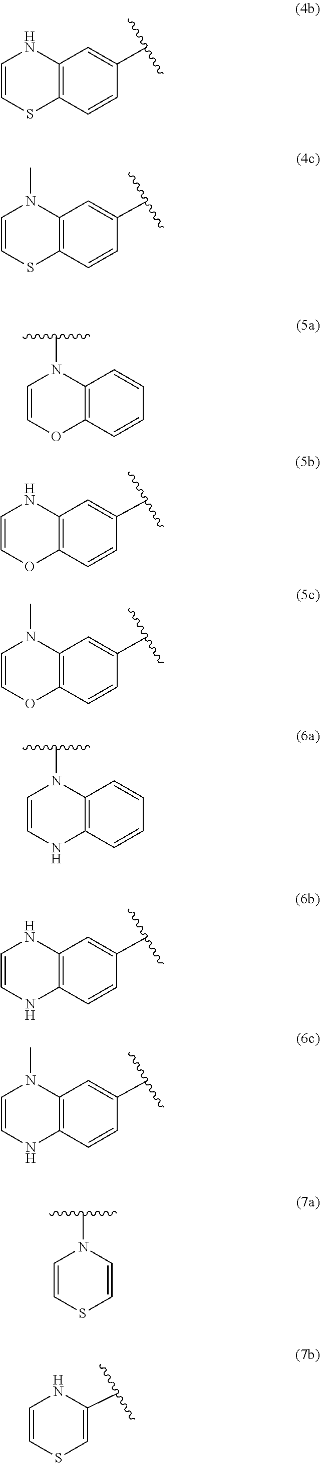

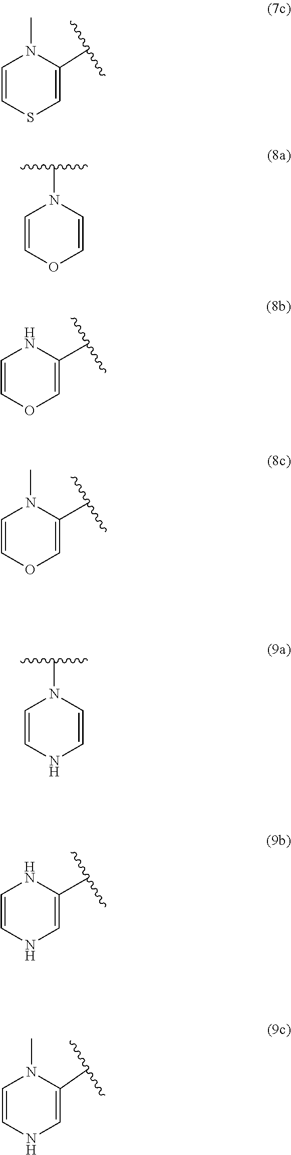

wherein the tricyclic compound as described has a redox potential of 0.20 V to 2.0 V with reference to Zn/Zn²⁺ electrode potential under standard conditions.

In some embodiments, a tricyclic compound comprising three or more three-ring structures herein described can be represented by Formula (II)

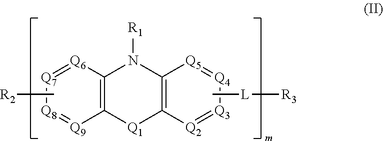

in which
Q1 is a —O—, —S— or ═NR4,
Q2 to Q9 are each independently selected from N or CR5 with the proviso that at most two of Q2 to Q5 or two of Q6 to Q9 are N,
wherein R1, and R4 are independently selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
L is null or O, S, NR1, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S, wherein L links between a ring carbon atom of any one of Q2 to Q5 to a ring carbon atom of any one of Q6 to Q9 of an adjacent monomeric moiety,
R2 and R3 are null or H,
m ranges from 3 to 10,000,
   wherein the tricyclic compound and related polymer are described have a redox potential of 0.20 V to 2.0 V with reference to Zn/Zn²⁺ electrode potential under standard conditions.

In some embodiments, a tricyclic compound comprising three or more three-ring structures of Formula (II) is represented by Formula (IIA)

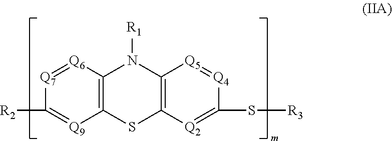

in which
Q2, Q4 to Q7 and Q9 are each independently selected from N or CR5 with the proviso that at most two of Q2 and Q4 to Q5 or two of Q6 to Q7 and Q9 are N, wherein R1 is selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
R2 and R3 are null or H,
m ranges from 3 to 10,000,
   wherein the tricyclic compound and related polymer are described have a redox potential of 0.20 V to 2.0 V with reference to Zn/Zn²⁺ electrode potential under standard conditions.

In some embodiments, a tricyclic compound comprising three or more three-ring structures of Formula (II) is represented by Formula (IIB)

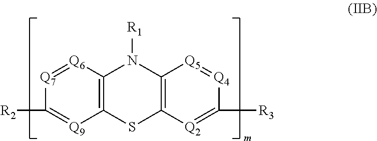

in which
Q2, Q4 to Q7 and Q9 are each independently selected from N or CR5 with the proviso that at most two of Q2 and Q4 to Q5 or two of Q6 to Q7 and Q9 are N,
wherein R1 is selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
R2 and R3 are null or H,
m ranges from 3 to 10,000,
   wherein the tricyclic compound and related polymer are described have a redox potential of 0.20 V to 2.0 V with reference to Zn/Zn²⁺ electrode potential under standard conditions.

In some embodiments, a tricyclic compound comprises two three-ring structure is described, the tricyclic compound is represented by Formula (IV)

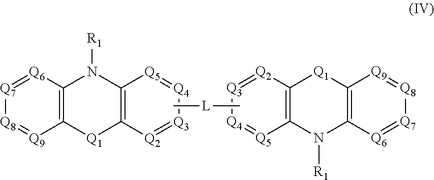

in which
Q1 is a —O—, —S— or ═NR4,
Q2 to Q5 are each independently selected from N or CR5 with the proviso that at most two of Q2 to Q5 are N and one of Q2 to Q5 is C—X wherein X is Cl, Br, or 1,
Q6 to Q9 are each independently selected from N or CR5 with the proviso that at most two of Q6 to Q9 are N,
wherein R1, and R4 are independently selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S;
wherein L is null or O, S, NR1, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N. and S, wherein L links between a ring carbon atom of any one of Q2 to Q5 to a ring carbon atom of any one of Q2 to Q5 of another monomeric moiety.

In some embodiments, a tricyclic compound comprising two three-ring structure is described, the tricyclic compound is represented by Formula (IVA) can be provided by the following reaction scheme

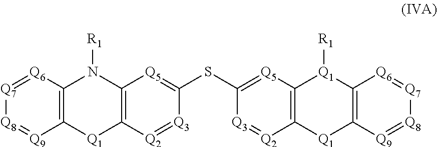

in which
Q1 is a —O—, —S— or ═NR4,
Q2, Q3 and Q5 are each independently selected from N or CR5 with the proviso that at most two of Q2, Q3 and Q5 are N,
Q6 to Q9 are each independently selected from N or CR5 with the proviso that at most two of Q6 to Q9 are N,
wherein R1, and R4 are independently selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S.

In some embodiments, a tricyclic compound comprising two three-ring structure is described, the dimer of tricyclic compound is represented by Formula (IVB)

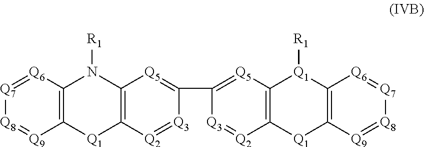

in which
Q1 is a —O—, —S— or ═NR4,
Q2, Q3 and Q5 are each independently selected from N or CR5 with the proviso that at most two of Q2, Q3 and Q5 are N,
Q6 to Q9 are each independently selected from N or CR5 with the proviso that at most two of Q6 to Q9 are N,
wherein R1, and R4 are independently selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S.

In some embodiments, a tricyclic compound comprising three or more three-ring structures is described, the tricyclic compound is represented by Formula (VII)

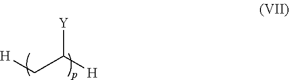

wherein p ranges from 3 to 10,000
wherein Y is selected from any one of Formula (10a), Formula (10b) and Formula (10c)

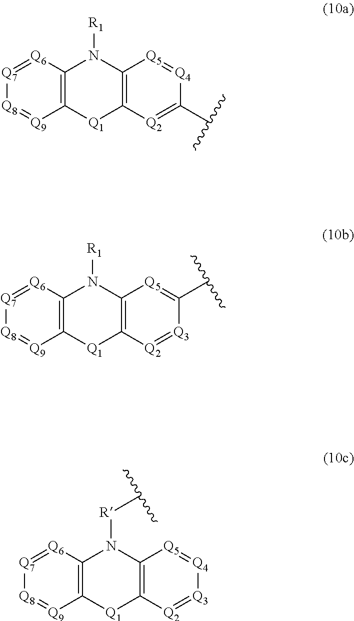

wherein
Q1 is a —O—, —S— or ═NR4,
Q2 to Q5 are each independently selected from N or CR5 with the proviso that at most two of Q2 to Q5 are N and one of Q2 to Q5 is C—X wherein X is Cl, Br, or I,
Q6 to Q9 are each independently selected from N or CR5 with the proviso that at most two of Q6 to Q9 are N,
wherein R′ is selected from a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R1, and R4 are independently selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S.

In some embodiments, any one of the tricyclic compound herein described, has a weight average molecular weight of at least 200 Dalton and/or a solubility in water of equal or less than 1.0 microgram per mL at room temperature.

As used herein, the wording “polymer” indicates an organic macromolecule of at least 500 Daltons molecular weight composed of three or more repeated subunits. For example, a subunit can be a fused three-ring structure wherein at least one of the three rings is a heterocyclic moiety. In particular, a polymer is comprised of a series of monomers resulting from a polymerization reaction. At least one of the monomers used in the polymer are redox active. Furthermore, polymers exhibit a voltage when coupled with a counter electrode. Furthermore, crosslinked polymers are able to charge and discharge over a set voltage range without immediate decomposition within an electrode.

In some embodiments any one of the tricyclic compounds can be comprised in an electrode composition is described, the electrode composition comprising one or more of the tricyclic compound of Formula (I), Formula (II), Formula (IV), or Formula (VII) herein described and/or a crosslinked polymer of Formula (II) and Formula (VII) herein described.

As used herein, a “binder” refers to a polymeric material which is non redox active under the battery working condition but enhance the adhesion of the composition to a metal surface on the electrode and maintains contact to conductive additives.

As used herein, a “conductive additive” is a solid material which when present in the electrode composition enhances the electrical conductivity of the resulting electrode composition.

In some embodiments of an electrode composition comprising one or more tricylic compound, monomers and/or polymers herein described, a binder, and a conductive additive, the binder can be 0.5-20%% by weight of one selected from the group of Polytetrafluoroethylene (PTFE), Styrene-butadiene or styrene-butadiene rubber (SBR), poly(vinylidene-fluoride) (PVDF), poly(tetrafluoroethylene), sodium carboxymethylcellulose (CMC), styrene-butadiene rubber, polyacrylic acid (PAA), polyvinyl alcohol (PVA), polyethylene glycol (PEG or PEO), polyamide imide (PAI), Polyacrylonitrile (PAN) Xanthan Gum, Gum Arabic, and Agar any combination thereof.

The electrolyte additive as described herein can include other alkali metals salts such as LiF, LiCl, LiBr, LiI, LiClO₄, LiTFSI, LiOTf, LiTFA, LiOAc, Li₂SO₄, LiNO₃, formate, NaF, NaCl, NaBr, NaI, Na₂SO₄, NaClO₄, NaOTf, NaOAc, NaTFA, KF, KCl, KBr, KI, K₂SO₄, KClO₄, KOTf, KTFSI, KOAc, KTFA, NH₄Cl, MgSO₄, organic solvents such as sulfolane, dimethyl methylphosphonate, oligomers such as polyethylene glycol (MW 100-1000 Dalton), and mixtures thereof.

In some embodiments of an electrode composition comprising one or more polycyclic compounds selected from tricyclic compound, monomers and/or polymers herein described, one or more polycyclic compounds can be present in 40 to 90% percent by weight of the total electrode composition. With increased conductivity of the active material or one or more polycyclic compounds, the amount of conductive additives in the electrode can be reduced appropriate while maintaining the same degree of the conductivity for the electrode composition. With increased stability of active material or network polymer, the amount of hinders in the electrode can be reduced accordingly physical stability of the electrode composition.

In some embodiments of an electrode composition comprising one or more tricylic compound, monomers and/or polymers herein described, a binder, and a conductive additive, the conductive additive can be 5-50% by weight of one selected from the group of Carbon Black (Acetylene Black, Super P Li, C-Nergy, Ketjen Black-300, Ketjen Black-600), Imerys (Super P, C-Nergy), carbon nanotubes (C-Nano, Tuball), graphene (xGnP Grade R, xGnP Grade H, xGnP Grade C, xGnP Grade M) and Graphite (KS-4, KS-8, KC-4, KC-8), and nickel powder or any combination thereof.

In some embodiments, the binder for the electrode composition as described herein can be selected from one of Polytetrafluoroethylene (PTFE), Styrene-butadiene or styrene-butadiene rubber (SBR), poly(vinylidene-fluoride) (PVDF), poly(tetrafluoroethylene), sodium carboxymethylcellulose (CMC), styrene-butadiene rubber, polyacrylic acid (PAA), polyvinyl alcohol (PVA), polyethylene glycol (PEG or PEO), polyamide imide (PAI), Polyacrylonitrile (PAN) Xanthan Gum, Gum Arabic, and Agar any combination thereof.

In some embodiments, the binder for the electrode composition as described herein is present in 1 to 50% by weight of the total electrode composition.

In some embodiments, the conductive additive for the electrode composition as described herein can be selected from carbon materials such as graphite, carbon black, acetylene black, and Super-P carbon, Ketjan Black as well other electrically conduction particles such as nickel powder or any combination thereof.

In some embodiments, the conductive additive for the electrode composition as described herein is present in 5 to 70% by weight of the total electrode composition.

In some embodiments, an electrode composition of the present disclosure preferably comprises PTFE and Super P, or Ketjan Black or Carbon Black.

Electrodes are preferably formed with between 40-90% active polymer material and between 3-20% binder and 10-50% conductive additive.

In some embodiments, an electrode composition is described, the electrode composition comprising any one tricyclic compound of PT (1), CPT (2), MPT (3), PPT (4), PT2S (5), PT2MPT (6), PMPTS (7), PMPT (8), PPTS (9), N-substituted PVPT (10), 2-Substituted PVMT (11), N-Substituted PAPT (12), N-phenyl substituted PSPT (13) or any combination thereof, optionally with together with a binder, and/or a conductive additive. The binder for the electrode composition as described herein can be selected from one of Polytetrafluoroethylene (PTFE), Styrene-butadiene or styrene-butadiene rubber (SBR), poly(vinylidene-fluoride) (PVDF), poly(tetrafluoroethylene), sodium carboxymethylcellulose (CMC), styrene-butadiene rubber, polyacrylic acid (PAA), polyvinyl alcohol (PVA), polyethylene glycol (PEG or PEO), polyamide imide (PAI), Polyacrylonitrile (PAN) Xanthan Gum, Gum Arabic, and Agar any combination thereof, preferably PTFE, the conductive additive for the electrode composition as described herein can be selected from carbon materials such as graphite, carbon black, acetylene black, and Super-P carbon, Ketjan Black as well other electrically conduction particles such as nickel powder or any combination thereof.

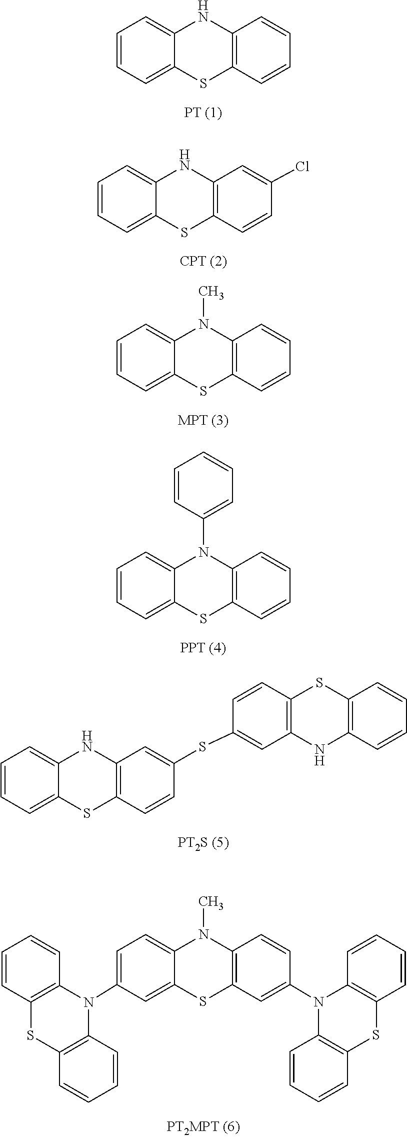 [SMILES: c1ccc2c(c1)Nc1ccccc1S2 ; Clc1ccc2c(c1)Nc1ccccc1S2 ; CN1c2ccccc2Sc2ccccc21 ; c1ccc(N2c3ccccc3Sc3ccccc32)cc1 ; c1ccc2c(c1)Nc1cc(Sc3ccc4c(c3)Nc3ccccc3S4)ccc1S2 ; CN1c2ccc(N3c4ccccc4Sc4ccccc43)cc2Sc2cc(N3c4ccccc4Sc4ccccc43)ccc21]

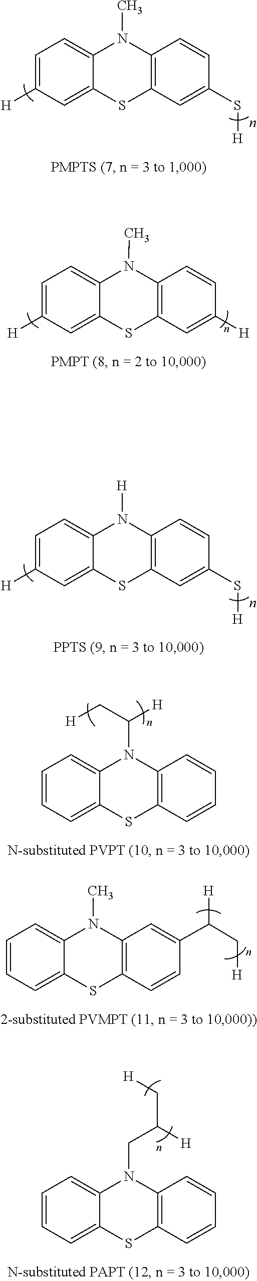

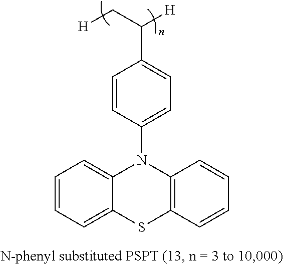

In embodiments herein described, polycyclic compound of the present disclosure can be incorporated into functional electrodes by mixing with suitable binder and conductive additive. Mixing methods include planetary mixing and high shear mixing.

Electrode formation methods include drop casting, doctor blade casting, spin coating, comma-roll coating and extrusion. In some embodiments, the composition of electrodes may vary from 30-100 wt % active material, 5-70 wt % conductive additive and 1-20 wt % binder with the total wt % of all species summing to 100%.

After mixing and coating of such electrodes, the electrodes are subjected to pressure through calendaring, followed by heating at temperatures above 50° C. Calendaring may be achieved using a heated or unheated roller.

In some embodiments, the electrode material herein described is provided in form electrodes in an electrochemical cell herein described.

As used herein, an “electrochemical cell” refers to a device capable of generating electrical energy by chemical reaction, or a device capable of using electrical energy to drive a chemical reaction, or both.

The electrochemical cells which generate an electric current are called voltaic cells or galvanic cells and those that generate chemical reactions, via electrolysis for example, are called electrolytic cells.

In particular voltaic cell (galvanic cell) is an electrochemical cell that generates electrical energy through redox (reduction-oxidation) reactions in the cell. An electrochemical cell can also use externally applied electrical energy to drive a redox reaction within the cell, referred to as an electrolytic cell. A fuel cell is an electrochemical cell that generates electrical energy from a fuel through electrochemical reaction of hydrogen with an oxidizing agent.

A voltaic cell or a redox generating electrochemical cell can include a permeable barrier between the two electrodes that allow anions and/or cations to pass from the electrolyte in contact with one electrode to the electrolyte in contact with the other electrode.

As used herein, “electrode” refers an electrically conductive material that makes contact with a non-conductive element. In the case of an electrochemical cell, the non-conductive element is an electrolyte where the chemical reactions occur. The two types of electrodes in cell are the anode and cathode. The anode is the electrode where electrons leave the electrochemical cell and where oxidation occurs. The cathode is the electrode where electrons enter the cell and where reduction occurs. By convention, anodes are considered “negative” and cathodes are considered “positive” when producing electrical energy. When the cell is using electrical energy to drive a reaction (e.g. when a rechargeable battery is charging), then the cathode is negative with respect to the anode's polarity and the convention is usually (but not always) reversed. A cell can change between energy producing (voltaic) and redox producing (electrolytic) by changing the externally applied voltage between the electrodes (changing the direction of the current through the cell).

An “electric current” or “electrical current” by the sense of the description can be described as a flow of positive charges or as an equal flow of negative charges in the opposite direction. Electrical current, by convention, goes from cathode to anode (the opposite of the flow of electrons) outside the cell, regardless of method of operation (voltaic vs. electrolytic).

The electrochemical cell as described herein can contain a cathode on a metal substrate with current collector and an anode on a metal substrate with current collector which are separated by a semipermeable insulative membrane. The cell contains an aqueous salt solution that conducts ions. These components are placed within a container. Any of the cathode or anode can comprise the redox active composition as described herein.

In embodiments herein described the electrochemical cell comprising a zinc anode, a cathode, current collectors, external housing, a separator and an aqueous electrolyte, wherein the cathode electrode comprises the tricylic compound of Formula (I), Formula (II), Formula (IV), or Formula (VII) herein described and/or a crosslinked polymer of Formula (II) and Formula (VII) herein described.

As used herein, “electrolyte” refers to a liquid or mixture of liquid and solid that contains at least a cation and a counterion for conducting ions during an electrochemical reaction in an electrochemical cell. In some embodiments as described herein, the cation of the electrolyte can be Zn ion, optionally in combination with one or more other cations as described herein.

In particular in embodiments herein described the aqueous electrolyte comprise a salt having a cation selected from Li⁺, Na⁺, K⁺, NH₄⁺, Mg²⁺. Zn²⁺, and an anion counterion selected from F⁻, Cl⁻, Br⁻, I⁻, SO₄²⁻, ClO₄⁻, OAc⁻, TFSI⁻, OTf⁻, TFA⁻, HCO₂⁻, or any combination thereof.

In embodiments herein described, electrolyte formulations are typically used at pH from 2-10 by dissolving Lewis acidic zinc salts in water at various combinations and concentrations including zinc dendrite formation and improve coulombic efficiency. Zinc is an amphoteric metal; it can react with OH⁻ and H⁺. At lower pH it is susceptible to react with acidic electrolytes and cause severe hydrogen evolution reaction (HER). Present invention addresses HER issue by adding additives to the electrolytes. In some cases, by using other salts, such as, but not limited to, LiTFSI, or by using organic solvents such as, but not limited to, sulfolane.

In particular, in some embodiments the electrolyte formulations comprise one or more Lewis acidic zinc salts such as Zn₂SO₄, Zn(OCl₄)₂, Zn(NO₃)₂, ZnF₂, ZnCl₂, ZnBr₂, ZnI₂, Zn(OAc)₂, Zn(OTf)₂, Zn(TFSI)₂, and/or Zn(BF₄)₂ alone or in combinations of other alkali metals salts such as LiF, LiCl, LiBr, LiI, LiClO₄, LiTFSI, LiOTf, LiTFA, LiOAc, Li₂SO₄, LiNO₃, Li-formate, NaF, NaCl, NaBr, NaI, Na₂SO₄, NaClO₄, NaOTf, NaOAc, NaTFA, KF, KCl, KBr, KI, K₂SO₄, KClO₄, KOTf, KTFSI, KOAc, KTFA, NH₄Cl, MgSO₄.

In some embodiments the electrolyte formulations comprise Lewis acidic zinc salts at concentrations ranging from 0.01M to 30M (0.01 wt % to 75 wt %) depending on the salt and their combinations used.

In some embodiments the pH of the electrolyte formulation can be adjusted to 4-8 by adding LiOH and/or NaOH, and/or KOH. In some embodiments, the organic solvents such as sulfolane, polyethylene glycol (MW 100 Da-1000 Dalton), CMC, polyethylene oxide (PEO, MW 100,000-1000,000 Dalton), dimethyl methyl phosphonate were added in water in various weight ratios ranging from 1 wt % to 75 wt %.

Electrochemical cells herein described can minimize irreversibility, minimize dendrite growth during zinc plating/stripping and have high Coulombic efficiency.

In some embodiments, an electrochemical cell is described, the electrochemical cell comprising a zinc anode, a cathode, current collectors, external housing, a separator and an aqueous electrolyte, wherein the cathode electrode comprises any one tricyclic compound of PT (1), CPT (2), MPT (3), PPT (4), PT₂S (5), PT₂MPT (6), PMPTS (7), PMPT (8), PPTS (9), N-substituted PVPT (10), 2-Substituted PVMT (11), N-Substituted PAPT (12), N-phenyl substituted PSPT (13) or any combination thereof, optionally with together with a binder, and/or a conductive additive.

In some embodiments, an electrochemical cell is described, the electrochemical cell comprising a zinc anode, a cathode, current collectors, external housing, a separator and an aqueous electrolyte, wherein the cathode electrode comprises any one of the tricylic compound of Formula (I), Formula (II), Formula (IV), or Formula (VII) herein described and/or a crosslinked polymer of Formula (II) and Formula (VII) or any combination thereof herein described,

In some embodiments, an electrochemical cell is described, the electrochemical cell comprising a zinc anode, a cathode, current collectors, external housing, a separator and an aqueous electrolyte, wherein the cathode electrode comprises any one tricyclic compound of PT (1), CPT (2), MPT (3), PPT (4), PT2S (5), PT2MPT (6), PMPTS (7), PMPT (8), PPTS (9), N-substituted PVPT (10), 2-Substituted PVMT (11), N-Substituted PAPT (12), N-phenyl substituted PSPT (13) or any combination thereof, optionally with together with a binder, and/or a conductive additive, wherein the aqueous electrolyte comprise a salt selected from Zn₂SO₄, Zn(OCl₄)₂, Zn(NO₃)₂, ZnF₂, ZnCl₂, ZnBr₂, ZnI₂, Zn(OAc)₂, Zn(OTf)₂, Zn(TFSI)₂, Zn(BF₄)₂, optionally in combination with LiF, LiCl, LiBr, LiI, LiClO₄, LiTFSI, LiOTf, LiTFA, LiOAc, Li₂SO₄, LiNO₃, Li-formate, NaF, NaCl, NaBr, NaI, Na₂SO₄, NaClO₄, NaOTf, NaOAc, NaTFA, KF, KCl, KBr, KI, K₂SO₄, KClO₄, KOTf, KTFSI, KOAc, KTFA, NH₄Cl, MgSO₄, wherein concentration of each salt is present at a concentration equal to or greater than 0.01M or 0.01 wt % and the total concentration in the electrolyte is equal to or less than 30 M or 75 wt %.

In some embodiments, an electrochemical cell is described, the electrochemical cell comprising a zinc anode, a cathode, current collectors, external housing, a separator and an aqueous electrolyte, wherein the cathode electrode comprises any one tricyclic compound of PT (1), CPT (2), MPT (3), PPT (4), PT₂S (5), PT₂MPT (6), PMPTS (7), PMPT (8), PPTS (9), N-substituted PVPT (10), 2-Substituted PVMT (11), N-Substituted PAPT (12), N-phenyl substituted PSPT (13) or any combination thereof, optionally with together with a binder, and/or a conductive additive, wherein the aqueous electrolyte comprise a salt selected from Zn(OTf)₂ wherein concentration of Zn(OTf)₂ is present at a concentration ranging from 1 M to 5 M.

In some embodiments, an electrochemical cell is described, the electrochemical cell comprising a zinc anode, a cathode, current collectors, external housing, a separator and an aqueous electrolyte, wherein the cathode electrode comprises any one tricyclic compound of PT (1), CPT (2), MPT (3), PPT (4), PT₂S (5), PT₂MPT (6), PMPTS (7), PMPT (8), PPTS (9), N-substituted PVPT (10), 2-Substituted PVMT (11), N-Substituted PAPT (12), N-phenyl substituted PSPT (13) or any combination thereof, optionally with together with a binder, and/or a conductive additive, wherein the aqueous electrolyte comprise a salt selected from Zn(OTf)₂ wherein concentration of Zn(OTf)₂ is present at a concentration ranging from 1 M to 5 M.

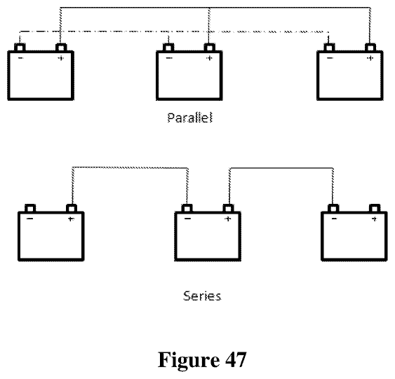

Schematic illustration of possible configuration of an electrochemical cells are illustrated in FIG. 47.

In particular FIG. 47 top panel shows an exemplary electrochemical cell including an anode, a cathode and an electrolyte disposed between the anode and cathode with an optional permeable barrier dividing the electrolyte into two ionically communicative portions. FIG. 47 bottom panel shows an exemplary electrochemical cell in a pouch housing including an anode, a cathode and their respective current collectors and an electrolyte disposed between the anode and cathode with an optional separator dividing the electrolyte into two ionically communicative portions. In some embodiments, of the present disclosure one or more electrochemical cells can be comprised within a battery.

As used herein, a “battery” is a device consisting of one or more electrical energy generating electrochemical cells arranged in parallel (for increased capacity) or serial (for increased voltage). Battery types include zinc-carbon, alkaline, nickel-oxyhydroxide, lithium, mercury oxide, zinc-air, Zamboni pile, silver-oxide, magnesium, nickel-cadmium, lead-acid, nickel-metal hydride, nickel-zinc, silver-zinc, lithium-iron-phosphate, lithium ion, and others as could be understood by a skilled person.

In particular, a battery according to this disclosure can include one or more electrochemical cells as described herein and may additionally include a first electrode coupled to an anode of the one or more electrochemical cells, a second electrode coupled to a cathode of the one or more electrochemical cells, and a casing or housing encasing the one or more electrochemical cells.

In some embodiments a battery in the sense of disclosure consists of one or more electrochemical cells, connected either in parallel, series or series-and-parallel pattern. In some embodiments, the battery can include a plurality of electrochemical cells can be linked in series or parallel based on performance demands including voltage requirement, capacity requirement.

In some embodiments, electrochemical cell as described can be electrically connected in series to increase voltage of the battery thereof.

In some embodiments, electrochemical cell as described can be electrically connected in parallel to increase charge capacity of the battery thereof.

In some embodiments, the battery as described herein can take a shape of a pouch, prismatic, cylindrical, coin.

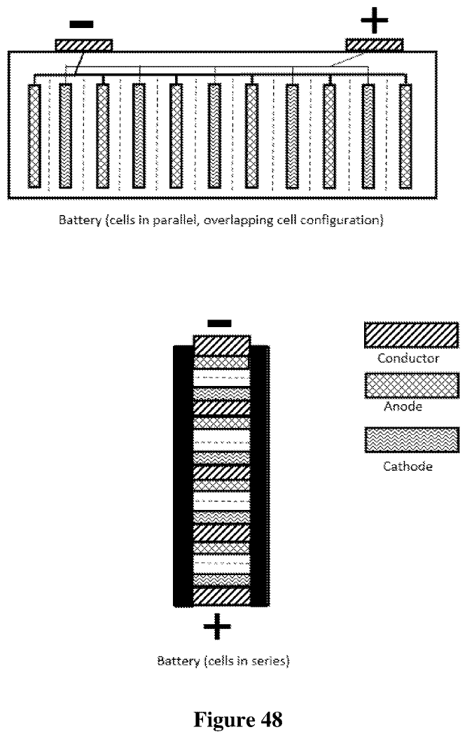

A schematic illustration of the arrangement of the electrochemical cells in a batter of the disclosure is illustrated in FIGS. 48 and 49.

FIG. 48 shows exemplary arrangement of a plurality of electrochemical cells in a battery. The top panel of FIG. 48 shows a plurality of electrically connected electrochemical cells that electrically connected in parallel, whereas the bottom panel of FIG. 48 shows a plurality of electrically connected electrochemical cells that electrically connected in series. A battery of three cells connected in parallel has a capacity of three times that of the individual cell. A battery of three cells connected in series has a voltage of three times that of the individual cell.

The top panel of FIG. 49 shows a plurality of electrically connected electrochemical cells that electrically connected in parallel in an overlapping configuration, whereas the bottom panel of FIG. 49 shows a plurality of electrically connected electrochemical cells that electrically connected in series.

The battery can be configured as a primary battery, wherein the electrochemical reaction between the anode and cathode is substantially irreversible or as a secondary battery, wherein the electrochemical reactions between the anode and cathode are substantially reversible.

Battery comprising network polymer and electrochemical cells of the disclosure are long life battery. A used herein, a long life for a battery indicates a battery that can charge/discharge for over 1,000 cycles, while retaining 70% of charge capacity. In some embodiments, a battery as described herein can have a life-time of at least four years. In some embodiments, a battery as described herein can have charge/discharge for over 1,200 cycles, while retaining 70% of charge capacity JU: we don't have the battery cycled 1200 cycles. We have only 567 cycles. We can delete this whole paragraph.

Battery comprising network polymer and electrochemical cells of the disclosure are long life battery. A used herein, a long life for a battery indicates a battery that can charge/discharge for over 1,000 cycles, while retaining 70% of charge capacity. In some embodiments, a battery as described herein can have a life-time of at least four years. In some embodiments, a battery as described herein can have charge/discharge for over 1,200 cycles, while retaining 70% of charge capacity.

Polycyclic compounds herein described to be included in electrochemical cells and batteries in accordance with the present disclosure can be provided according to methods identifiable by a skilled person upon reading of the present disclosure

In some embodiment a method is described for making a dimer of tricyclic compound, the method comprising
providing a tricyclic monomer of Formula (III)

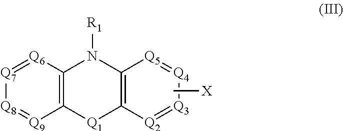

in which
Q1 is a —O—, —S— or ═NR4,
Q2 to Q5 are each independently selected from N or CR5 with the proviso that at most two of Q2 to Q5 are N and one of Q2 to Q5 is C—X wherein X is Cl, Br, or I,
Q6 to Q9 are each independently selected from N or CR5 with the proviso that at most two of Q6 to Q9 are N,
wherein R1, and R4 are independently selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S;
contacting the tricyclic monomer of Formula (III) and CuI or a suitable salt of linker L for a time and under conditions to allow reaction of the tricyclic monomer of Formula (III) with CuI or the salt of linker L to provide the dimer of Formula (IV) herein described,
wherein L is null when CuI is used or O, S, NR1, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S, wherein L links between a ring carbon atom of any one of Q2 to Q5 to a ring carbon atom of any one of Q2 to Q5 of another monomeric moiety,

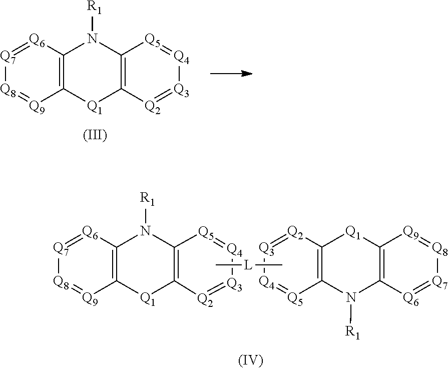

In some embodiments, the dimeric polycyclic compound of Formula (IVA) can be provided by the following reaction scheme

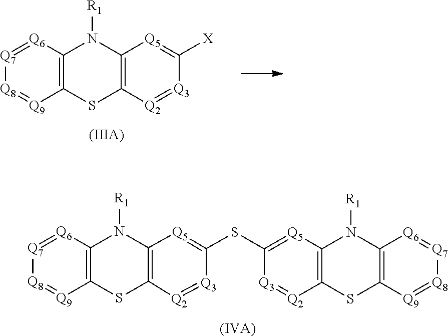

in which
Q1 is a —O—, —S— or ═NR4,
Q2, Q3 and Q5 are each independently selected from N or CR5 with the proviso that at most two of Q2 to Q5 are N and one of Q2 to Q5 is C—X wherein X is Cl, Br, or I,
Q6 to Q9 are each independently selected from N or CR5 with the proviso that at most two of Q6 to Q9 are N,
wherein R1, and R4 are independently selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S;
contacting the tricyclic monomer of Formula (IIIA) suitable salt of linker —S— for a time and under conditions to allow reaction of the tricyclic monomer of Formula (IIA) with the salt of linker —S— to provide the dimer of Formula (IVA) herein described, wherein X is a suitable substituent selected from Cl, Br or I.

In some embodiments, the dimeric polycyclic compound of Formula (IVB) can be provided by the following reaction scheme

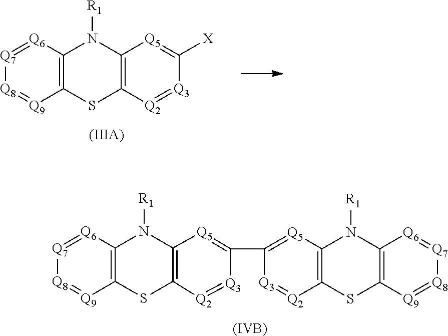

in which
Q2, Q3 and Q5 are each independently selected from N or CR5 with the proviso that at most two of Q2 to Q5 are N and one of Q2 to Q5 is C—X wherein X is Cl, Br, or I,
Q6 to Q9 are each independently selected from N or CR5 with the proviso that at most two of Q6 to Q9 are N,
wherein R1, and R4 are independently selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S;
contacting the tricyclic monomer of Formula (IIIA) suitable coupling reagent for a time and under conditions to allow C—C bond formation reaction of the tricyclic monomer of Formula (IIIA) with the coupling reagent to provide the dimer of Formula (IVB) herein described,
   wherein X is a suitable substituent selected from Cl, Br or I.

In some embodiments polycyclic compounds can be provided by a method for making a polymer of tricyclic compound, the method comprising
providing a tricyclic monomer of Formula (V)

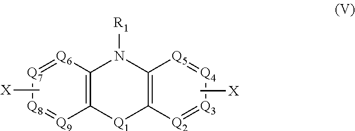

in which
Q1 is a —O—, —S— or ═NR4,
Q2 to Q9 are each independently selected from N or CR5 with the proviso that at most two of Q2 to Q5 or at most two of Q6 to Q9 are N and one of Q2 to Q5 and one of Q6 to Q9 are C—X wherein X is Cl, Br, or I,
wherein R1, and R4 are independently selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
contacting the tricyclic monomer of Formula (V) and CuI or a suitable salt of linker L for a time and under conditions to allow reaction of the tricyclic monomer of Formula (V) with CuI or the salt of linker L to provide the polymer of Formula (II) herein described,
wherein L is null when CuI is used, or O, S, NR1, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S, wherein L links between a ring carbon atom of any one of Q2 to Q5 to a ring carbon atom of any one of Q6 to Q9 of an adjacent monomeric moiety,
R2 and R3 are null or H,
m ranges from 3 to 10,000,

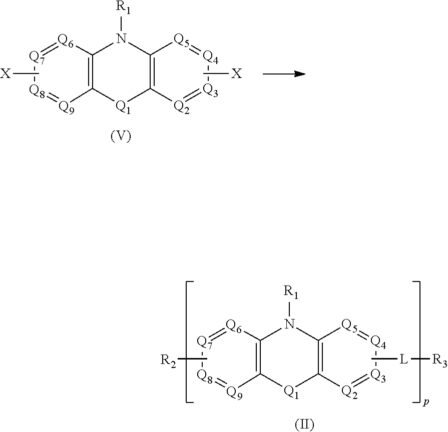

In some embodiments, a method is described for making a polymer of tricyclic compound of Formula (IIA), the method comprising
providing a tricyclic monomer of Formula (VA)

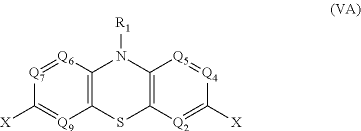

in which
Q1 is a —O—, —S— or ═NR4,
Q2, Q4, Q5 to Q7 and Q9 are each independently selected from N or CR5 with the proviso that at most two of Q2 and Q4 to Q5 or at most two of Q6 to Q7 and Q9 are N wherein X is Cl, Br, or I,
wherein R1, and R4 are independently selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
contacting the tricyclic monomer of Formula (V) with a suitable salt of linker —S— for a time and under conditions to allow reaction of the tricyclic monomer of Formula (VA) with the salt of linker —S— to provide the polymer of Formula (HA) herein described,
R2 and R3 is H,
m ranges from 3 to 10,000,

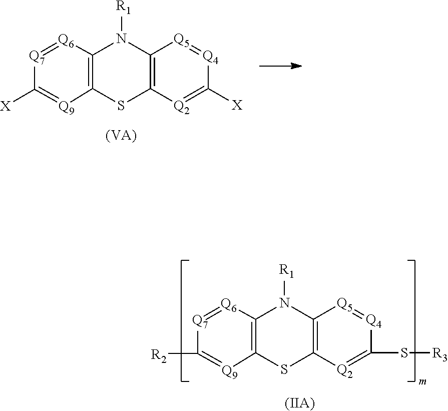

In some embodiments, a method is described for making a polymer of tricyclic compound of Formula (IIA), the method comprising
providing a tricyclic monomer of Formula (VA)

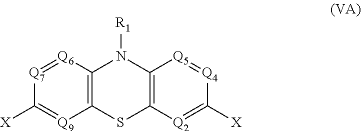

in which
Q1 is a —O—, —S— or ═NR4,
Q2, Q5 to Q7 and Q9 are each independently selected from N or CR5 with the proviso that at most two of Q2 and Q4 to Q5 or at most two of Q6 to Q7 and Q9 are N and one of Q2 and Q4 to Q5 and one of Q6 to Q7 and Q9 are C—X wherein X is Cl, Br, or I,
wherein R1, and R4 are independently selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
contacting the tricyclic monomer of Formula (V) with a suitable coupling reagent including CuI for a time and under conditions to allow C—C bond formation reaction of the tricyclic monomer of Formula (VA) to provide the polymer of Formula (IIB) herein described,
R2 and R3 is H,
m ranges from 3 to 10,000,

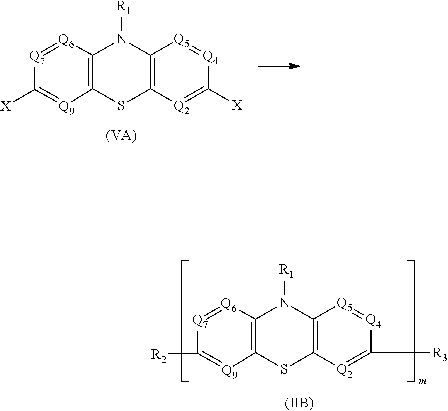

In some embodiments a tricyclic compound polymer of Formula (VII) can be manufactured by a method comprising
providing a tricyclic monomer of Formula (VI)

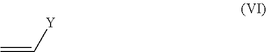

wherein Y is selected from any one of Formula (10a), Formula (10b) and Formula (10c)

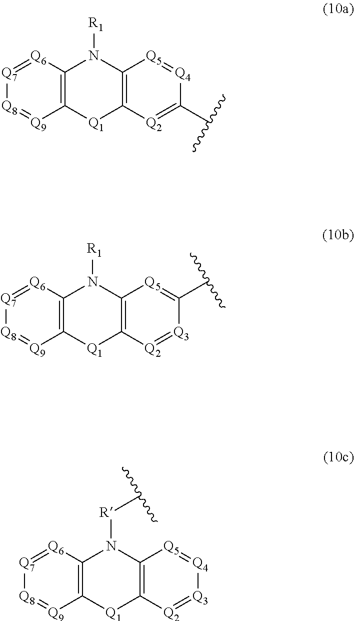

wherein
Q1 is a —O—, —S— or ═NR4,
Q2 to Q5 are each independently selected from N or CR5 with the proviso that at most two of Q2 to Q5 are N and one of Q2 to Q5 is C—X wherein X is Cl, Br, or I,
Q6 to Q9 are each independently selected from N or CR5 with the proviso that at most two of Q6 to Q9 are N,
wherein R′ is selected from a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R1, and R4 are independently selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
contacting the tricyclic monomer of Formula (VI) with a polymerization initiator or catalyst for a time and under conditions to allow polymerization of the tricyclic monomer of Formula (VI) to provide a polymer of Formula (VII) herein described,

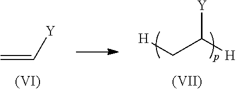

wherein p ranges from 3 to 10,000.

In some embodiments, a tricyclic compound polymer is described herein, the tricyclic compound polymer is represented by Formula (VIIA), the method comprising

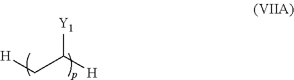

wherein Y1 is selected from any one of Formula (Ia), to Formula (3e)

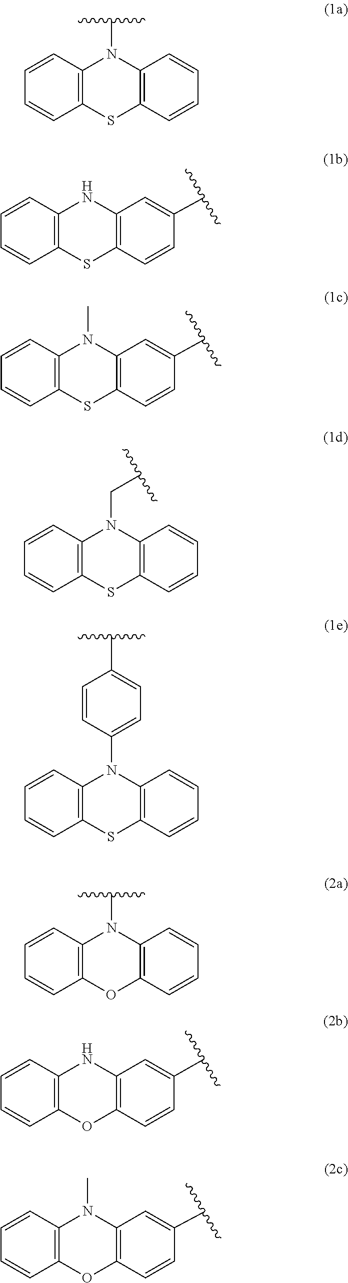

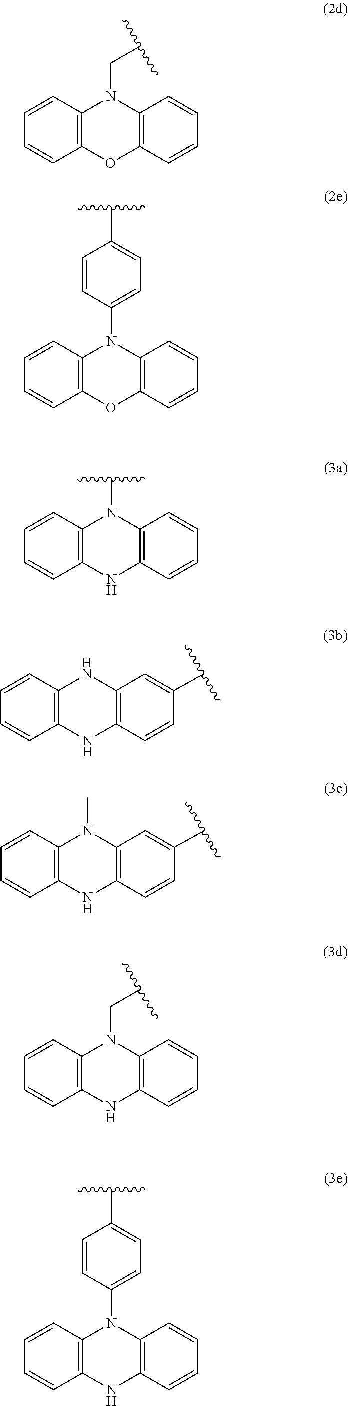

wherein p ranges from 3 to 10,000.

Synthesis of a monomer (11c) for substituent (1b) is shown in Example 16 form a commercially available starting material (11a). A person skill in the art will be able to formulate a synthetic procedure for other variants of monomer encompassed by Formula (VI).

Synthesis of a tricyclic compound (11d) corresponding to substituent (1b) was shown in Example 17. A person skill in the art will be able to formulate synthetic procedure for other variants of monomer encompassed by tricyclic compound represented by Formula (VII).

In some embodiments, a method is described for making a tricyclic compound polymer of Formula (VIIA), the method comprising
providing a tricyclic monomer of Fonnula (VIA)

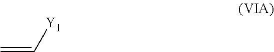

wherein Y1 is selected from any one of Formula (1a), to Formula (3e)

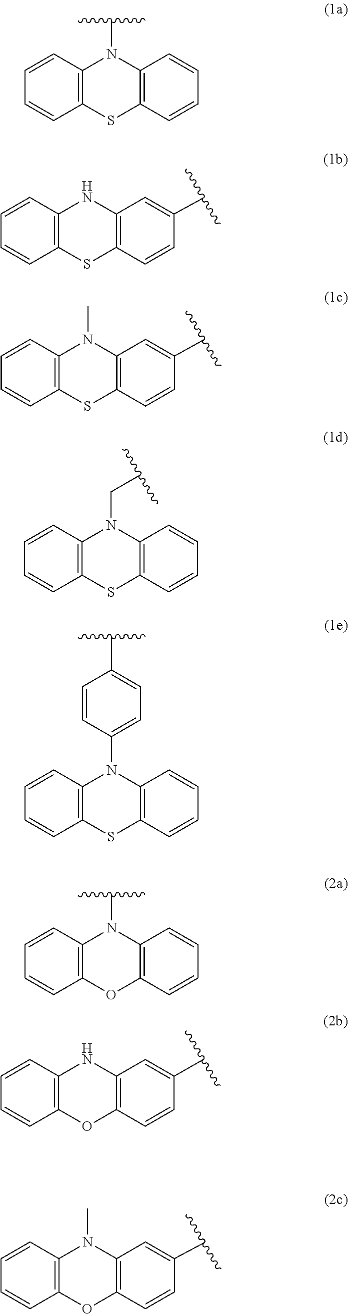

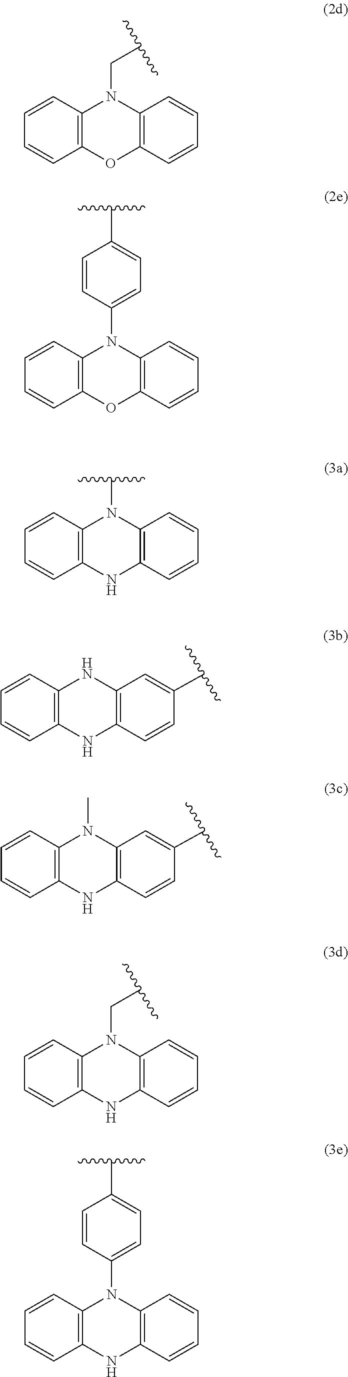

contacting the tricyclic monomer of Formula (VIA) with a polymerization initiator or catalyst for a time and under conditions to allow polymerization of the tricyclic monomer of Formula (VIA) to provide a polymer of Formula (VIIA) herein described,

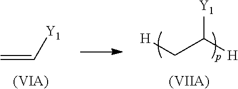

wherein p ranges from 3 to 10,000.

In some embodiments, the initiator for the polymerization of tricyclic monomer of Formula (VI) or Formula (VIA) can be selected from azoisobutylnitrile (AIBN) for photoinitiation, dicumyl peroxide for thermal initiation, and potassium persulfate for emulsion polymerizations, and other suitable initiators as known by a skilled person.

In some embodiments, the catalyst for the polymerization of tricyclic monomer of Formula (VI) or Formula (VIA) can be selected from a Ziegler-Natta catalyst comprising a combination of titanium tetrachloride (TiCl4) and diethylaluminium chloride (Al(C2H5)2Cl), a metallocene catalyst including Cp2MCl2 (M=Ti, Zr, Hf) such as titanocene dichloride, or any suitable catalyst as is known by a skilled person.

The specific chemical moiety, groups and substituents can be selected to in order to provide the desired redox activity as will be understood by a skilled person.

The term “chemical moiety” as used herein indicates an atom or group of atoms that when included in a molecule is responsible for a characteristic chemical reaction of that molecule or an atom or group of atoms that that is retained to become part of the reaction product after the reaction. A chemical moiety comprising at least one carbon atom is also indicated as organic moiety as will be understood by a skilled person.

In particular, as used here, the wording “organic moiety” refers to a carbon containing portion of an organic molecule. For example, within an organic polymer organic moieties can be formed by a distinct portion of the polymer, such as a distinct portions of a monomer that is retained in the polymer following polymerization as part of the monomeric unit of the polymer. An exemplary organic moiety is provided by a 1,5-dichloroanthraquinone or by an anthraquinone moiety retained in a network polymer as disclosed herein.

Exemplary chemical moieties in the sense of the disclosure are provided by functional groups such as hydrocarbon groups containing double or triple bonds, groups containing halogen, groups containing oxygen, groups containing nitrogen and groups containing phosphorus and sulfur all identifiable by a skilled person.

A skilled person will be able to identify the moiety that can be used in methods of the disclosure to provide the redox active polycyclic compound of the disclosure.

The term “redox active” as used herein indicates a chemical moiety (e.g. polymer or monomer or portion thereof) capable of being reversibly oxidized or reduced in an aqueous environment to produce a detectable redox potential. Redox active functional groups include but are not limited to ketones, aldehydes, and carboxylic acids.

In polycyclic compounds herein described the redox active moiety has a redox potential of 0.20 V to 2.0 V with reference to Zn/Zn2+ electrode potential under standard conditions. It is to be understood that a person of skill in the art would know that Li/Li⁺ has a potential of −3.04 V vs. SHE, a potential of a redox moiety relative to the potential of Li/Li⁺ can be converted to a potential of a redox moiety relative to SHE by subtraction of the potential vs. Li/Li⁺ by 3.04 V to give the potential vs. SHE.

Accordingly, the polycyclic compounds herein described have a charging capacity as will be understood by a skilled person. As used herein, the wording “charging capacity” is a measurement of the product of current times time of the charge that the anode material accepts until a cutoff voltage is reached. Discharging capacity is the product of current times time of the charge that the cathode material accepts until a cutoff voltage is reached.

$$q = \frac{n F}{3600 * M W}$$

where Q is the theoretical capacity,
n is the number of electrons exchanged,
F is Faraday's constant, and
MW is the molecular weight of the electroactive material.

Since in tricyclic compound herein described has a redox potential of 0.20 V to 2.0 V with reference to Zn/Zn2+ electrode potential, to increase or decrease the redox potential of a starting redox active monomeric moiety, a substituent group can be selected, based on the Hammett Sigma constant such as the constants shown in the following Table 1.

TABLE 1
Hammett Sigma Constants*

| Group | σmeta | σpara | σI | σv | π | Eₛ | MR |
|---|---|---|---|---|---|---|---|
| H | 0.00 | 0.00 | 0.00 | 0.00 | 0.00 | 0.00 | 1.03 |
| CH₃ | −0.07 | −0.17 | −0.04 | 0.52 | 0.56 | −1.24 | 5.65 |
| C₂H₅ | −0.07 | −0.15 | −0.05 | 0.56 | 1.02 | −1.31 | 10.30 |
| n-C₃H₇ | −0.07 | −0.13 | −0.03 | 0.68 | 1.55 | −1.60 | 14.96 |
| i-C₃H₇ | −0.07 | −0.15 | −0.03 | 0.76 | 1.53 | −1.71 | 14.96 |
| n-C₄H₉ | −0.08 | −0.16 | −0.04 | 0.68 | 2.13 | −1.63 | 19.61 |
| t-C₄H₉ | −0.10 | −0.20 | −0.07 | 1.24 | 1.98 | −2.78 | 19.62 |
| H₂C═CH** | 0.05 | −0.02 | 0.09 | 2.11 | 0.82 | | 10.99 |
| C₆H₅* | 0.06 | −0.01 | 0.10 | 2.15 | 1.96 | −3.82 | 25.36 |
| CH₂Cl | 0.11 | 0.12 | 0.15 | 0.60 | 0.17 | −1.48 | 10.49 |
| CF₃ | 0.43 | 0.54 | 0.42 | 0.91 | 0.88 | −2.40 | 5.02 |
| CN | 0.56 | 0.66 | 0.53 | 0.40 | −0.57 | −0.51 | 6.33 |
| CHO | 0.35 | 0.42 | 0.25 | | −0.65 | | 6.88 |
| COCH₃ | 0.38 | 0.50 | 0.29 | 0.50 | −0.55 | | 11.18 |
| CO₂H** | 0.37 | 0.45 | 0.39 | 1.45 | −0.32 | | 6.93 |
| Si(CH₃)₃ | −0.04 | −0.07 | −0.13 | 1.40 | 2.59 | | 24.96 |
| F | 0.34 | 0.06 | 0.52 | 0.27 | 0.14 | −0.46 | 0.92 |
| Cl | 0.37 | 0.23 | 0.47 | 0.55 | 0.71 | −0.97 | 6.03 |
| Br | 0.39 | 0.23 | 0.50 | 0.65 | 0.86 | −1.16 | 8.88 |
| I | 0.35 | 0.18 | 0.39 | 0.78 | 1.12 | −1.40 | 13.94 |
| OH | 0.12 | −0.37 | 0.29 | 0.32 | −0.67 | −0.55 | 2.85 |
| OCH₃ | 0.12 | −0.27 | 0.27 | 0.36 | −0.02 | −0.55 | 7.87 |
| OCH₂CH₃ | 0.10 | −0.24 | 0.27 | 0.48 | 0.38 | | 12.47 |
| SH | 0.25 | 0.15 | 0.26 | 0.60 | 0.39 | −1.07 | 9.22 |
| SCH₃ | 0.15 | 0.00 | 0.23 | 0.64 | 0.61 | −1.07 | 13.82 |
| NO₂** | 0.71 | 0.78 | 0.76 | 1.39 | −0.28 | −2.52 | 7.36 |
| NO | 0.62 | 0.91 | 0.37 | | −0.12 | | 5.20 |
| NH₂ | −0.16 | −0.66 | 0.12 | | −1.23 | −0.61 | 5.42 |
| NHCHO | 0.19 | 0.00 | 0.27 | | −0.98 | | 10.31 |
| NHCOCH₃ | 0.07 | −0.15 | 0.26 | | −0.37 | | 16.53 |
| N(CH₃)₂ | −0.15 | −0.83 | 0.06 | 0.43 | 0.18 | | 15.55 |
| N(CH₃)₃⁺ | 0.88 | 0.82 | 0.93 | 1.22 | −5.96 | | 21.20 |

*σmeta, σpara = Hammett constants; σI = inductive sigma constant; σv = Charton's v (size) values; p = hydrophobicity parameter; Es = Taft size parameter; MR = molar refractivity (polarizability) parameter.
**indicates that the group is in the most sterically hindered conformation.

For example, to increase redox potential of a starting redox active monomeric moiety having an aromatic ring, a CN or a CF₃ group can be comprised as can be comprised in view of the related Hammett Sigma Constant. Additional modifications to increase or decrease the redox potential of a starting moiety will be understood by a skilled person upon reading of the present disclosure.

In summary, electrode materials including tricyclic compounds redox-active species are described here, alongside functional electrodes incorporating such species and electrochemical cells and batteries including such electrodes. In certain embodiments, the electrode material described herein exhibits high mechanical strength and excellent processability into a functional electrode due to its unique composition. Advantageously, in certain embodiments the electrode supports battery charging and recharging for hundreds of cycles without material loss, due to the insoluble nature and, stability of these tricyclic compounds and polymers in the Lewis acidic zinc electrolytes used.

Further details concerning the tricyclic compounds, and related composition electrochemical cells, batteries methods and systems including generally manufacturing and packaging of the tricyclic compound compositions, electrochemical cells and/or the batter, can be identified by the person skilled in the art upon reading of the present disclosure.

### EXAMPLES

The tricyclic compounds including monomer, dimer, and polymers, and related composition, Zn electrochemical cells, batteries methods and systems herein described are further illustrated in the following examples, which are provided by way of illustration and are not intended to be limiting.

A skilled person will be able to identify additional tricyclic compounds and related composition, zinc electrochemical cells, batteries methods and systems in view of the content of the present disclosure. The following specific examples are given to illustrate the practice of the invention, but are not to be considered as limiting the invention in any way.

In particular, exemplary redox active organic molecules, metals and related electrodes, devices, compositions, methods and systems, are described in connection with specific experimental tests and procedures. A skilled person will be able to understand and identify the modifications required to adapt the results illustrated in the exemplary embodiments of this sections to additional embodiments of the tricyclic compounds, and related electrodes, devices, compositions, methods and systems in accordance with the present disclosure.

The following materials and methods can be used for all compounds and their precursors exemplified herein.

All zinc system electrochemical measurements were taken using a Biologic SP-150 potentiostat, a Neware tester or an Arbin tester.

A beaker-type cell (or beaker cell as used herein interchangeably) was used here for measurement of all cyclic voltammetry of the tricyclic compounds. The beaker cell includes glass container holding an electrolyte, a cathode tricyclic material mixing with conductive carbon and additive is used as the working electrode (WE), a Ag/AgCl (KCl satd.) is used as reference electrode, and Pt wire is used as counter electrode (CE).

All polymers of the present disclosure were filtered and washed with deionized water and acetone until solvents passing through the filter were clear.

Zinc Anode Formulation: Zinc anodes are comprised of zinc foil (McMaster, thickness 0.02″), or zinc oxide (99.9%, ZOCHEM INC., Canada), or zinc powder (99.9%, EverZinc Group SA, Belgium), or the mixture of zinc oxide and zinc powder, where zinc oxide composition ranges from 10-90 wt %, or the mixture of zinc powder and conducting carbon (Super P C65, Super P, IMERYS Graphite and Carbon), where conducting carbon composition ranges from 5-50 wt %.

To help with cell formation as well as electrode conductivity, different additives are also used, including, but not limited to, bismuth oxide, carbon black powders, graphite, carbon fibers, graphene, carbon nanofibers, and carbon fibers. To limit zinc anode solubility and improve cycle life, certain additives such as, but not limited to, potassium fluoride, calcium oxide, calcium hydroxide, and calcium zincate were used.

To stabilize the corrosion, added zinc powder alloyed with bismuth, indium, or tin. Different metal oxides and metal hydroxides, such as ZnO, In₂O₃, In(OH)₃, In₂SO₃, SnO and Bi₂O₃ were also added. Various surfactants, such as but not limited to, Triton, Tergitol, PEG etc. were used to suppress the corrosion of the zinc anode.

The zinc anode slurry was coated on to the substrate to hold active material. The substrate can be in the form of foil, perforated foil, foam, or mesh. The material of the substrate can be zinc, copper, nickel, titanium, or stainless steel. Plating the substrate with a thin layer of tin or zinc can help with corrosion and shelf life of the battery.

To enhance adhesion, cohesion, and structural features of the anode, carbon fiber, zirconium fiber, alumina fiber or silicon fiber have all been incorporated into the anode formulation.

To hold the anode to the substrate a form of binder is used. Preferred binder can be PTFE, SBR, PVDF, HEC, CMC, Arabic Gum, xanthan gum, HPMC, and chitosan.

The anode can be applied using wet process by mixing all the active materials and additives and binders with water then coat or used as a dry powder and pressed onto aforementioned substrates.

### Example 1: Features of Polycyclic Electrochemical Cell

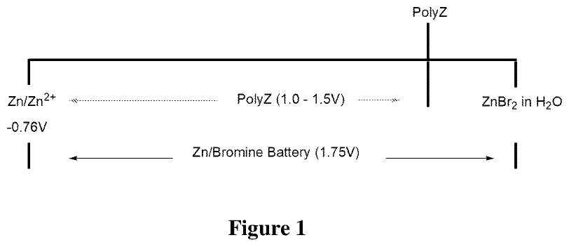

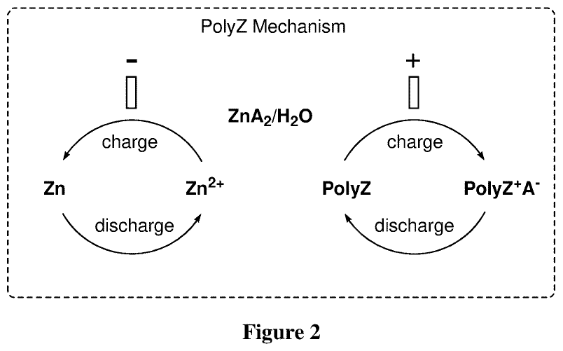

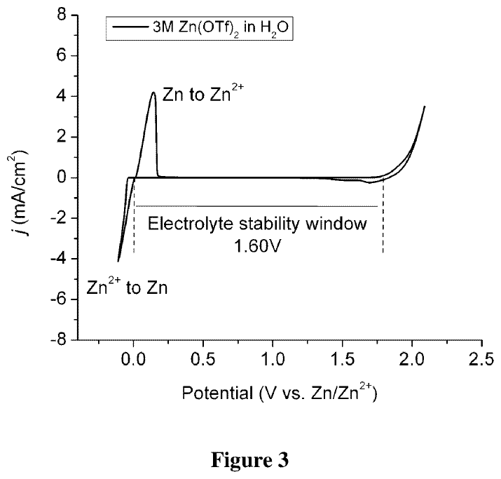

General features of the polycyclic electrodes herein described are illustrated in the schematics of FIG. 1 and FIG. 2, and in the chart FIG. 3.

In particular, FIG. 1 schematically show advantages of an electrochemical cell according to the present disclosure compared to an electrochemical cell comprising a Zn/Br₂ cathode. In particular the illustration of FIG. 1 show that an electrochemical cell according to the disclosure allows achievement of (i) Complete elimination of toxic Br₂ or Cl₂ gas formation, (ii) Complete elimination of soluble Br₃⁻ and higher bromide/interhalogen compound formation, (iii) No consumption of electrolyte salt due to side reaction, (iv) Elimination of O₂ gas formation at cathode, (v) Improve Zn cycling by adding organic additives and solvents.

The charges and discharge for the Zn anode and the charge and discharge of a cathode formed by a tricyclic compound of the present disclosure in aqueous electrolyte, is schematically shown in FIG. 2.

The electrolyte stability window of 1.6 V for a Zn anode suitable to be used in an electrochemical cell of the disclosure in water in 3M Zn(OTf)₂ is shown in the FIG. 3 with a carbon cathode.

### Example 2: Preparation of Electrolytes

In one exemplary electrolyte of 3M Zn(OTf)2 in water, 10.91 g of Zn(OTf)2 was added in water in volumetric flask to make the final volume at 10 mL. The pH of the electrolyte solution was measured with a pH meter to be ˜3.

In another exemplary electrolyte of 23 wt % of Zn(ClO4)2, 16 wt % of LiTFSI, 7 wt % of NaClO4 in H2O Electrolyte, 2.3 g of Zn(ClO4)2, 1.6 g LiTFSI and 0.7 g of NaClO4 were each added in vial and then added 10 g H2O to make a homogeneous electrolyte solution. The pH of the electrolyte solution was measured with a pH meter to be ˜4.

In yet another exemplary electrolyte of 3M Zn(OTf)2 4M LiTFSI in H2O, 9.5 g of Zn(OTf)2 and 11.5 g LiTFSI were each added in a volumetric flask and then added H2O to adjust the volume at 10 mL. The pH of the electrolyte was measured with a pH meter to be 2. In another exemplary electrolyte of 30% wt % ZnBr2 in H2O, 30 g of anhydrous ZnBr2 was added to 100 g of water and stirred at room temperature until the complete dissolution of ZnBr2 salt to make a 30% wt % ZnBr2 aqueous electrolyte.

In still another exemplary electrolyte of 10M ZnCl2+5M NaClO4 in H2O, 13.63 g of ZnCl2 and 6.67 g of NaClO4 were each added in a volumetric flask and then added water to adjust the volume at 10 mL to make an aqueous electrolyte of 10M ZnCl2+5M NaClO4 in H2O.

FIG. 2 shows a working mechanism for PolyZ Zn battery wherein the tricyclic compounds are not soluble and remain stable in the aqueous electrolyte during electrochemical cycling.

The wide electrochemical stability window of water and high reversibility of plating and stripping of zinc were achieved by formulating the electrolytes with the combinations of salts and solvent described herein.

### Example 3: Voltage Window of Aqueous Electrolyte

In this example, a series of cyclic voltammetry (CV) experiments was performed to determine the stability window of aqueous electrolytes. A 3M Zn(OTf)₂/H₂O was prepared using Zn(OTf)₂ (98%) as received from Sigma and DI water (purified by NALCO resin, resistivity >25,000 mega ohm). The cyclic voltammetry (CV) experiment was done using a three-electrode cell. A Biologic SP-150 Potentiostat was used to record the electrochemical data. A glassy carbon (0.5 cm²) was used the working electrode, a Pt wire was used as reference electrode, and an Ag/AgCl (KCl, stad.) was used as reference electrode. The CV data at 10 mV/s scan rate for 3M Zn(OTf)₂/H₂O electrolyte is presented in FIG. 3. The potential (V) was converted to Zn/Zn²⁺ by adding 0.99V to the data obtained from Ag/AgCl (KCl, stad.) reference electrode. The stability window of electrolyte was found to be >1.6V, which is wide enough for the electrochemical cells disclosing in this disclosure. The zinc stripping and platting was observed at 0.00V vs. Zn/Zn²⁺, as expected, as shown in FIG. 3.

### Example 4: Cyclic Voltammetry (CV) Experiment of Phenothiazine (PT)

In this example, a CV experiment of phenothiazine (PT) was performed in 3M Zn(OTf)₂/H₂O electrolyte. The electrolyte was prepared the same way as described in Example 3. The composition of the active material (PT) and conducting carbon and binder for this cathode was 43:43:16 wt %, respectively. The cathode was prepared as follows: the 43 wt % PT (Sigma, 98%) and 43 wt % Super P (Super P, IMERYS Graphite and Carbon) were well-mixed using a mortar and pestle. A 50 wt % of H₂O:EtOH (1:1 by vol) was added into the mixture, and then added 16 wt % of PTFE (60% solution in H₂O, FLUOROGISTX).

The overall mixture was mixed with the Thinky centrifuged instrument at 2000 rpm for 1 minute. The mixture was dried at 80° C. for overnight to remove H₂O and EtOH, completely. The mixture was then rolled using a hand roller into a free-standing cathode electrode. The free-standing electrode was dried at 80° C. for overnight and used in all cyclic voltammetry (CV) experiments. The three electrode cell described in Example 3, was used in this experiment with PT:SP:PTFE (43:43:16) as working electrode (0.25 cm²).

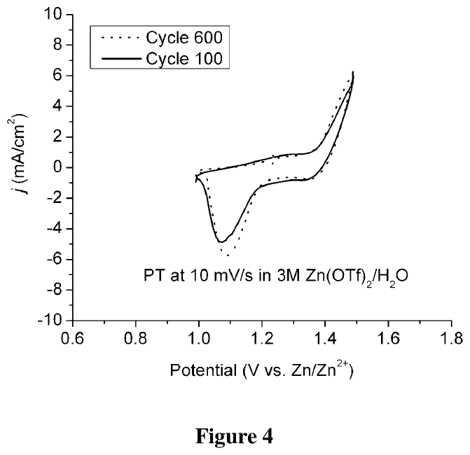

A reversible redox process was observed at 1.25V vs. Zn/Zn²⁺ electrode as shown in FIG. 4. The CV data shows that an electrochemical cell with zinc anode and PT cathode would be ˜1.25V. The CV scans were running up to 600 cycles. To our surprise, we discovered the remarkable stability of PT and it's complex PT⁺OTf⁻ upon oxidation and reduction in Zn(OTf)₂/H₂O electrolyte.

As presented in FIG. 4, the current at 600 cycle was slightly higher than the current at the cycle 100. The increased current is due to stability of the PT+OTf⁻ complex and slow wettability of the electrode.

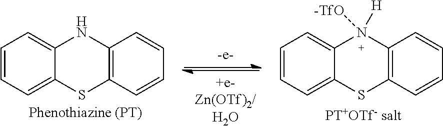

### Example 5: Zinc/Phenothiazine (PT) Electrochemical Cell in 3M Zn(OTf)2/H2O Electrolyte

This example describes a zinc/phenothiazine (PT) electrochemical cell in 3M Zn(OTf)₂/H₂O electrolyte. After observing the remarkably stable CV cycling data for PT molecule (Example 4, FIG. 4), we planned to assemble a zinc/PT cell in 3M Zn(OTf)₂/H₂O electrolyte. The anode for this example is comprised of a mixture of zinc powder (99.9%, EverZinc Group SA, Belgium), super P C65 (IMERYS Graphite and Carbon) and PTFE with the wt % of 60:30:10. The 60 wt % of zinc and 30 wt % of conducting carbon (SPC65, Timcal) were mixed using a mortar and a pestle, and then added H₂O: EtOH (1:1 by vol) and then 10 wt % of PTFE (60% solution in H₂O, FLUOROGISTX).

The overall mixture was spin-mixed and centrifuged at 2000 rpm for 1 minute. The mixture was dried at 80° C. for about 6 hours to remove H₂O and EtOH, completely. The solid mixture was then rolled using a hand roller into a free-standing film. A 1 cm² free-standing film was punched out as an anode. The cathode film was prepared by following the same procedure as mentioned above except using phenothiazine (PT, 98%, Sigma) as cathode active material and super P carbon (IMERYS Graphite and Carbon) as conducting additive and PTFE (60% solution in H₂O, FLUOROGISTX) as a binder with the weight ratio of 43:43:16, respectively.

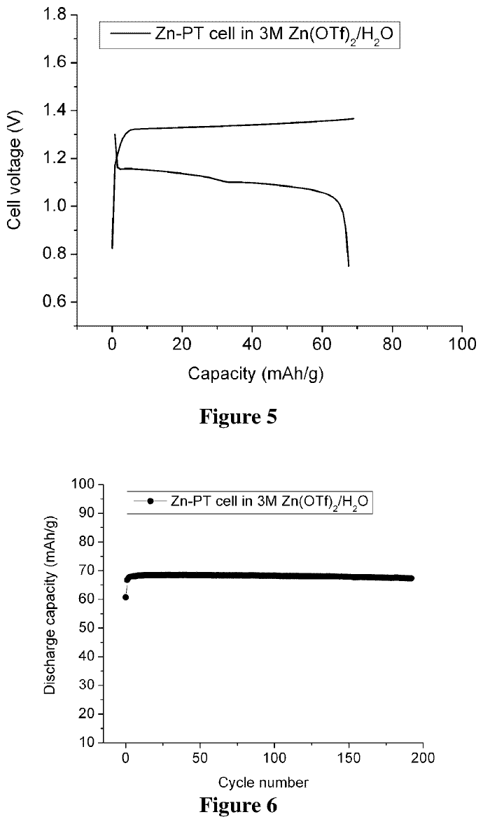

A hermetically sealed coin-type cell was assembled using the above-mentioned zinc anode and PT cathode in 3M Zn (OTf)₂/H₂O electrolyte. A sulfonated polyolefin fiber was used as separator. The cell was cycled at 2 C rate. The cycling data is presented in FIGS. 5, 6 and 7. The redox active PT molecule has a theoretical capacity at 134 mAh/g for 1e− exchange. I

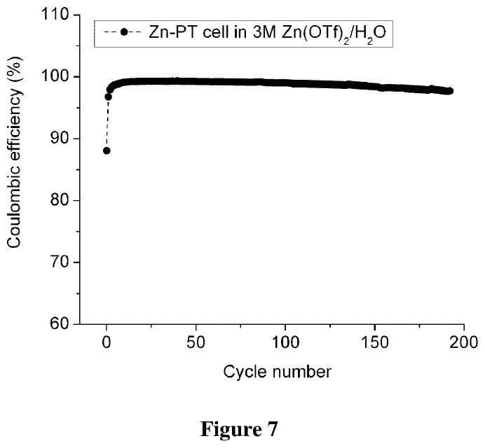

n this example, up to 50% of active material, capacity up to 67 mAh/g were utility. The voltage profile of the cell is presented in FIG. 5. As CV data showed (Example 4, FIG. 4), a 1.15V battery was obtained. The cell was cycled up to 190 cycles. No appreciable capacity loss was observed up to 190 cycles as shown in Figure 6, and a remarkable >99% coulombic efficiency was obtained, as shown in FIG. 7.

The results are unexpected since PT is a monomer molecule, which tends to dissolve in the electrolyte during cycling. This excellent cycling data is attributable in part Lewis acidic zinc salts in aqueous electrolytes. Accordingly, zinc battery with zinc carbon composite anode and a phenothiazine-based p-type organic molecule is advantageous over the other aqueous electrolyte based cells.

Phenothiazine is a very cheap molecule, synthesized from diphenylamine and elemental sulfur, which is a by-product of oil and gas. This type of cheap, environmentally friendly, safe and high-rate battery can be a good replacement of Lead acid battery for grid storage, and other stationary applications.

### Example 6: Zinc/Phenothiazine Cell in 2M Zn(OTf)2+1M LiTFSI in H2O Electrolyte

This example describes the zinc/phenothiazine cell in 2M Zn(OTf)2+1M LiTFST in H2O electrolyte. LiTFST salt was added in combination with Zn(OTf)2 in the electrolyte to reduce the water activity of the electrolyte and widen the electrochemical stability window of water. The anode and cathode were prepared the same way as described in Example 5.

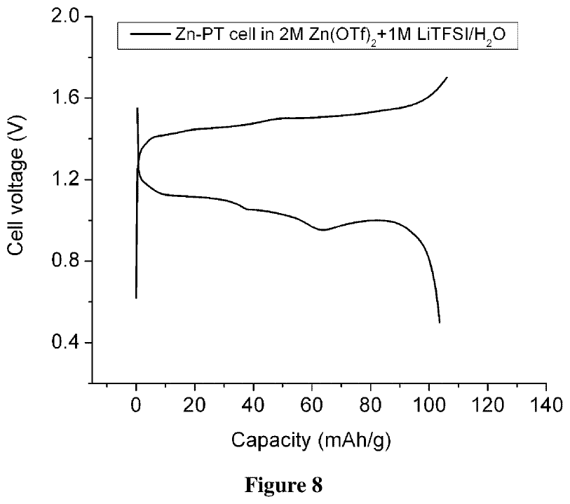

A coin-type battery was assembled, and the cell was cycled at 2 C rate with the voltage cutoff at 1.7V to 0.5V. The voltage profile is shown in FIG. 8. A 1.15V battery was obtained in this electrolyte, which is similar to the voltage profile obtained in Example 5 (FIG. 5).

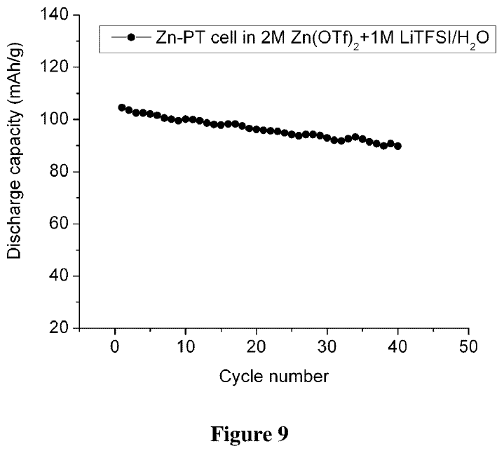

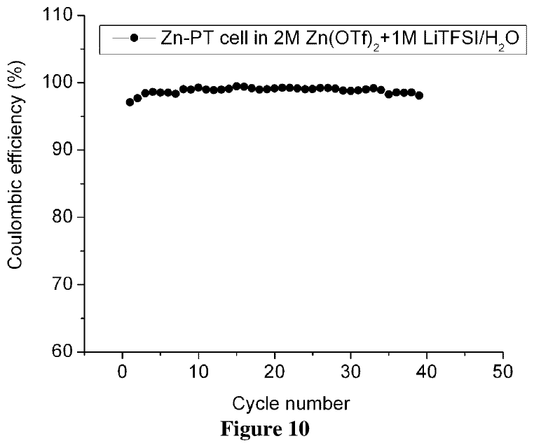

Even though the discharge voltage is similar, however, 75% of the theoretical capacity of PT was utilized. There is a slight decay of discharge capacity during cycling, as shown in FIG. 9. LiTFSI salt was used in water-in-salt electrolyte, known to have widen the voltage window of water, and known to suppress HER and OER. Although coulombic efficiency was >98%, as shown in FIG. 10, but slight capacity decay could be due to the presence of TFSI anion. PT+TFSI-complex can have some solubility in the aqueous electrolyte.

### Example 7: Zinc/Phenothiazine Cell in 23 wt % of Zn(ClO4)2, 16 wt % of LiTFSI and 7 wt % of NaClO4 in H2O Electrolyte

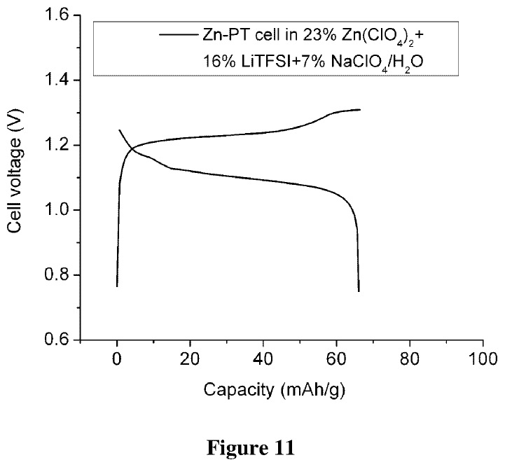

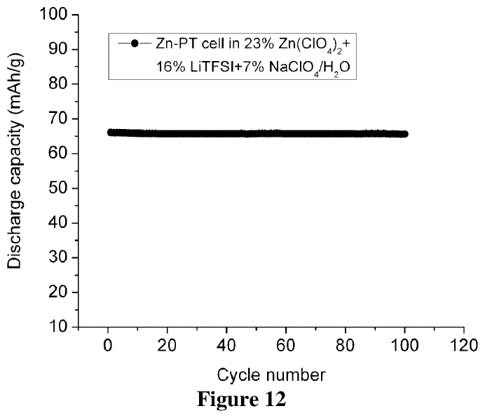

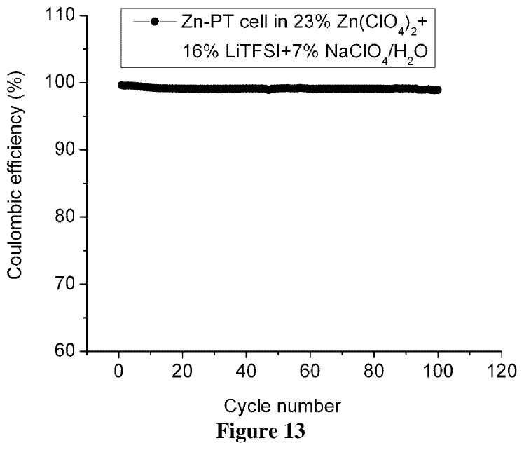

In this example, the anode and cathode were prepared by following the same procedure as described in Examples 5 and 6. Since Zn(OTf)2 is an expensive salt, it can be replaced by a cheap zinc salt such as Zn(ClO4)2. A hybrid mixture of 23 wt % of Zn(ClO4)2, 16 wt % of LiTFSI and 7 wt % of NaClO4 in H2O was used as electrolyte. The zinc-carbon/PT cell was cycled at 2 C rate. The voltage profile is presented in FIG. 11. A 1.15V battery was obtained with 50% utilization of the PT cathode material. No appreciable capacity degradation was observed up to 100 cycles, as shown in FIG. 12. The coulombic efficiency was found to be >99%, as shown in FIG. 13. The data is similar to the data obtained in Example 5.

NaClO4 was used since it has very high solubility in water (209.6 g/100 mL at 25° C.), and it is a very cheap salt. It could be a good additive salt to suppress OER at cathode and HER at zinc anode for this type of aqueous energy systems.

### Example 8: Zinc/PT2S Coin-Type Cell and its Properties

In this example, the zinc-carbon composite anode was prepared the say as described in Examples 5, 6. A dimer of phenothiazine linked via thioether (—S—) linkage (PT2S) was used as cathode active material. The dimer PT2S is synthesized in our laboratory. The synthetic procedure and its characterization are described in the synthetic section.

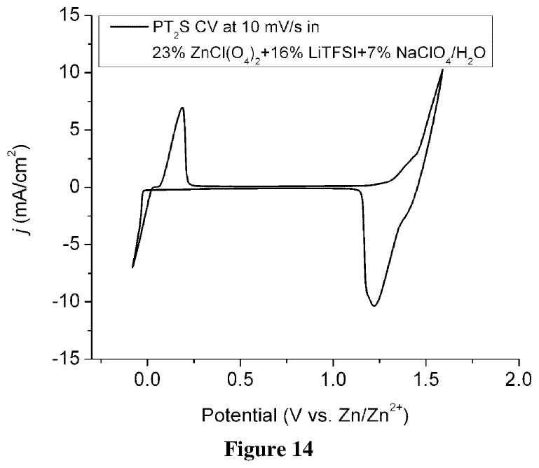

The cathode was prepared by mixing 55 wt % of PT2S of dimer, 30 wt % of Ketjan black (Timcal) and 15 wt % of PTFE (60% in H2O) in H2O:EtOH (1:1 by vol). The mixture was mixed by a Thinky centrifuged at 2000 rpm for 1 min. A free-standing electrode was prepared from the dried mixture by using a hand-roller. The cyclic voltammetry (CV) was performed in a hybrid mixture of 23% of Zn(ClO4)2, 16 wt % of LiTFSI and 7 wt % of NaClO4 in H2O electrolyte. A CV data in presented in FIG. 14.

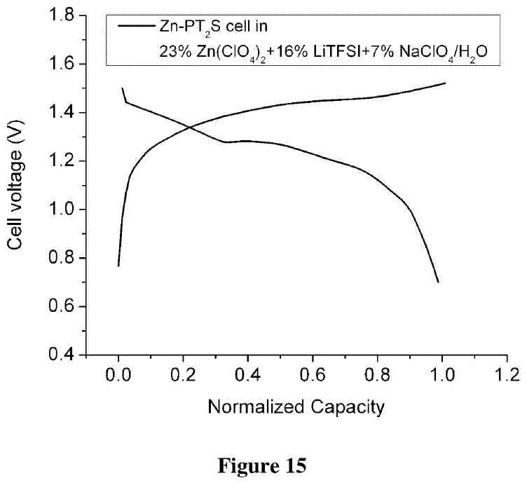

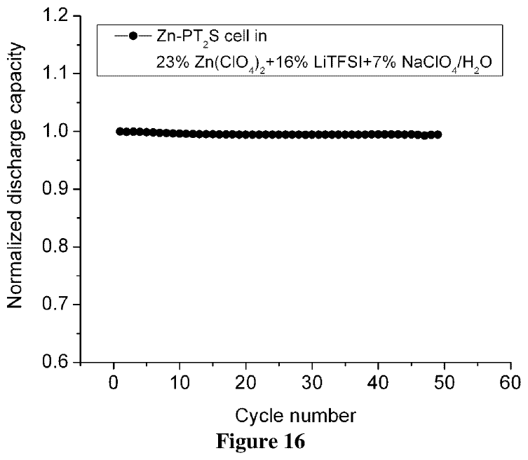

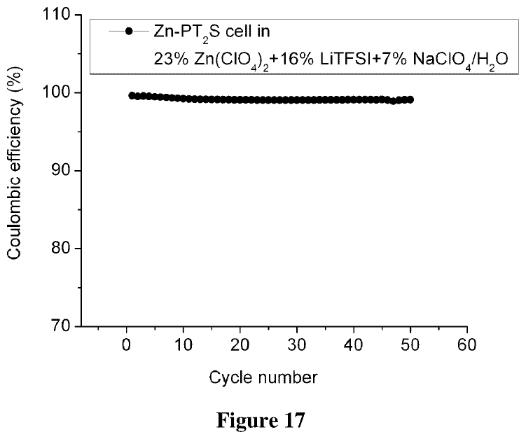

A reversible redox peak at 1.35V vs. Zn/Zn2+ was obtained, which is sharper redox peak than the CV of PT molecule (FIG. 4) indicating the faster electron kinetics in PT2S dimer electrode. Then a coin-type cell was assembled using a sulfonated polyolefin fiber as separator and a hybrid mixture of 23% of Zn(ClO4)2, 16 wt % of LiTFSI and 7 wt % of NaClO4 in H2O as electrolyte. The cell was cycled at 2 C rate up to 50 cycles. The voltage profile is presented in FIG. 15, which shows a 1.15V zinc/PT2S battery. The voltage is similar to the voltage profile obtained in Example 6, but 75% utilization of the active material is used compared to 50% in Example 4. No appreciable capacity decay was obtained up to 50 cycles, as presented in FIG. 16. The coulombic efficiency is found to be >99% (FIG. 17), which is similar to the cell presented in Examples 4, 6.

### Example 9: Zinc/PMPTS Coin-Type Cell and its Properties

In this example, a poly└10-methylphenothiazine┘ sulfide (PMPTS) polymer was used as cathode active material. The synthesis of PMPTS and its characterization are presented in synthetic section. The cathode was prepared as follows: to a well-mixed mixture of 43 wt % PMPTS, 43 wt % of super P carbon added 14 wt % of PTFE and thereafter added H2O: EtOH (1:1 by vol), and spin-mixed, centrifuged at 2000 rpm for 1 minute.

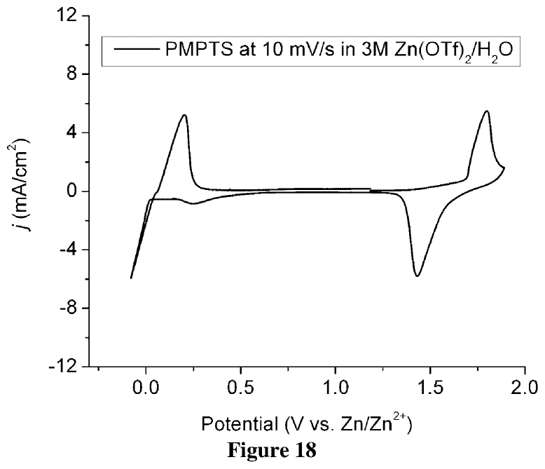

The dried mixture was rolled into a free-standing electrode. The cyclic voltammetry (CV) experiment was performed in 3M Zn(OTf)2/H2O using the three electrode described in the previous examples. The cyclic voltammogram at 10 mV/s is presented in FIG. 18. A reversible redox peak at 1.45V vs. Zn/Zn2+ was obtained, which is ˜300 mV higher than the redox peak found in PT molecule in the same electrolyte (FIG. 4).

The redox process at higher voltage could be due to the presence of electron donating N-methyl group in the polymer moiety. The zinc-carbon composite anode was prepared the same as described in the previous examples. A coin-type electrochemical battery was fabricated by using zinc-carbon composite anode and PMPTS cathode. A sulfonated polyolefin fiber as separator and 3M Zn(OTf)2/H2O as electrolyte.

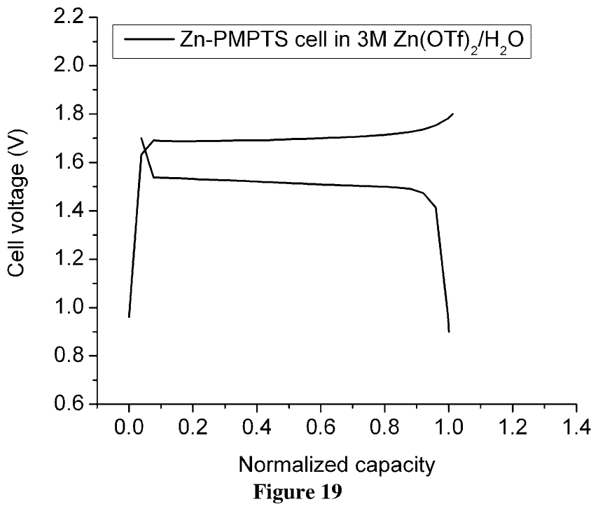

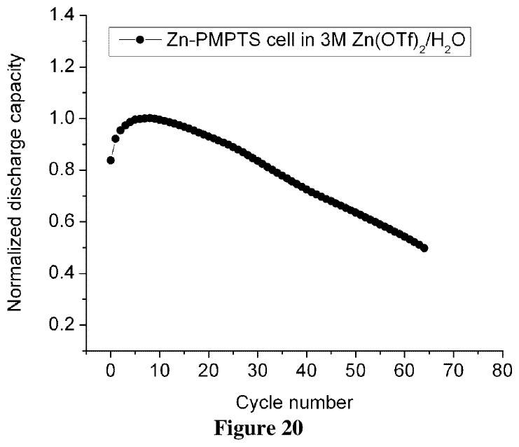

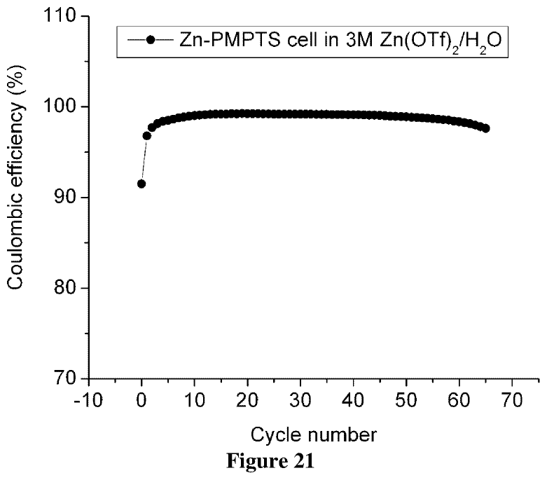

The cell was cycled at 2 C rate using voltage the cutoffs of 1.75V-0.8V. The voltage profile of the battery is presented in FIG. 19. A 1.45V battery was obtained, as expected from CV data (FIG. 18), which is ˜300 mV higher than the voltage obtained from the cell in Example 4 with the same electrolyte. To our surprise, a rapid capacity decay was obtained (FIG. 20), that could be due to the higher utilization (80%) of the active material during cycling. A skilled person based on the present disclosure can make cells in different electrolyte with high salt concentrations. FIG. 21 shows a change of coulombic efficiency of zinc/PMPTS polymer electrochemical cell using 3M Zn(OTf)₂ aqueous electrolyte during a charging and discharging cycle.

### Example 10: Zinc/PMPTS Coin-Type Cell with 23 wt % of Zn(ClO4)2, 16 wt % of LiTFSI, 7 wt % of NaClO4 Aqueous Electrolyte

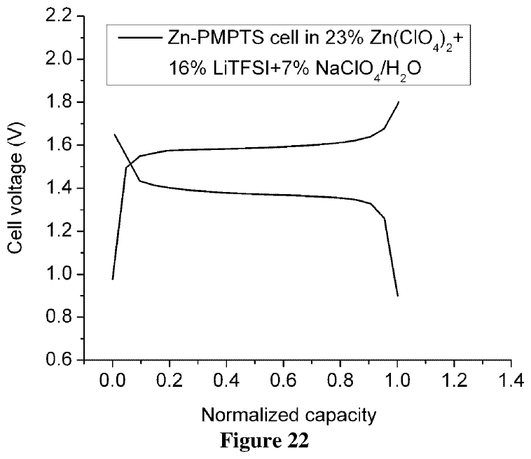

In this example, same anode and cathode were used as in example 9 except the a hybrid mixture of 23 wt % of Zn(ClO4)2, 16 wt % of LiTFSI, 7 wt % of NaClO4 is used a electrolyte. A coin cell was fabricated and cycled at 2 C rate with the voltage cutoffs of 1.75V to 0.8V. The voltage profile is presented in FIG. 22. A 1.40V battery is obtained, which is 50 mV lower than the cell voltage obtained in 3M Zn(OTf)2 electrolyte.

The lower voltage could be to be due to the lower ionic conductivity in highly concentrated electrolyte of 23% Zn(ClO4)2+16 wt % LiTFSI+7 wt % NaClO4 in H2O. The cell was cycled up to 200 cycles. Slight capacity decay was observed, but much better capacity retention than the cell described in Example 9 with 3M Zn(OTf)2 electrolyte. A >99% coulombic efficiency was obtained, which is also improvement from the Example 9. In this example, 55% capacity of the PMPTS theoretical capacity is utilized.

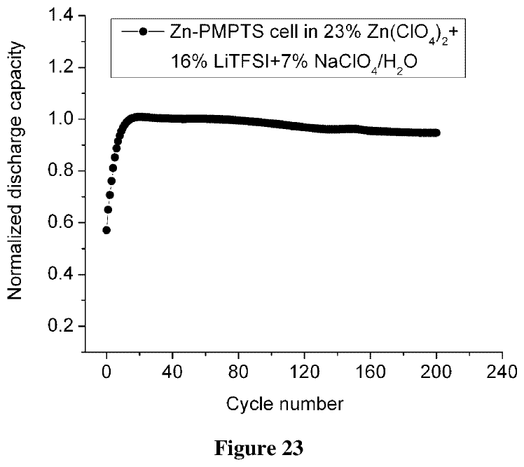

FIG. 23 shows a change of discharge capacity of a zinc/PMPTS electrochemical cell using 23 wt % of Zn(ClO₄)₂, 16 wt % of LiTFSI, 7 wt % of NaClO₄ in H₂O aqueous electrolyte over a plurality of charging and discharging cycles at 5 C rate.

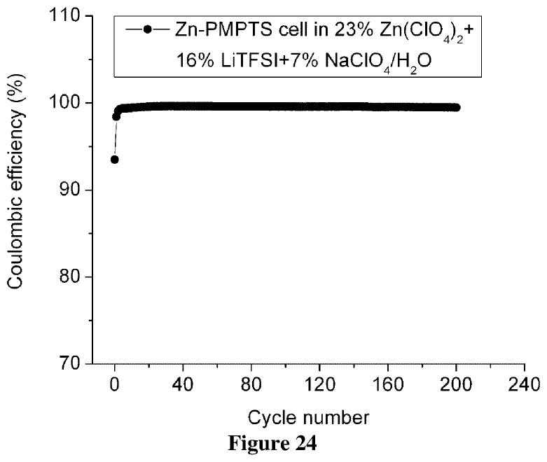

FIG. 24 shows a change of Coulombic efficiency of a zinc/PMPTS electrochemical cell using 23 wt % of Zn(ClO₄)₂, 16 wt % of LiTFSI, 7 wt % of NaClO₄ in H₂O aqueous electrolyte over a plurality of charging and discharging cycles at 5 C rate.

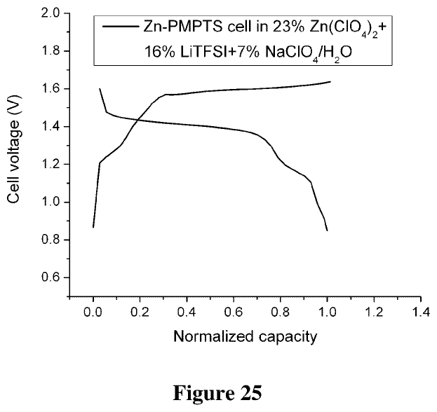

FIG. 25 shows the voltage profile of a zinc/PMPTS electrochemical cell using 23 wt % of Zn(ClO₄)₂, 16 wt % of LiTFSI, 7 wt % of NaClO₄ in H₂O aqueous electrolyte during a charging and discharging cycle at 2 C rate.

### Example 11: Zinc/PMPTS Coin-Type Cell with Optimal Electrolyte

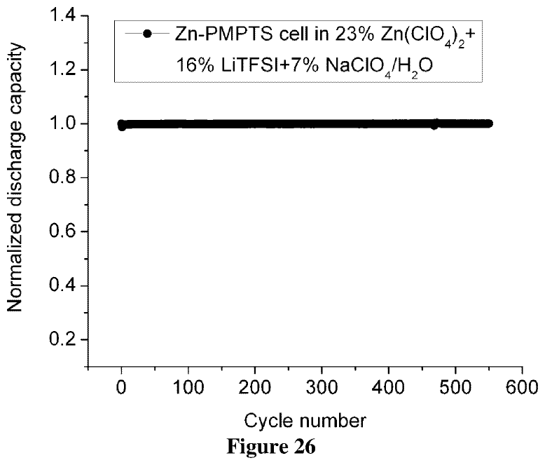

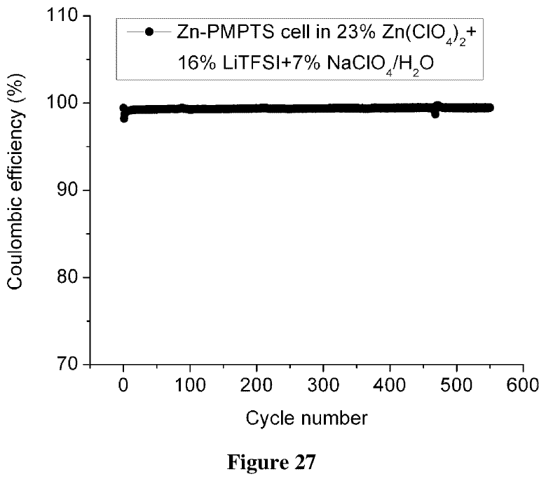

This example is same as Example 10. The cell was cycled 2.6 C rate up to 567 cycles without any capacity decay (FIG. 26). The coulombic efficiency was found to be >99% (FIG. 27). A hybrid mixture of 23% Zn(ClO4)2+16 wt % LiTFSI+7 wt % NaClO4 in H2O is found to be the best electrolyte for PMPTS cathode material.

### Example 12: Zinc/PT Cell and Unexpected Results

In this example, PT molecule is used as cathode active material in 30 wt % ZnBr2/H2O electrolyte. The composition of the active material (PT) and conducting carbon (Super P) and binder (PTFE) for the cathode was 70:20:10 wt %. The 70 wt % PT and 20 wt % SP were well-mixed using a mortar and pestle. A 50 wt % of H₂O:EtOH (1:1 vol) was added into the mixture, and then added 10 wt % of PTFE (60% solution in H₂O).

The overall mixture was mixed with Thinky at 2000 rpm for 1 minute. The mixture was dried at 80° C. for overnight to remove H₂O and EtOH, completely. The mixture was then rolled using a hand roller into a free-standing cathode electrode. The free-standing electrode was dried at 80° C. for overnight and used in all cyclic voltammetry (CV) experiment and zinc/PT cell construction.

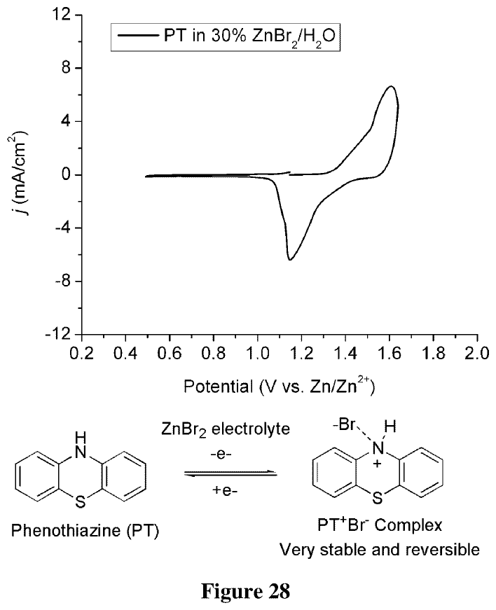

The CV experiments with PT:SP:PTFE (70:20:10) electrode was done in a three-electrode beaker cell with a Ag/AgCl (KCl std.) as reference electrode, a Pt wire as counter electrode and the above-mentioned PT (0.25 cm²) electrode as the working electrode. A Biologic SP-150 Potentiostat was used to record the electrochemical data. The CV data for PT molecule at 10 mV/s scan rate in 30% ZnBr₂ solution is presented in FIG. 28. A reversible redox process at 1.25V vs. Zn/Zn2+ was observed, as shown in the FIG. 28.

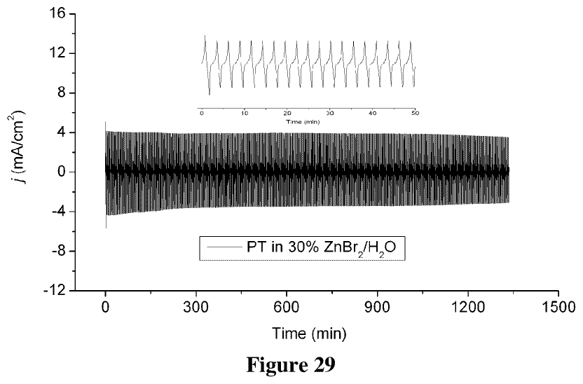

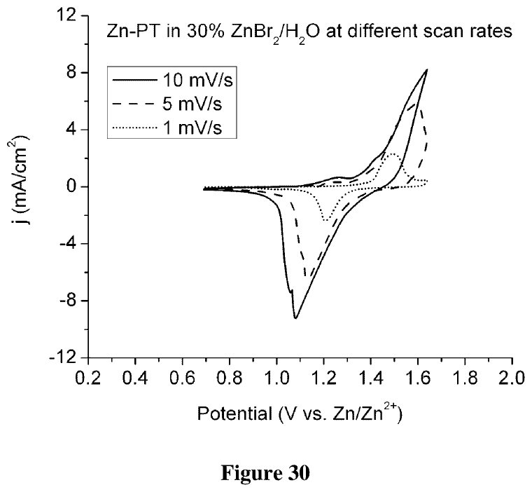

Unexpectedly, it was observed that there is no decay of current during cycling up to 500 cycles, as shown in FIG. 29 (as a current vs time plot). This data confirms that PT⁺Br⁻ complex is stable in 30 wt % ZnBr2/H2O electrolyte. The CV data in different scan rates (10 mV/s, 5 mV/s, 1 mV/s) is presented in FIG. 30, which again shows the redox current is proportional to the scan rates, which further confirms the reversible nature of PT redox process in this electrolyte.

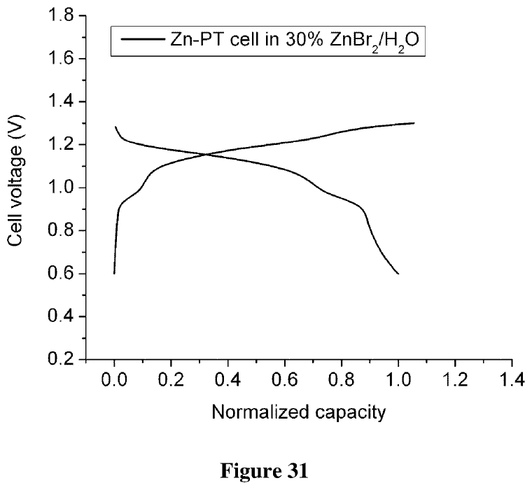

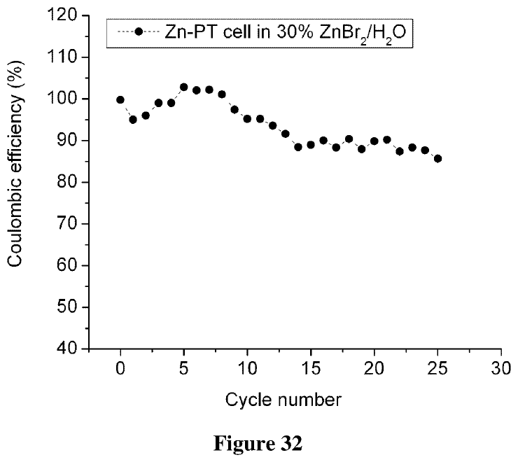

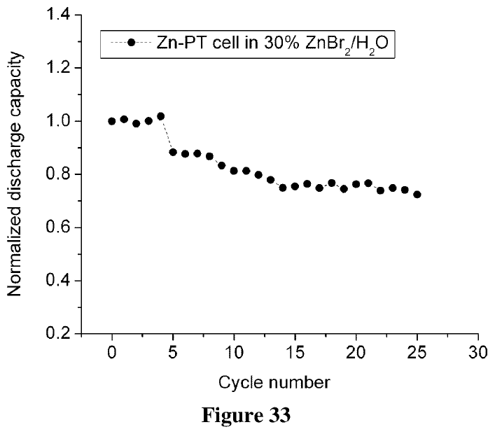

Then a zinc/PT beaker cell was constructed using zinc foil anode and PT cathode in 30 wt % ZnBr2 electrolyte. The cell was cycled at 1C up to 25 cycles. A 1.15V zinc/PT battery was obtained. The voltage profile is presented in FIG. 31. About 75% utilization of the theoretical capacity of PT molecule was obtained. The normalized discharge capacity vs cycle number is presented in FIG. 33. There is a slight decay of capacity with the cycling up to 25 cycles, which could be to the high utilization of active material. There is slight decay in coulombic efficiency as well, as shown in FIG. 32.

### Example 13: Zinc/CPT Beaker Cell and its Electrochemical Properties

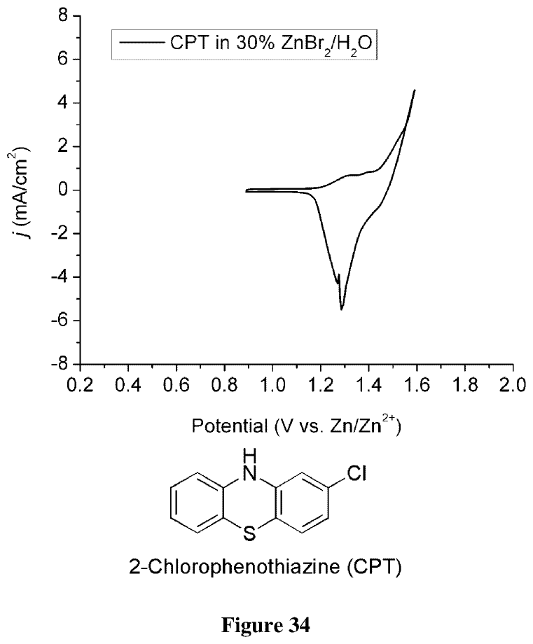

In this example, CPT molecule is used as cathode active material. The CPT cathode was constructed the same way as described in Example 12. The CV data is recorded the same way as described in Example 12. The CPT voltammogram in 30 wt % ZnBr2 electrolyte is presented in FIG. 34. A reversible redox process at 1.30V vs. Zn/Zn2+ was observed, as shown in FIG. 34.

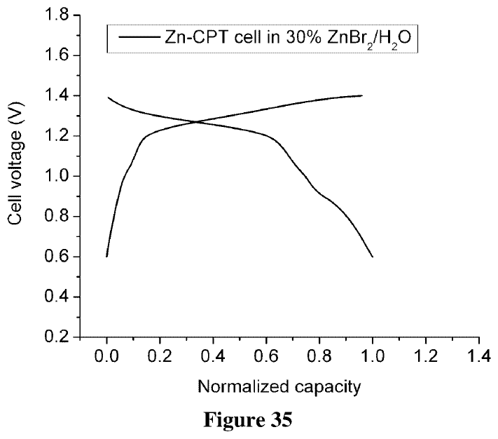

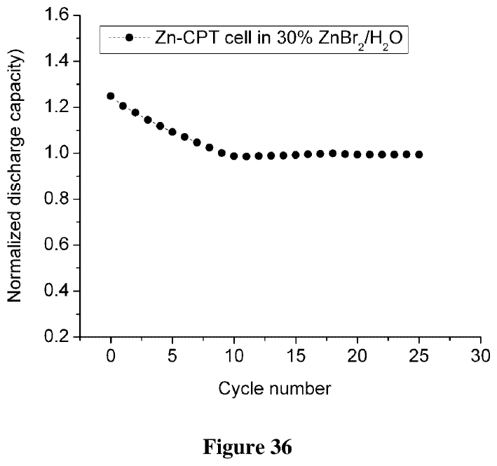

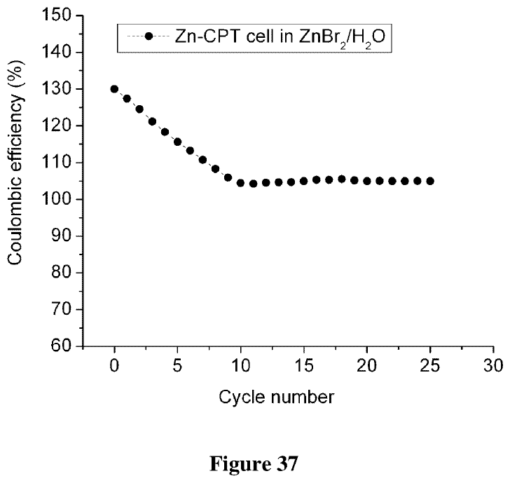

A zinc/CPT beaker cell was constructed using zinc foil as anode and CPT as cathode in 30 wt % ZnBr2/H2O electrolyte. The cell was cycled at 1 C rate. The CPT molecule has theoretical capacity of 115 mAh/g for 1e− process. The cell was cycled up to 87% of its theoretical capacity. The voltage profile shows a 1.20V battery, which is 50 mV higher than the cell with PT molecule. The data is presented in FIG. 35. The initial discharge capacity of CPT in 30 wt % ZnBr2/H2O electrolyte is found to be higher than its theoretical capacity, which could be due to presence of large excess of electrolyte in the beaker cell and redox shuttling the CPT+Br− to the anode during charge. The dissolution of active material could be prevented by making dimer or trimer or polymers from CPT molecule (Example 14). The change of discharge capacity with cycling is presented in FIG. 36. The cycling profile was stabilized after 10 cycles so as the coulombic efficiency (FIG. 37).

### Example 14: Zinc/PT2S Beaker Cell and Electrochemical Properties

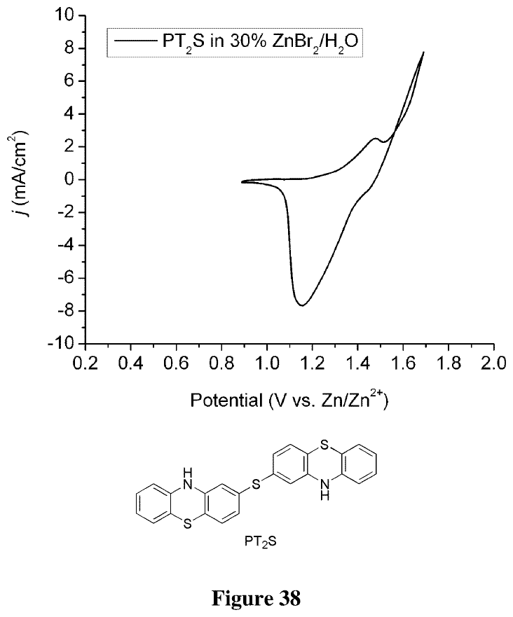

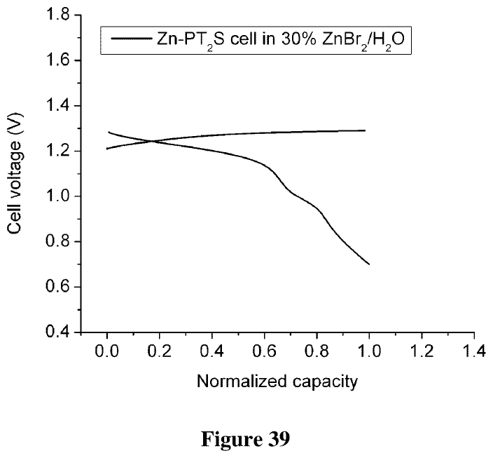

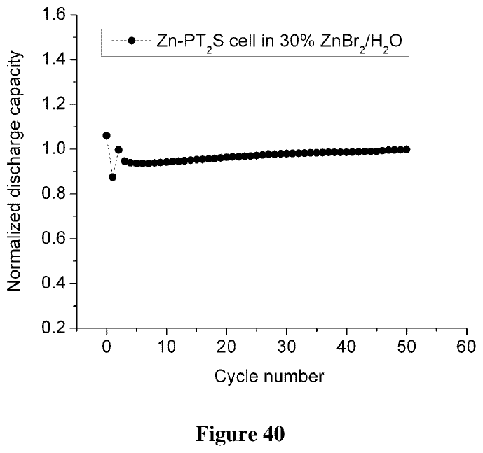

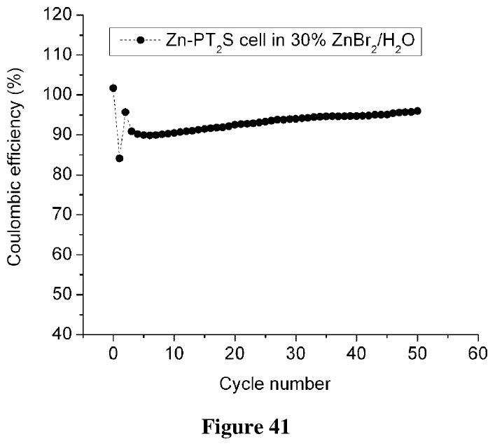

In this example, PT2S dimer is used as a cathode material. The idea to use dimer as cathode active material is to prevent dissolution of active material during cycling. The synthesis of PT2S dimer and its characterization by NMR is described in the synthetic section of this filling. The PT2S cathode is formulated the same way as described in Example 13. The CV data in 30 wt % ZnBr2/H2O electrolyte is recorded using the same beaker cell described in Example 13. The CV data is presented in FIG. 38. A reversible redox peak at 1.30V vs Zn/Zn2+ was observed, which is the same as the CPT molecule (FIG. 34). A zinc/PT2S electrochemical beaker cell is constructed in 30 wt % ZnBr2/H2O electrolyte. The cell was cycled at 1 C rate and cycled up to 25 cycles. The voltage profile, change of discharge capacity vs. cycle number, and the change of coulombic efficiency vs. cycle number are presented in FIGS. 39, 40, 41, respectively. A 1.20V battery was obtained (FIG. 39), which is similar to CPT molecule (FIG. 35). The PT2S dimer has theoretical capacity of 125 mAh/g for 1e− exchange. About 88% of the theoretical capacity was obtained. The coulombic efficiency was found to be 90% during the initial cycling. The low coulombic efficiency could be due to the high utilization of the active material, and some dissolution of active material due to the large excess of electrolyte in the beaker cell. A skilled person based on the present disclosure can synthesize trimer and polymer to mitigate the dissolution issue.

### Example 15: Zinc/PT2MPT Electrochemical Beaker Cell in 30 wt % ZnBr2/H2O Electrolyte

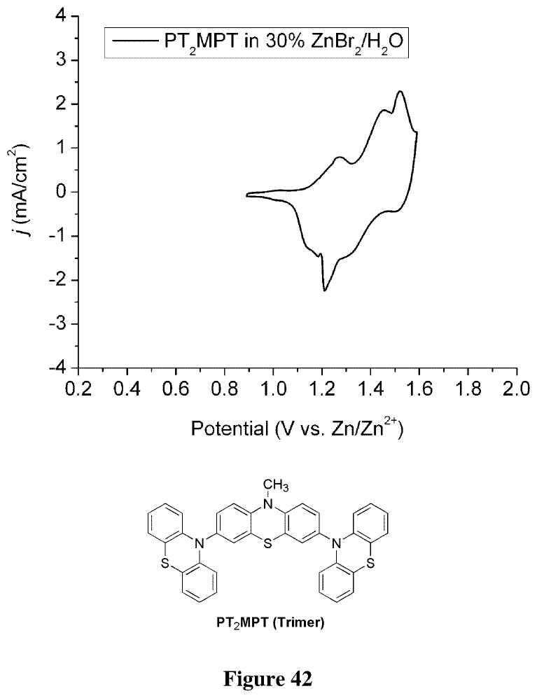

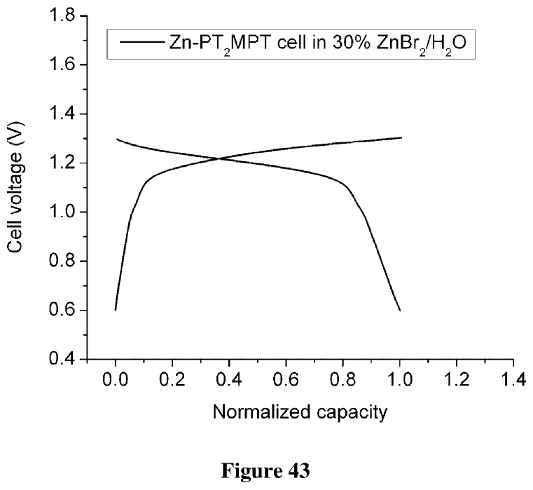

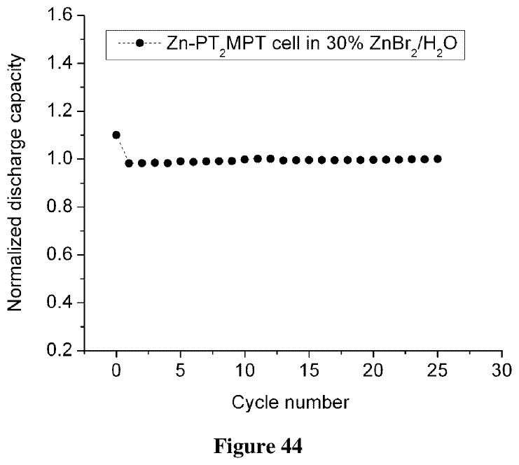

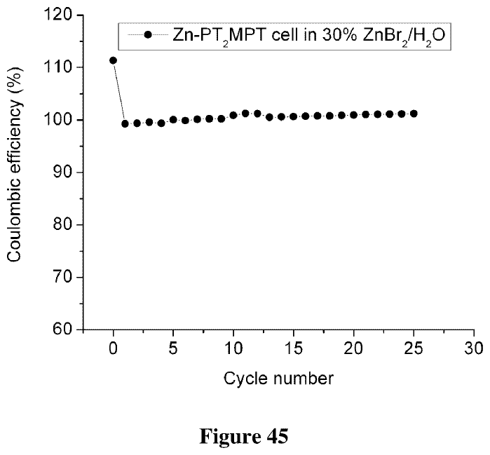

In this experiment, a trimer PT2MPT was used as cathode active material. The synthesis of PT2MPT and its characterization is described in the synthetic section of this filling. The PT2MPT cathode is formulated the same way as described in Example 13. The CV data in 30 wt % ZnBr2/H2O electrolyte is recorded using the same three electrode beaker cell as described in Example 13. The CV data is presented in FIG. 42. PT2MPT has three redox centers in its structure and three reversible redox peaks at 1.20V, 1.25V and 1.30V vs Zn/Zn2+ were observed (FIG. 42). A zinc/PT2MPT electrochemical beaker cell is constructed in 30 wt % ZnBr2/H2O electrolyte. The cell was cycled at 1 C rate and cycled up to 50 cycles. Voltage profile shows a 1.20V battery, as shown in FIG. 43, which is similar to CPT (FIG. 35) and PT2S (FIG. 39). The PT2MPT trimer has theoretical capacity of 132 mAh/g for 1e− exchange. About 75% of the theoretical capacity was obtained. The change of capacity with cycle number is presented in FIG. 44. Trimer, PT2MPT shows a stable capacity retention over 25 cycles (FIG. 44). The coulombic efficiency was found to be >99%, as shown in FIG. 45. These data show that trimer (PT2MPT) has fast redox kinetics and limited solubility in 30 wt % ZnBr2/H2O electrolyte.

As will be appreciated from the above Examples, the features and performance of these tricyclic redox active compounds, their dimers, trimers and polymeric materials herein described support their use as organic electrode materials suitable for a wide range of primary or rechargeable applications, such as stationary batteries for emergency power, local energy storage, starter or ignition, remote relay stations, communication base stations, uninterruptible power supplies (UPS), spinning reserve, peak shaving, or load leveling, or other electric grid electric storage or optimization applications. Small format or miniature battery applications including watch batteries, implanted medical device batteries, or sensing and monitoring system batteries (including gas or electric metering) are contemplated, as are other portable applications such as flashlights, toys, power tools, portable radio and television, mobile phones, camcorders, lap-top, tablet or hand-held computers, portable instruments, cordless devices, wireless peripherals, or emergency beacons. Military or extreme environment applications, including use in satellites, munitions, robots, unmanned aerial vehicles, or for military emergency power or communications are also possible.

### Example 16: Synthesis of Tricyclic Dimer PT2S

A tricyclic dimer PT2S was synthesized using the technique identifiable by a skilled person.

The synthetic scheme is presented in as follows:

PT₂S Synthetic Scheme:

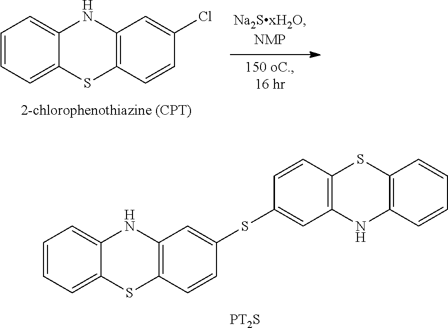

In particular as shown in the scheme and the synthetic procedure is as follows: To a solution of 2-chlorophenothiazine (97%. Sigma, 5 mmol) in NMP (99.5%, Sigma, 12 mL) under argon atmosphere, added solid Na2S.xH2O (60%, Sigma. 2.5 mmol) under argon. The mixture was stirred at room temperature for 10 min and then started heating at 150° C. After heating for 16 hours, the solution was cooled down to room temperature, and added H2O (5 mL) and 5 mL of 10% HCl in H2O. The mixture was stirred for 10 min. The off-white precipitate was filtered off and washed with copious amount of water. The dimer product (PT2S, 90% yield) was dried at 120° C. for overnight under vacuum. The same product (PT2S) was also synthesized by above-mentioned procedure in sulfolane and sulfolane: NMP (1:1) as reaction medium, respectively. The product PT₂S was characterized by ¹H-NMR (400 MHz) and Cyclic Voltammetry (CV) data analysis. The CV data shows one redox peak for only one compound (FIG. 38), no other peaks for impurities were observed. The NMR data are as follows: ¹H-NMR (400 MHz, DMSO-d6) δ 8.75 (2H, s, NH), 6.89 (4H, d, J=8.40 Hz), 6.75 (s, 2H), 6.98-6.80 (m, 4H), 6.73 (4H, dd, J=8.2, 3.1 Hz). The PT2S is a symmetric dimer molecule, so the chemical shifts for ¹H-NMR is found to be symmetrical. The chemical shift for two NH protons was observed at δ 8.75 ppm, and the integration was found to be 2. The two protons at position C-2 and C-2′ are also appeared at δ 6.75 ppm as singlet. The doublet at δ 6.89 (d, J=8.40 Hz) for the protons at C-4 and C-4′ positions. Since the molecule is symmetric, both protons appeared at the position as doublet with ortho-coupling constant of 8.40 Hz. Overall, the NMR data analysis confirmed the above structure, and also confirmed that there is no other impurities in the molecule.

### Example 17: Synthesis of PMPTS

This is a two-step synthesis, and was synthesized as follows:

Step 1: To a solution of 10-methylphenothiazine (MPT, 98%, Sigma) (15 g, 70.3 mmol) in acetonitrile (>99%, Sigma, 275 mL) was added NBS (99%, Sigma, 26.91 g, 151.2 mmol) in several portions at room temperature over 20 min. The resulting mixture was allowed to stir at room temperature for 11 hours. A solid was precipitated out. The precipitate was filtered, washed with cold acetonitrile. The filtrate was recovered, filtered through a plug of silica gel and then solvent reduced to ⅓ the initial volume using a rotary evaporator. The precipitated was filtered again, washed with cold acetonitrile, and then combined solid was dried under high vacuum at 65° C. A total of 21 g of 3,6-dibromo-10-methylphenothiazine (90% yield) was obtained. The compound was characterized by NMR data analysis. ¹H-NMR (300 MHz, CDCl₃) δ 3.03 (3H, s, N—CH₃), 7.23 (2H, s), 7.26 (2H, dd, J=8.4, 2.1 Hz), 6.62 (2H, d, J=8.7 Hz).

Step 2: The PMPTS polymer was synthesized as follows: To a solution of 3,6-dibromo-10-methylphenothiazine (4 mmol) from step 1 in sulfolane (99%, Sigma, 10 mL) under argon atmosphere, added solid Na2S.xH2O (60%, sigma, 4 mmol) under argon. The mixture was stirred at room temperature for 10 min and then started heating at 150° C. After heating for 8 hours at 150° C., the reaction mixture was allowed cool down to room temperature. At room temperature H2O (5 mL) was added and stirred for 10 min. The precipitate was filtered off and washed with copious amount of water and acetone. The polymer product (PMPTS, 91% yield) was dried at 120° C. for overnight under high vacuum. The polymer was characterized by CV data analysis and NMR data analysis. CV data (FIG. 18) shows only one redox active compound is present within the full scale from 0.00V to 2.00V vs. Zn/Zn2+(FIG. 18). ¹H-NMR (400 MHz, CDCl₃) δ 3.27 (3H, s N—CH₃), 6.62-6.60 (br. s, 2H), 7.26-7.20 (m, 4H). All ¹H-NMR chemical shifts confirm the structure of PMPTS. Due to limited solubility of the polymer in common organic solvents, and in NMR solvents, all chemical shifts appeared to be as broad peaks.

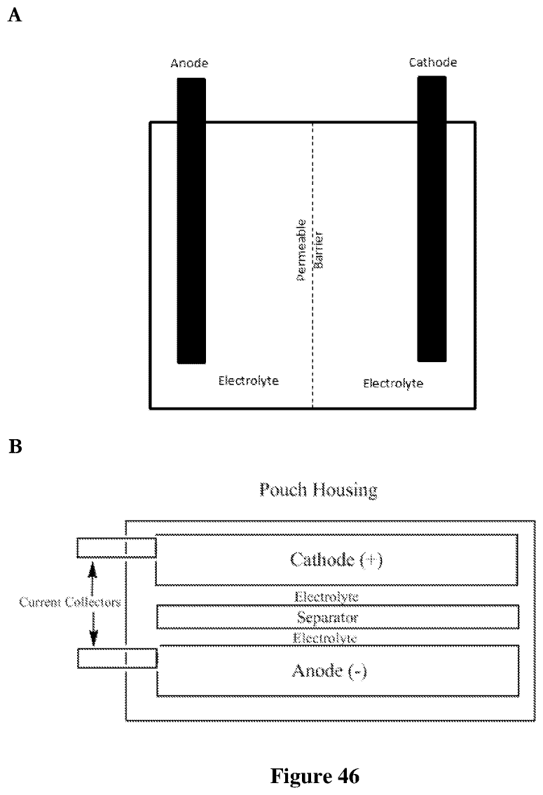

The related workflow is illustrated in FIG. 46. In particular FIG. 47 The top panel shows a schematic representation of an exemplary electrochemical cell including a Zn anode and a cathode comprising a tricyclic compound herein described. The bottom panel shows a schematic representation of an exemplary Pouch Housing electrochemical cell including a Zn anode and a cathode comprising a tricyclic compound herein described.

### Example 18: Synthesis of PMPT

Synthetic Scheme for PMPT Synthesis:

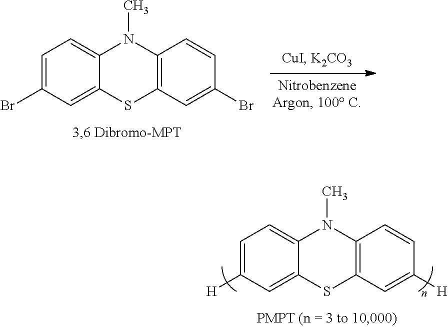

Inside the glovebox, a dry 20 mL scintillation vial equipped with a septa cap and a magnetic stir bar was added 3,6-dibromo-N-methyl phenothiazine (0.5 g, 1.35 mmol), anhydrous CuI (99.5%, Sigma, 0.05 g, 0.27 mmol), K₂CO₃ (99%, Sigma, 0.19 g, 1.35 mmol). Then added 4 mL of nitromethane to solid mixture at room temperature. The reaction vial was sealed and removed from the glovebox. The reaction mixture was heated at 100° C. for 16 hrs. The reaction mixture was cooled to room temperature and added MeOH. A brownish color precipitate was filtered-off, washed with copious amount of H₂O, MeOH and acetone. The solid was dried under vacuum at 120° C. A brownish color PMPT polymer (0.25 g, 87% yield) was obtained. The product was characterized by NMR spectroscopy. The ¹H-NMR (400 MHz, DMSO-d₆): δ 3.21 (3H, s), 7.33 (2H, s), 6.82 (1H, d, J=8.0 Hz), 6.73 (1H, d, J=8.4 Hz), 7.45 (2H, m).

### Example 19: Synthesis of PT2MPT

Synthetic scheme for PT₂MPT synthesis:

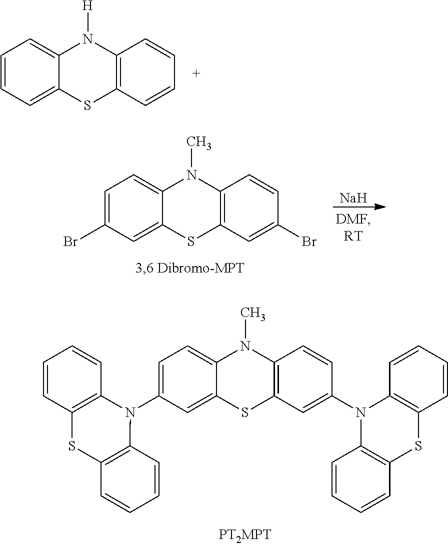

To a solution of phenothiazine (98%, Sigma, 1.13 g, 5.66 mmol) in anhydrous DMF (5 mL) was added NaH (90%, Sigma, 0.15 g, 6.06 mmol) slowly. The resulting mixture was allowed to stir at room temperature for 30 minutes. Then 3,6-diboromo-N-methyl phenothiazine (1.0 g, 2.69 mmol) was slowly added into the reaction mixture at room temperature. The reaction was stirred for 24 hours at room temperature. The work-up was done by adding H₂O (4 mL) to the reaction mixture. An off-white color material was precipitated out. The solid was collected by filtering of the liquid and washed with copious amount of water. The off-white solid of trimer, PT2MPT (1.03 g, 63% yield) was dried at 80° C. for overnight under vacuum. The compound was characterized by cyclic voltammetry (CV) experiment (Figure.) and NMR data analysis. The ¹H-NMR (400 MHz, DMSO-d₆) δ 3.33 (3H, s, N—CH₃), 8.56 (2H, s), 7.35 (4H, d, J=9.8 Hz), 6.98-6.83 (m, 8H), 6.72 (4H, t, J=7.6 Hz), 6.68 (4H, J=7.6 Hz).

### Example 20: Synthesis of a Vinyl-Phenothiazine (11c)

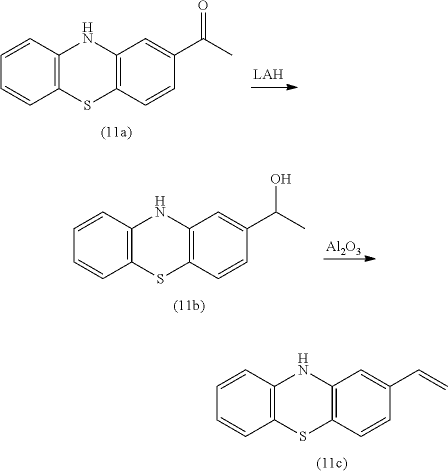

2-Acetylphenothiazine (11a) (from SigmaAldrich, catalog No. 175226) is dissolved in methylene chloride in a round bottom flask under Ar. Lithium aluminum hydride solution 1.0 M in THF (SigmaAldrich, catalog No. 16853-85-3, 1.1 eq.) is added dropwise under stirring until disappearance of the 2-Acetylphenothiazine as determined by TLC. Work up of the reaction mixture by column chromatograph produce the alcohol phenothiazine.

Alcohol phenothiazine (11b, 5 g) was dissolved in THF. To the solution was added aluminum oxide (1 g). The mixture was stirred under refluxing condition until disappearance of alcohol phenothiazine. Work up of the reaction mixture by column chromatograph produce the vinyl phenothiazine (11c).

### Example 21: Polymerization of a Vinyl-Phenothiazine (11c) to Tricyclic Compound (11d)

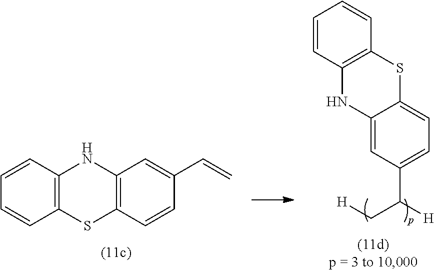

A Polymerization was carried out by the syringe technique under dry nitrogen in sealed glass tubes. A typical example for the polymerization of vinyl phenothiazine (11c) with (CH3)2C(CO2Et)I/FeCpI(CO)2/Ti(Oi-Pr)4 is given below: FeCpI(CO)2 (0.0122 g) was mixed with phenothiazine (11c) (2.75 g), dioxane (0.831 mL), and Ti(Oi-Pr)4 (0.118 mL), sequentially in this order. Immediately after adding toluene solution of (CH3)2C(CO2Et)I (0.289 mL) into the reaction mixture, the solution was placed in an oil bath at 80° C. The polymerization was terminated by cooling the reaction mixtures to −78° C. Monomer conversion was determined from the concentration of residual monomer measured by gas chromatography with tetralin as the internal standard. The quenched reaction solutions were diluted with toluene (˜20 mL) and rigorously shaken with an absorbent (Mg_{0.7}Al_{0.3}O_{1.15}, ˜5 g) to remove the metal-containing residues. After the absorbent was separated by filtration (Whatman 113V), the filtrate was washed with water and evaporated to dryness to give tricyclic compound (11d), which were subsequently dried overnight.

### Example 22: Battery Made of Zn/Tricyclic Cathode Cells

A variety of battery can be made based on the different arrangement of electrochemical cells as described herein.

FIG. 46 The top panel shows a schematic representation of an exemplary electrochemical cell including a Zn anode and a cathode comprising a tricyclic compound herein described. The bottom panel shows a schematic representation of an exemplary Pouch Housing electrochemical cell including a Zn anode and a cathode comprising a tricyclic compound herein described.

FIG. 47 shows exemplary arrangement of a plurality of electrochemical cells in a battery herein described.

FIG. 48 shows a schematic representation of an exemplary plurality of electrically connected electrochemical cells in accordance with the disclosure.

In summary, redox active polycyclic compounds and related electrode materials, electrodes, electrode chemical cells, batteries, methods and systems are herein described. In particular, tricyclic compounds having a redox potential of 0.20 V to 2.0 V with reference to Zn/Zn2+ electrode potential under standard conditions. More particularly redox active monomers, dimers, and polymers in which each monomeric unit contains a tricyclic heterocyclic structure, provide, electrode material that can be used as a cathode for an electrochemical cell further containing a zinc anode and an aqueous electrolyte. Accordingly, redox active polycyclic compounds and related electrode materials, electrodes, electrode chemical cells, batteries, methods and systems, can be used to provide in several embodiments, cheap, environmentally friendly, safe and/or high-rate battery that can be a good replacement of lead acid battery for grid storage, and other stationary applications.

The examples set forth above are provided to give those of ordinary skill in the art a complete disclosure and description of how to make and use the embodiments of the associative polymers, materials, compositions, systems and methods of the disclosure, and are not intended to limit the scope of what the inventors regard as their disclosure. All patents and publications mentioned in the specification are indicative of the levels of skill of those skilled in the art to which the disclosure pertains.

The entire disclosure of each document cited (including patents, patent applications, journal articles including related supplemental and/or supporting information sections, abstracts, laboratory manuals, books, or other disclosures) in the Background, Summary, Detailed Description, and Examples is hereby incorporated herein by reference. All references cited in this disclosure are incorporated by reference to the same extent as if each reference had been incorporated by reference in its entirety individually. However, if any inconsistency arises between a cited reference and the present disclosure, the present disclosure takes precedence.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the disclosure has been specifically disclosed by preferred embodiments, exemplary embodiments and optional features, modification and variation of the concepts herein disclosed can be resorted to by those skilled in the art, and that such modifications and variations are considered to be within the scope of this disclosure as defined by the appended claims.

It is also to be understood that the terminology used herein is for the purpose of describing particular embodiments only and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

The term “alkyl” as used herein refers to a linear, branched, or cyclic saturated hydrocarbon group typically although not necessarily containing 1 to about 15 carbon atoms, or 1 to about 6 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although again not necessarily, alkyl groups herein contain 1 to about 15 carbon atoms. The term “cycloalkyl” intends a cyclic alkyl group, typically having 4 to 8, or 5 to 7, carbon atoms. The term “substituted alkyl” refers to alkyl substituted with one or more substituent groups, and the terms “heteroatom-containing alkyl” and “heteroalkyl” refer to alkyl in which at least one carbon atom is replaced with a heteroatom. If not otherwise indicated, the terms “alkyl” and “lower alkyl” include linear, branched, cyclic, unsubstituted, substituted, and/or heteroatom-containing alkyl and lower alkyl, respectively.

The term “heteroatom-containing” as in a “heteroatom-containing alky group” refers to an alkyl group in which one or more carbon atoms is replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the term “heterocyclic” refers to a cyclic substituent that is heteroatom-containing, the terms “heteroaryl” and “heteroaromatic” respectively refer to “aryl” and “aromatic” substituents that are heteroatom-containing, and the like. It should be noted that a “heterocyclic” group or compound may or may not be aromatic, and further that “heterocycles” may be monocyclic, bicyclic, or polycyclic as described above with respect to the term “aryl.” Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, pyrimidinyl, imidazolyl, 1,2,4-triazolyl, tetrazolyl, etc., and examples of heteroatom-containing alicyclic groups are pyrrolidino, morpholino, piperazino, piperidino, etc.

The term “alkoxy” as used herein intends an alkyl group bound through a single, terminal ether linkage; that is, an “alkoxy” group may be represented as —O-alkyl where alkyl is as defined above. A “lower alkoxy” group intends an alkoxy group containing 1 to 6 carbon atoms. Analogously, “alkenyloxy” and “lower alkenyloxy” respectively refer to an alkenyl and lower alkenyl group bound through a single, terminal ether linkage, and “alkynyloxy” and “lower alkynyloxy” respectively refer to an alkynyl and lower alkynyl group bound through a single, terminal ether linkage.

The term “aryl” as used herein, and unless otherwise specified, refers to an aromatic substituent containing a single aromatic ring or multiple aromatic rings that are fused together, directly linked, or indirectly linked (such that the different aromatic rings are bound to a common group such as a methylene or ethylene moiety). Aryl groups can contain 5 to 24 carbon atoms, or aryl groups contain 5 to 14 carbon atoms. Exemplary aryl groups contain one aromatic ring or two fused or linked aromatic rings, e.g., phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituents in which at least one carbon atom is replaced with a heteroatom, as will be described in further detail infra.

The terms “cyclic”, “cyclo-”, and “ring” refer to alicyclic or aromatic groups that may or may not be substituted and/or heteroatom containing, and that may be monocyclic, bicyclic, or polycyclic. The term “alicyclic” is used in the conventional sense to refer to an aliphatic cyclic moiety, as opposed to an aromatic cyclic moiety, and may be monocyclic, bicyclic or polycyclic.

The term “isomers” as used refers to heterocyclic aromatic groups that have the same core molecular but may differ in atomic connectivity and/or location of unsaturation and is meant to include all possible structural variants. For example, as shown below, “pyrrole isomers” refers to all possible substituted variants of 1H-pyrrole and 2H-pyrrole; “indole isomers” refers to all possible substituted variants of 3H-indole, 1H-indole and 2H-isoindole, and so on:

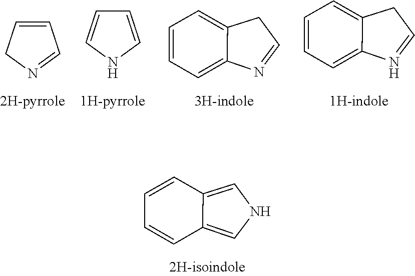

Likewise, as shown below, “triazole isomers” refers to all possible substituted variants of 1,2,4-triazole and 1,2,3-triazole; “oxadiazole isomers” refers to all possible substituted variants of 1,2,5-oxadiazole and 1,2,3-oxadiazole, and so on:

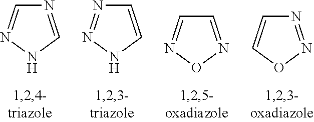

The terms “halo”, “halogen”, and “halide” are used in the conventional sense to refer to a chloro, bromo, fluoro or iodo substituent or ligand.

The term alkylene as used herein refers to an alkanediyl group which is a divalent saturated aliphatic group, with two carbon atoms as points of attachment, a linear or branched, cyclo, cyclic or acyclic structure. Exemplary alkylene includes propane-1,2-diyl group (—CH(CH3)CH2-) or propane-1,3-diyl group (—CH2CH2CH2-).

The term alkenylene refers to an alkenediyl group which is a divalent unsaturated aliphatic group, with two carbon atoms as points of attachment, a linear or branched, cyclo, cyclic or acyclic structure, at least one nonaromatic carbon-carbon double bond. Exemplary alkylene includes 2-butene-1,4-diyl group (—CH2CH═CHCH2-).

The term alkynylene refers to an alkynediyl group which is a divalent unsaturated aliphatic group, with two carbon atoms as points of attachment, a linear or branched, cyclo, cyclic or acyclic structure, at least one nonaromatic carbon-carbon triple bond. Exemplary alkylene includes 2-butyne-1,4-diyl group (—CH2C≡CCH2-).

The term “substituted” as in “substituted alkyl,” “substituted aryl,” and the like, is meant that in the, alkyl, aryl, or other moiety, at least one hydrogen atom bound to a carbon (or other) atom is replaced with one or more non-hydrogen substituents.

Examples of such substituents include, without limitation: functional groups such as halo, hydroxyl, sulfhydryl, C1-C24 alkoxy, C2-C24 alkenyloxy, C2-C24 alkynyloxy, C5-C24 aryloxy, C6-C24 aralkyloxy, C6-C24 alkaryloxy, acyl (including C2-C24 alkylcarbonyl (—CO-alkyl) and C6-C24 arylcarbonyl (—CO-aryl)), acyloxy (—O-acyl, including C2-C24 alkylcarbonyloxy (—O—CO-alkyl) and C6-C24 arylcarbonyloxy (—O—CO-aryl)), C2-C24 alkoxycarbonyl (—(CO)—O-alkyl), C6-C24 aryloxycarbonyl (—(CO)—O-aryl), halocarbonyl (—CO)—X where X is halo), C2-C24 alkylcarbonato (—O—(CO)—O-alkyl), C6-C24 arylcarbonato (—O—(CO)—O-aryl), carboxy (—COOH), carboxylato (COO—), carbamoyl (—(CO)—NH2), mono-(C1-C24 alkyl)-substituted carbamoyl (—(CO)—NH(C1-C24 alkyl)), di-(C1-C24 alkyl)-substituted carbamoyl (—(CO)—N(C1-C24 alkyl)2), mono-(C5-C24 aryl)-substituted carbamoyl (—(CO)—NH-aryl), di-(C5-C24 aryl)-substituted carbamoyl (—(CO)—N(C5-C24 aryl)2), di-N—(C1-C24 alkyl),N—(C5-C24 aryl)-substituted carbamoyl, thiocarbamoyl (—(CS)—NH2), mono-(C1-C24 alkyl)-substituted thiocarbamoyl (—(CO)—NH(C1-C24 alkyl)), di-(C1-C24 alkyl)-substituted thiocarbamoyl (—(CO)—N(C1-C24 alkyl)2), mono-(C5-C24 aryl)-substituted thiocarbamoyl (—(CO)—NH-aryl), di-(C5-C24 aryl)-substituted thiocarbamoyl (—(CO)—N(C5-C24 aryl)2), di-N—(C1-C24 alkyl),N—(C5-C24 aryl)-substituted thiocarbamoyl, carbamido (—NH—(CO)—NH2), cyano(—C—N), cyanato (—O—C≡N), thiocyanato (—S—C≡N), formyl (—(CO)—H), thioformyl ((CS)—H), amino (—NH2), mono-(C1-C24 alkyl)-substituted amino, di-(C1-C24 alkyl)-substituted amino, mono-(C5-C24 aryl)-substituted amino, di-(C5-C24 aryl)-substituted amino, C2-C24 alkylamido (—NH—(CO)-alkyl), C6-C24 arylamido (—NH—(CO)-aryl), imino (—CR═NH where R=hydrogen, C1-C24 alkyl, C5-C24 aryl, C6-C24 alkaryl, C6-C24 aralkyl, etc.), C2-C20 alkylimino (CR═N(alkyl), where R=hydrogen, C1-C24 alkyl, C5-C24 aryl, C6-C24 alkaryl, C6-C24 aralkyl, etc.), arylimino (—CR═N(aryl), where R=hydrogen, C1-C20 alkyl, C5-C24 aryl, C6-C24 alkaryl, C6-C24 aralkyl, etc.), nitro (—NO2), nitroso (—NO), sulfo (—SO2-OH), sulfonato (—SO2-O—), C1-C24 alkylsulfanyl (—S-alkyl; also termed “alkylthio”), C5-C24 arylsulfanyl (—S-aryl; also termed “arylthio”), C1-C24 alkylsulfinyl (—(SO)-alkyl), C5-C24 arylsulfinyl (—(SO)-aryl), C1-C24 alkylsulfonyl (—SO2-alkyl), C5-C24 arylsulfonyl (—SO2-aryl), boryl (—BH2), borono (—B(OH)2), boronato (—B(OR)2 where R is alkyl or other hydrocarbyl), phosphono (—P(O)(OH)2), phosphonato (—P(O)(O⁻)2), phosphinato (—P(O)(O⁻)), phospho (—PO2), phosphino (—PH2), silyl (—SiR3 wherein R is hydrogen or hydrocarbyl), and silyloxy (—O-silyl); and the hydrocarbyl moieties C1-C24 alkyl (e.g. C1-C12 alkyl and C1-C6 alkyl), C2-C24 alkenyl (e.g. C2-C12 alkenyl and C2-C6 alkenyl), C2-C24 alkynyl (e.g. C2-C12 alkynyl and C2-C6 alkynyl), C5-C24 aryl (e.g. C5-C14 aryl), C6-C24 alkaryl (e.g. C6-C16 alkaryl), and C6-C24 aralkyl (e.g. C6-C16 aralkyl).

The term “acyl” refers to substituents having the formula —(CO)-alkyl, —(CO)-aryl, or —(CO)-aralkyl, and the term “acyloxy” refers to substituents having the formula —O(CO)-alkyl, —O(CO)-aryl, or —O(CO)-aralkyl, wherein “alkyl,” “aryl, and “aralkyl” are as defined above.

The term “alkaryl” refers to an aryl group with an alkyl substituent, and the term “aralkyl” refers to an alkyl group with an aryl substituent, wherein “aryl” and “alkyl” are as defined above. In some embodiments, alkaryl and aralkyl groups contain 6 to 24 carbon atoms, and particularly alkaryl and aralkyl groups contain 6 to 16 carbon atoms. Alkaryl groups include, for example, p-methylphenyl, 2,4-dimethylphenyl, p-cyclohexylphenyl, 2,7-dimethylnaphthyl, 7-cyclooctylnaphthyl, 3-ethyl-cyclopenta-1,4-diene, and the like. Examples of aralkyl groups include, without limitation, benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like. The terms “alkaryloxy” and “aralkyloxy” refer to substituents of the formula —OR wherein R is alkaryl or aralkyl, respectively, as just defined.

The term “Periodic Table” refers to the version of IUPAC Periodic Table of the Elements dated Nov. 28, 2016, which is accessible at iupac.org/wp-content/uploads/2015/07/IUPAC_Periodic_Table-28Nov16.pdf.

When a Markush group or other grouping is used herein, all individual members of the group and all combinations and possible subcombinations of the group are intended to be individually included in the disclosure. Every combination of components or materials described or exemplified herein can be used to practice the disclosure, unless otherwise stated. One of ordinary skill in the art will appreciate that methods, device elements, and materials other than those specifically exemplified can be employed in the practice of the disclosure without resort to undue experimentation. All art-known functional equivalents, of any such methods, device elements, and materials are intended to be included in this disclosure. Whenever a range is given in the specification, for example, a temperature range, a frequency range, a time range, or a composition range, all intermediate ranges and all subranges, as well as, all individual values included in the ranges given are intended to be included in the disclosure. Any one or more individual members of a range or group disclosed herein can be excluded from a claim of this disclosure. The disclosure illustratively described herein suitably can be practiced in the absence of any element or elements, limitation or limitations, which is not specifically disclosed herein.

“Optional” or “optionally” means that the subsequently described circumstance may or may not occur, so that the description includes instances where the circumstance occurs and instances where it does not according to the guidance provided in the present disclosure. For example, the phrase “optionally substituted” means that a non-hydrogen substituent may or may not be present on a given atom, and, thus, the description includes structures wherein a non-hydrogen substituent is present and structures wherein a non-hydrogen substituent is not present. It will be appreciated that the phrase “optionally substituted” is used interchangeably with the phrase “substituted or unsubstituted.” Unless otherwise indicated, an optionally substituted group may have a substituent at each substitutable position of the group, and when more than one position in any given structure may be substituted with more than one substituent selected from a specified group, the substituent may be either the same or different at every position. Combinations of substituents envisioned can be identified in view of the desired features of the compound in view of the present disclosure, and in view of the features that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

A number of embodiments of the disclosure have been described. The specific embodiments provided herein are examples of useful embodiments of the disclosure and it will be apparent to one skilled in the art that the disclosure can be carried out using a large number of variations of the devices, device components, methods steps set forth in the present description. As will be obvious to one of skill in the art, methods and devices useful for the present methods can include a large number of optional composition and processing elements and steps.

In summary, in several embodiments, described herein are organosilicon compound, related complex that allow performance of fluorocarbon compound or olefin-based reactions and in particular polymerization of olefins to produce polyolefin polymers, and related methods and systems are described.

In particular, it will be understood that various modifications may be made without departing from the spirit and scope of the present disclosure. Accordingly, other embodiments are within the scope of the following claims.

### REFERENCES

(1) U.S. Pat. No. 6,248,474 B1.
(2) U.S. Pat. No. 10,033,039.
(4) Yanliang Liang et al., “Universal quinone electrodes for long cycle life aqueous rechargeable batteries,” Nature Materials, Vol. 16, pp. 841-848 (2017).
(5) Morita Yasushi et al., “Organic tailored batteries materials using stable open-shell molecules with degenerate frontier orbitals,” Nature Materials, Vol. 10, pp. 947-951 (2011).
(6) Zhiwei Tie et. al., “Design strategies for high-performance aqueous Zn/organic batteries,” Angewandte Chenie International Edition, Vol 59, pp. 21293-21303 (2020).
(7) Filipp A. Obrezkov et. al., “High-energy and high-power density potassium ion batteries using dihydrophenazine-based polymer as active cathode material,” The Journal of Physical Chemistry Letters, Vol 10, pp. 5440-5445 (2019).
(8) U.S. Pat. No. 9,780,412 B2.
(9) Fei Wang et. al., “Highly reversible zinc-metal anode for aqueous batteries,” Nature Materials, Vol 17, pp. 543-549 (2018)
(10) Longsheng Cao et. al. Fluorinated interphase enables reversible aqueous zinc battery chemistries,” Nature Nanotechnology, Vol 60, pp. 18845-18851 (2021).
(11) Polycyclic compound, Wikipedia, en.wikipedia.org/wiki/Polycyclic_compound.
(12) Monomer, Wikipedia en.wikipedia.org/wiki/Monomer.
(13) Yuzo Kotani, Macromolecules 2000, 33, 10, 3543-3549.

1. A tricyclic compound represented by Formula (1) 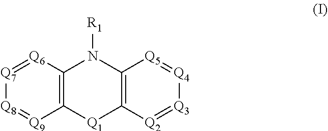
in which
Q1 is a —O—, —S— or ═NR4,
Q2 to Q9 are each independently selected from N or CR5 with the proviso that at most two of Q2 to Q5 or two of Q6 to Q9 are N,
wherein R1, and R4 are independently selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S, and
wherein the tricyclic compound has a redox potential of 0.20 V to 2.0 V with reference to Zn/Zn2+ electrode potential under standard conditions.

2. The tricyclic compound of claim 1, the tricyclic compound being represented by Formula (IA) 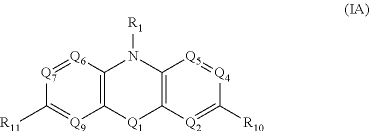
in which
Q1 is a —O—, —S— or ═NR4,
Q2, Q4 to Q7, and Q9 are each independently selected from N or CR5 with the proviso that at most two of Q2 to Q5 or two of Q6 to Q9 are N,
wherein R1, and R4 are independently selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R10 and R11 are each independently selected from H, or any one of Formula (1a) to Formula (9c),

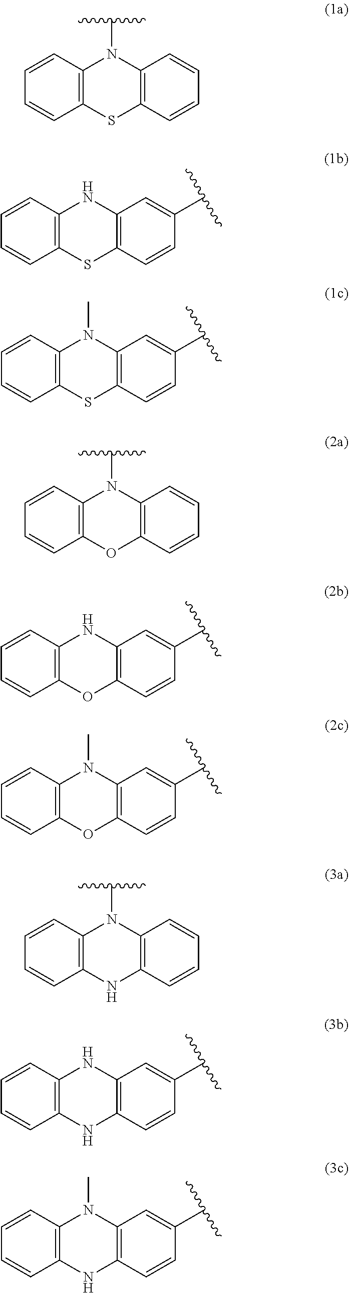

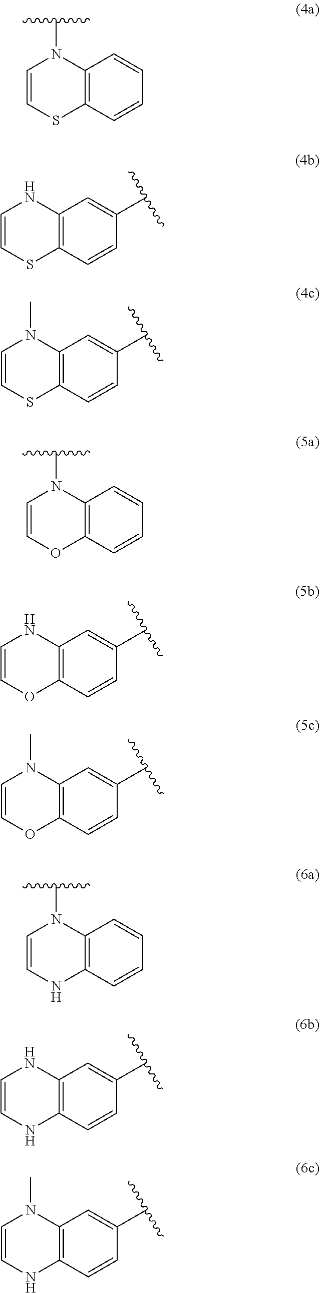

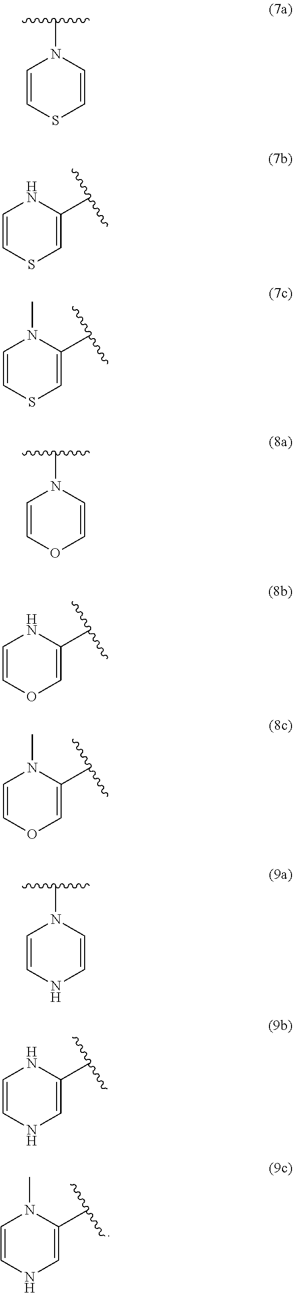

3. The tricyclic compound of claim 1, the tricyclic compound being represented by Formula (IB) 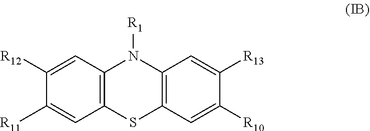
in which
wherein R1 is selected from H, or a linear or branched, C1-C4 alkyl group including methyl, ethyl, propyl, and butyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R10, R11, R12 and R13 are each independently selected from H, or any one of Formula (1a) to Formula (9c),

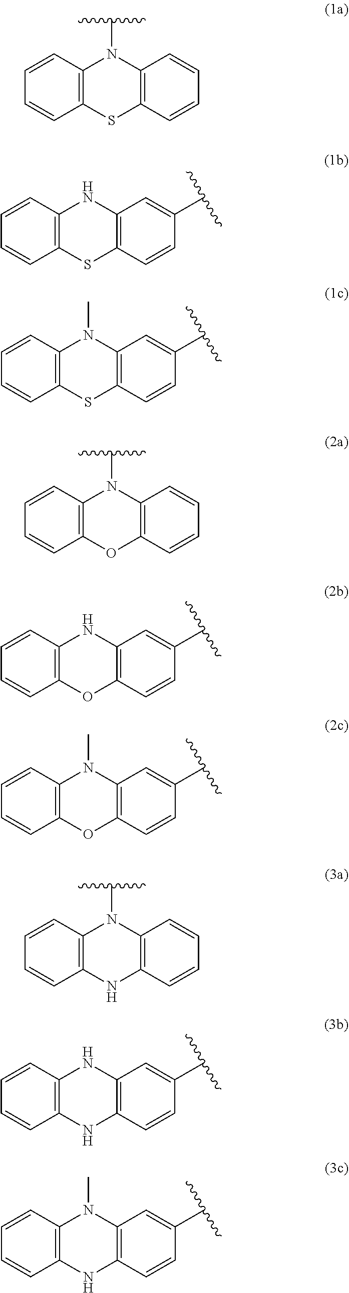

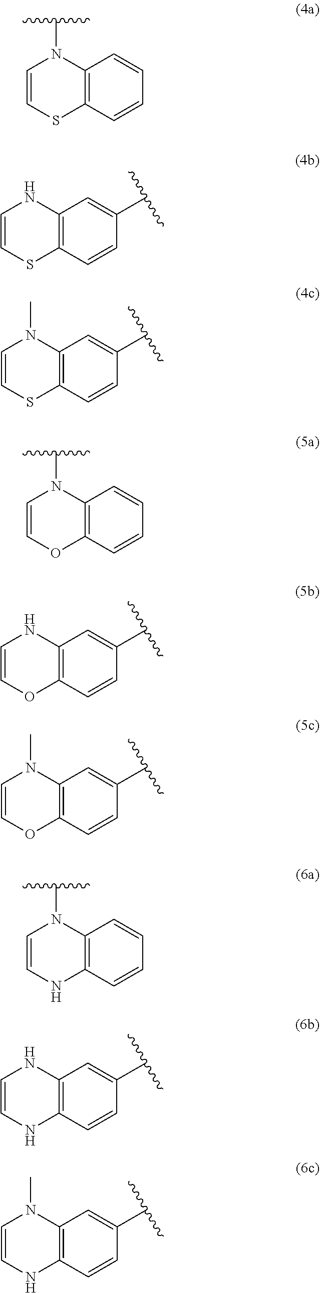

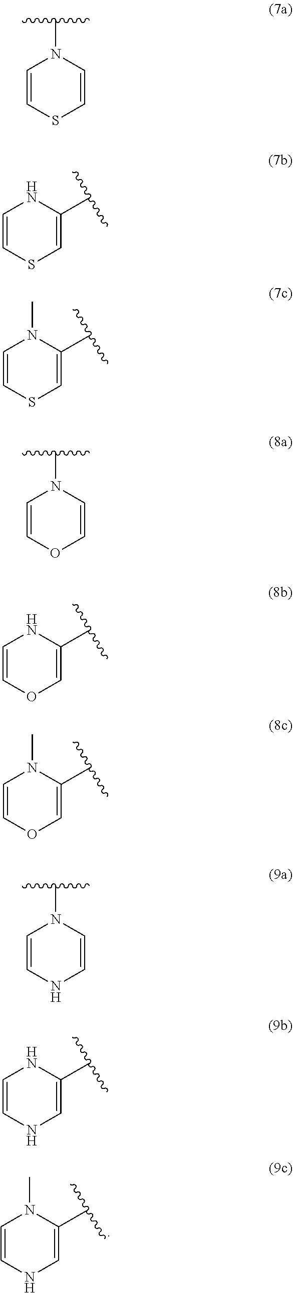

4. A tricyclic compound comprising two three-ring structures, the tricyclic compound represented by Formula (IV) 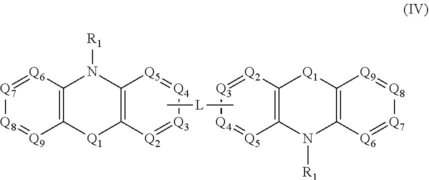
in which
Q1 is a —O—, —S— or ═NR4,
Q2 to Q5 are each independently selected from N or CR5 with the proviso that at most two of Q2 to Q5 are N and one of Q2 to Q5 is C—X wherein X is Cl, Br, or I,
Q6 to Q9 are each independently selected from N or CR5 with the proviso that at most two of Q6 to Q9 are N,
wherein R1, and R4 are independently selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S;
wherein L is null when a coupling reagent including CuI is used or O, S, NR1, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S, wherein L links between a ring carbon atom of any one of Q2 to Q5 to a ring carbon atom of any one of Q2 to Q5 of another monomeric moiety, and
wherein the tricyclic compound has a redox potential of 0.20 V to 2.0 V with reference to Zn/Zn²⁺ electrode potential under standard conditions.

5. A tricyclic compound comprising three or more three-ring structures, the tricyclic compound being represented by Formula (II) 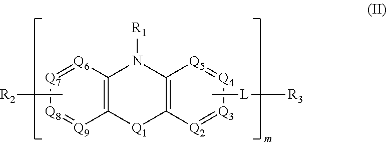
in which
Q1 is a —O—, —S— or ═NR4,
Q2 to Q9 are each independently selected from N or CR5 with the proviso that at most two of Q2 to Q5 or two of Q6 to Q9 are N,
wherein R1, and R4 are independently selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
L is null or O, S, NR1, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S, wherein L links between a ring carbon atom of any one of Q2 to Q5 to a ring carbon atom of any one of Q6 to Q9 of an adjacent monomeric moiety,
R2 and R3 are null or H,
m ranges from 3 to 10,000, and
wherein the tricyclic compound has a redox potential of 0.20 V to 2.0 V with reference to Zn/Zn2+ electrode potential under standard conditions.

6. The tricyclic compound of claim 5, the tricyclic compound comprising three or more three-ring structures, the tricyclic compound being represented by Formula (IIA) 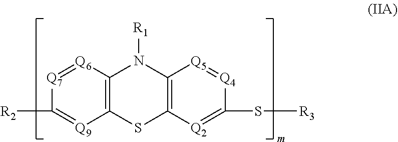
in which
Q2, Q4 to Q7 and Q9 are each independently selected from N or CR5 with the proviso that at most two of Q2 and Q4 to Q5 or two of Q6 to Q7 and Q9 are N,
wherein R1 is selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
R2 and R3 are null or H,
m ranges from 3 to 10,000,
wherein the tricyclic compound has a redox potential of 0.20 V to 2.0 V with reference to Zn/Zn2+ electrode potential under standard conditions.

7. The tricyclic compound of claim 5, the tricyclic compound comprising three or more three-ring structures, the tricyclic compound being represented by Formula (IIB) 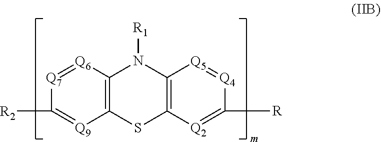
in which
Q2, Q4 to Q7 and Q9 are each independently selected from N or CR5 with the proviso that at most two of Q2 and Q4 to Q5 or two of Q6 to Q7 and Q9 are N,
wherein R1 is selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
R2 and R3 are null or H,
m ranges from 3 to 10,000,
wherein the tricyclic compound has a redox potential of 0.20 V to 2.0 V with reference to Zn/Zn2+ electrode potential under standard conditions.

8. A tricyclic compound of Formula (VII), the tricyclic compound comprising three or more three-ring structure and being represented by Formula (VII) 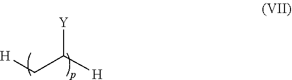
wherein Y is selected from any one of Formula (10a), Formula (10b) and Formula (10c) 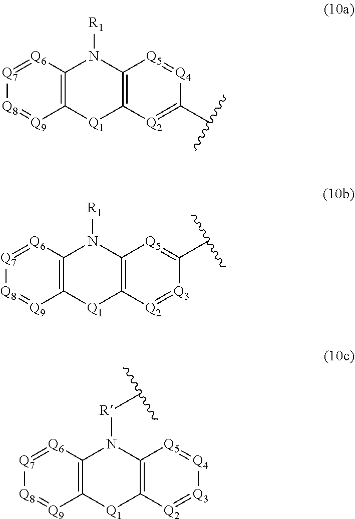
wherein
Q1 is a —O—, —S— or ═NR4,
Q2 to Q5 are each independently selected from N or CR5 with the proviso that at most two of Q2 to Q5 are N and one of Q2 to Q5 is C—X wherein X is Cl, Br, or I,
Q6 to Q9 are each independently selected from N or CR5 with the proviso that at most two of Q6 to Q9 are N,
wherein R′ is selected from a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R1, and R4 are independently selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein p ranges from 3 to 10,000,
wherein the tricyclic compound has a redox potential of 0.20 V to 2.0 V with reference to Zn/Zn²⁺ electrode potential under standard conditions.

9. The tricyclic compound of claim 8, wherein a tricyclic compound is represented by Formula (VIIA), 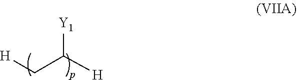
wherein Y1 is selected from any one of Formula (1a), to Formula (3e) 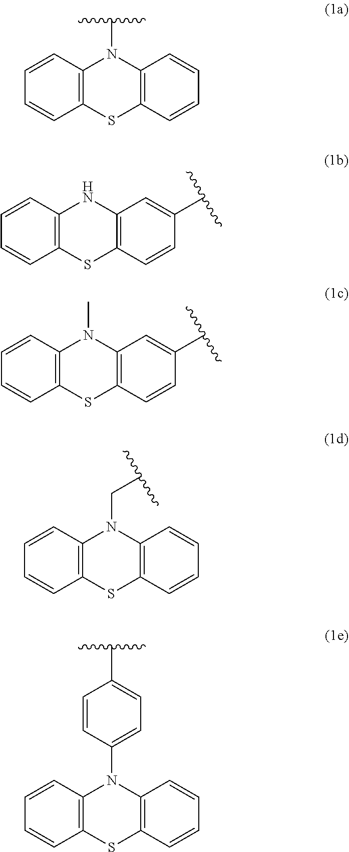 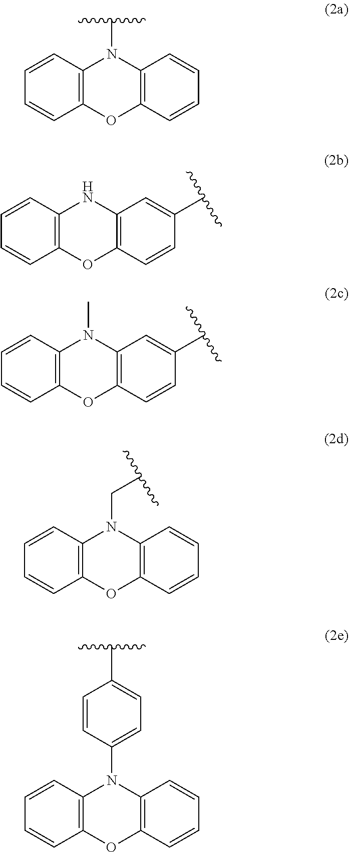 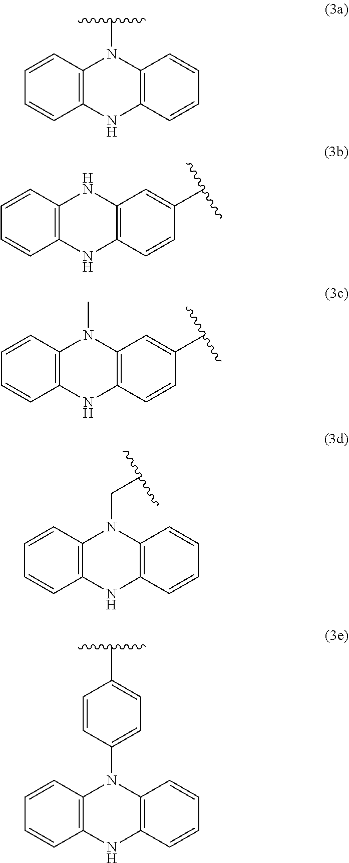
wherein p ranges from 3 to 10,000,
wherein the tricyclic compound as described has a redox potential of 0.20 V to 2.0 V with reference to Zn/Zn²⁺ electrode potential under standard conditions.

10. A method for making the tricyclic compound of claim 4, the tricyclic compound comprising two three-ring structures and being represented by Formula (IV), the method comprising
providing a tricyclic monomer of Formulas (III) 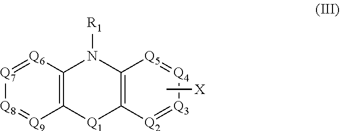
in which
Q1 is a —O—, —S— or ═NR4,
Q2 to Q5 are each independently selected from N or CR5 with the proviso that at most two of Q2 to Q5 are N and one of Q2 to Q5 is C—X wherein X is Cl, Br, or I,
Q6 to Q9 are each independently selected from N or CR5 with the proviso that at most two of Q6 to Q9 are N,
wherein R1, and R4 are independently selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
contacting the tricyclic monomer of Formula (III) with CuI or other coupling reagent capable of performing a carbon-carbon bond formation reaction for a time and under conditions to allow reaction of the tricyclic monomer of Formula (III) with CuI or the other coupling reagent, to provide the dimer of Formula (IV), or
contacting the tricyclic monomer of Formula (III) with a salt for linker L such as Na2S, K2S, Li2S for a time and under conditions to allow reaction of the tricyclic monomer of Formula (III) with the salt for linker L such as Na2S, K2S, Li2S, to provide the dimer of Formula (IV),
wherein L is null when CuI or other coupling reagent is used or O, S, NR1, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S, wherein L links between a ring carbon atom of any one of Q2 to Q5 to a ring carbon atom of any one of Q2 to Q5 of another monomeric moiety,

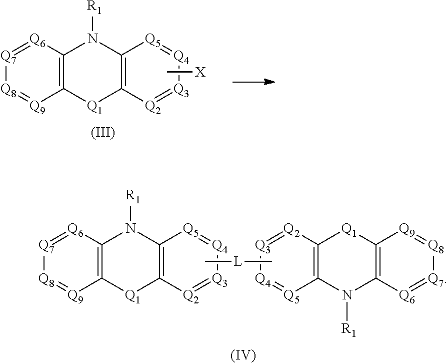

11. The method of claim 10, wherein the tricyclic compound comprising two three-ring structures and represented by Formula (IVA), the method comprising 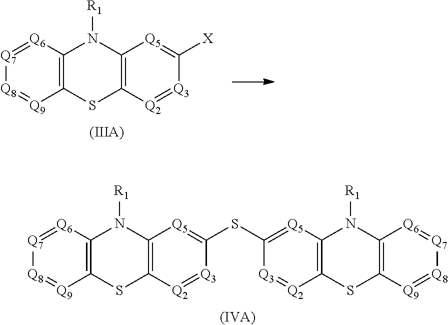
providing a tricyclic monomer of Formula (IIIA)
in which
Q2, Q3 and Q5 are each independently selected from N or CR5 with the proviso that at most two of Q2 to Q5 are N and one of Q2 to Q5 is C—X wherein X is Cl, Br, or I,
Q6 to Q9 are each independently selected from N or CR5 with the proviso that at most two of Q6 to Q9 are N,
wherein R1, and R4 are independently selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S;
contacting the tricyclic monomer of Formula (IIIA) suitable salt of linker —S— for a time and under conditions to allow reaction of the tricyclic monomer of Formula (IIIA) with the salt of linker —S— to provide the dimer of Formula (IVA),
wherein X is a suitable substituent selected from Cl, Br or I.

12. The method of claim 10, wherein tricyclic compound is represented by Formula (IVB), and the method comprises 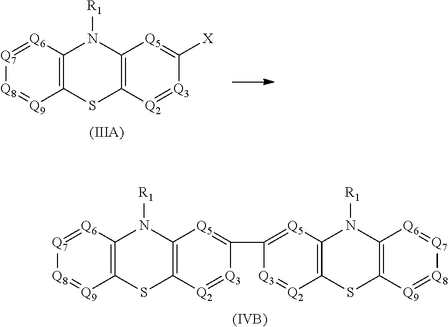
providing a tricyclic monomer of Formula (IIIA)
in which
Q2, Q3 and Q5 are each independently selected from N or CR5 with the proviso that at most two of Q2 to Q5 are N and one of Q2 to Q5 is C—X wherein X is Cl, Br, or I,
Q6 to Q9 are each independently selected from N or CR5 with the proviso that at most two of Q6 to Q9 are N,
wherein R1, and R4 are independently selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S;
contacting the tricyclic monomer of Formula (IIIA) suitable coupling reagent for a time and under conditions to allow C—C bond formation reaction of the tricyclic monomer of Formula (IIIA) with the coupling reagent to provide the dimer of Formula (IVB) herein described,
wherein X is a suitable substituent selected from Cl, Br or I.

13. A method of making the tricyclic compound of claim 5, the tricyclic compound comprising three or more three-ring structures and being represented by Formula (II), the method comprising 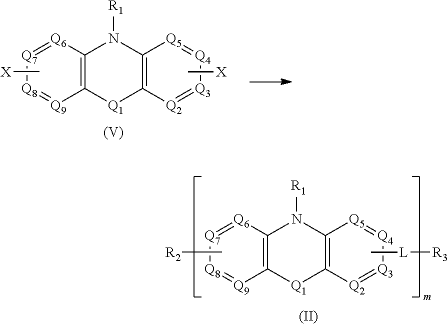
providing a tricyclic monomer of Formula (V)
in which
Q1 is a —O—, —S— or ═NR4,
Q2 to Q9 are each independently selected from N or CR5 with the proviso that at most two of Q2 to Q5 or at most two of Q6 to Q9 are N and one of Q2 to Q5 and one of Q6 to Q9 are C—X wherein X is Cl, Br, or I,
wherein R1, and R4 are independently selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
contacting the tricyclic monomer of Formula (V) with a coupling reagent capable of performing a carbon-carbon bond formation reaction such as CuI for a time and under conditions to allow reaction of the tricyclic monomer of Formula (V) with the coupling reagent to provide the polymer of Formula (II), or
contacting the tricyclic monomer of Formula (V) with a suitable salt of linker L such as any one of Na2S, Li2S, K2S for a time and under conditions to allow reaction of the tricyclic monomer of Formula (V) with a salt for linker L to provide the polymer of Formula (II),
wherein L is null when the coupling reagent is used, or O, S, NR1, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S, wherein L links between a ring carbon atom of any one of Q2 to Q5 to a ring carbon atom of any one of Q6 to Q9 of an adjacent monomeric moiety,
R2 and R3 are null or H,
m ranges from 3 to 10,000.

14. A method of making the tricyclic compound of claim 8, the tricyclic compound comprising three or more three-ring structures of Formula (VII), the method comprising
providing a tricyclic monomer of Formula (VI) 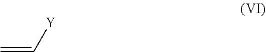
wherein Y is selected from any one of Formula (10a), Formula (10b) and Formula (10c) 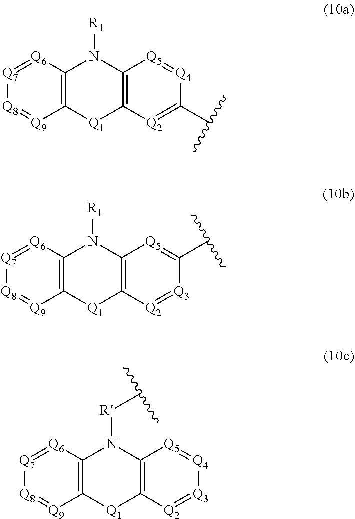
wherein
Q1 is a —O—, —S— or ═NR4,
Q2 to Q5 are each independently selected from N or CR5 with the proviso that at most two of Q2 to Q5 are N and one of Q2 to Q5 is C—X wherein X is Cl, Br, or I,
Q6 to Q9 are each independently selected from N or CR5 with the proviso that at most two of Q6 to Q9 are N,
wherein R′ is selected from a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R1, and R4 are independently selected from H, or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
wherein R5 is selected from H, F, Cl, Br, I, CF3 or a linear or branched, C1-C4 alkyl group, C1-C4 alkenyl group, or an aromatic, heteroaromatic, non-aromatic cycle, or non-aromatic heterocycle containing substituent containing 4-12 carbon atoms and 0-4 heteroatoms, wherein heteroatoms are selected from O, N, and S,
contacting the tricyclic monomer of Formula (VI) with a polymerization initiator or catalyst for a time and under conditions to allow polymerization of the tricyclic monomer of Formula (VI) to provide a polymer of Formula (VII), 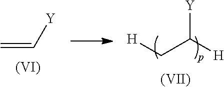
wherein p ranges from 3 to 10,000.

15. An electrode composition comprising
one or more of the tricyclic compounds according to claim 1 together with a binder, and a conductive additive.

16. The electrode composition of claim 15, wherein
the binder is selected from one of Polytetrafluoroethylene (PTFE), Styrene-butadiene or styrene-butadiene rubber (SBR), poly(vinylidene-fluoride) (PVDF), poly(tetrafluoroethylene), sodium carboxymethylcellulose (CMC), styrene-butadiene rubber, polyacrylic acid (PAA), polyvinyl alcohol (PVA), polyethylene glycol (PEG or PEO), polyamide imide (PAI), Polyacrylonitrile (PAN) Xanthan Gum, Gum Arabic, and Agar any combination thereof.

17. The electrode composition of claim 15, wherein
the conductive additive is selected from one of Carbon Black (Acetylene Black, Super P Li, C-Nergy, Ketjen Black-300, Ketjen Black-600), Imerys (Super P, C-Nergy), carbon nanotubes (C-Nano, Tuball), graphene (xGnP Grade R, xGnP Grade H, xGnP Grade C, xGnP Grade M) and Graphite (KS-4, KS-8, KC-4, KC-8), and nickel powder or any combination thereof.

18. The electrode composition of claim 15,
wherein
the binder is present in 1 to 20% by weight of the total electrode composition, and
the conductive additive is present in 5 to 70% by weight of the total electrode composition.

19. A zinc electrochemical cell, comprising a zinc anode, a cathode and an aqueous electrolyte, wherein the cathode comprises one or more of the tricyclic compounds according to claim 1.

20. The zinc electrochemical cell of claim 19, wherein the cathode electrode comprises a tricyclic compound selected from the group consisting of PT (1), CPT (2), MPT (3), PPT (4), PT2S (5), PT2MPT (6), PMPTS (7), PMPT (8), PPTS (9), N-substituted PVPT (10), 2-Substituted PVMT (11), N-Substituted PAPT (12), N-phenyl substituted PSPT (13) or any combination thereof,

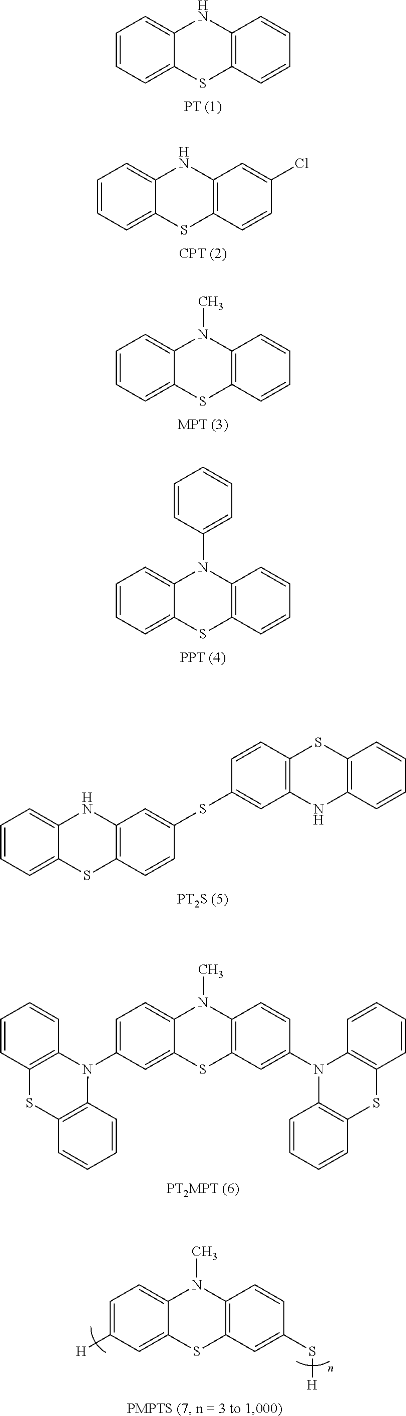

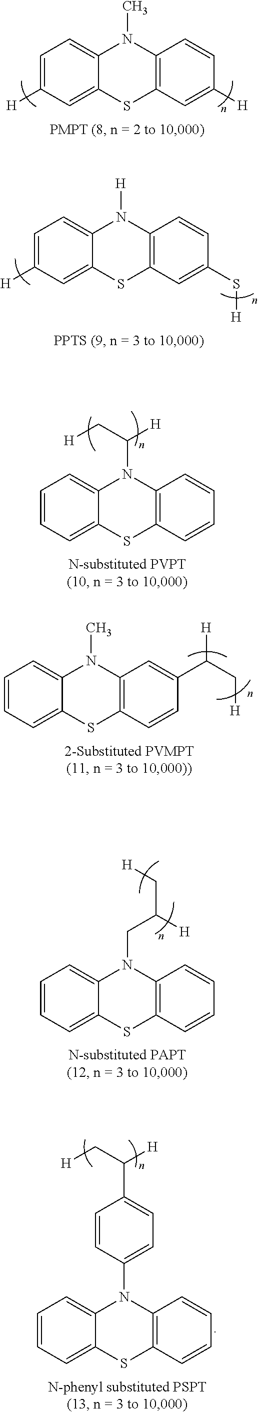

21. The zinc electrochemical cell of claim 19, wherein
the cathode further comprises a binder, and a conductive additive.

22. The zinc electrochemical cell of claim 19, wherein
the aqueous electrolyte has a pH value from 2 to 10 at room temperature,
the aqueous electrolyte comprises at least one salt, wherein the at least one salt comprises a cation selected from Li⁺, Na⁺, K⁺, NH₄⁺, Mg²⁺, Zn²⁺ or any combination thereof, and an anion counterion selected from F⁻, Cl⁻, Br⁻, I⁻, SO₄²⁻, ClO₄⁻, OAc⁻, TFSI⁻, OTf⁻, TFA⁻ and HCO₂⁻ or any combination thereof.

23. The zinc electrochemical cell of claim 22, wherein the at least one salt is selected from Zn₂SO₄, Zn(OCl₄)₂, Zn(NO₃)₂, ZnF₂, ZnCl₂, ZnBr₂, ZnI₂, Zn(OAc)₂, Zn(OTf)₂, Zn(TFSI)₂, Zn(BF₄)₂, optionally in combination with LiF, LiCl, LiBr, LiI, LiClO₄, LiTFSI, LiOTf, LiTFA, LiOAc, Li₂SO₄, LiNO₃, Li-formate, NaF, NaCl, NaBr, NaI, Na₂SO₄, NaClO₄, NaOTf, NaOAc, NaTFA, KF, KCl, KBr, KI, K₂SO₄, KClO₄, KOTf, KTFSI, KOAc, KTFA, NH₄Cl, MgSO₄, wherein concentration of each salt is present at a concentration equal to or greater than 0.01 M and the total concentration in the electrolyte is equal to or less than 30 M.

24. A battery comprising one or more electrochemical cells of claim 19.
